(19) United States
(12) Patent Application Publication
(10) Pub. No.: US 2008/0248037 A1
(43) Pub. Date: Oct. 9, 2008

(54) Dr5 Antibodies and Uses Thereof

(75) Inventors: Bing Li, Foster City, CA (US); Sachdev S. Sidhu, San Francisco, CA (US)

Correspondence Address: GENENTECH, INC., 1 DNA WAY, SOUTH SAN FRANCISCO, CA, 94080, US

(73) Assignee: Genentech, Inc., San Francisco, CA (US)

(21) Appl. No.: 11/547,457

(22) PCT Filed: Apr. 4, 2005
(86) PCT No.: PCT/US05/11257

§ 371 Date: Mar. 10, 2008

Related U.S. Application Data
(60) Provisional application No. 60/559,928, filed on Apr. 6, 2004.

(51) Int. Cl. A61K 39/395; C07K 16/18; A61P 43/00

(52) U.S. Cl. 424/138.1; 530/387.1; 530/387.3; 424/172.1

(57) ABSTRACT

The application provides antibodies which specifically bind to DR5 receptor. The anti-DR5 antibodies optionally contain CDR sequences identified using phage-display techniques. The DR5 antibodies can be used, for example, in methods where a modulation of the biological activities of Apo-2L and/or Apo-2L receptors is desired, including cancer and immune-related conditions.

### RELATED APPLICATIONS

This application claims priority under Section 119 to provisional application No. 60/559,928 filed Apr. 6, 2004, the contents of which are hereby incorporated by reference.

### FIELD OF THE INVENTION

The present invention relates to antibodies which bind to DR5 receptors. Such antibodies can be used, for example, in methods where a modulation of the biological activities of Apo-2L and/or Apo-2L receptors is desired.

### BACKGROUND OF THE INVENTION

Various molecules, such as tumor necrosis factor-alpha (“TNF-alpha”), tumor necrosis factor-beta (“TNF-beta” or “lymphotoxin-alpha”), lymphotoxin-beta (“LT-beta”), CD30 ligand, CD27 ligand, CD40 ligand, OX-40 ligand, 4-1BB ligand, Apo-1 ligand (also referred to as Fas ligand or CD95 ligand), Apo-2 ligand (also referred to as Apo2L or TRAIL), Apo-3 ligand (also referred to as TWEAK), APRIL, OPG ligand (also referred to as RANK ligand, ODF, or TRANCE), and TALL-1 (also referred to as BlyS, BAFF or THANK) have been identified as members of the tumor necrosis factor (“TNF” ) family of cytokines (See, e.g., Gruss and Dower, Blood, 85:3378-3404 (1995); Schmid et al., Proc. Natl. Acad. Sci., 83:1881 (1986); Dealtry et al., Eur. J. Immunol., 17:689 (1987); Pitti et al., J. Biol. Chem., 271:12687-12690 (1996); Wiley et al., Immunity, 3:673-682 (1995); Browning et al., Cell, 72:847-856 (1993); Armitage et al. Nature, 357:80-82 (1992), WO 97/01633 published Jan. 16, 1997; WO 97/25428 published Jul. 17, 1997; Marsters et al., Curr Biol., 8:525-528 (1998); Chicheportiche et al., Biol. Chem., 272:32401-32410 (1997); Hahne et al., J. Exp. Med., 188:1185-1190 (1998); WO98/28426 published Jul. 2, 1998; WO98/46751 published Oct. 22, 1998; WO/98/18921 published May 7, 1998; Moore et al., Science, 285:260-263 (1999); Shu et al., J. Leukocyte Biol., 65:680 (1999); Schneider et al., J. Exp. Med., 189:1747-1756 (1999); Mukhopadhyay et al., J. Biol. Chem., 274:15978-15981 (1999)). Among these molecules, TNF-alpha, TNF-beta, CD30 ligand, 4-1BB ligand, Apo-1 ligand, Apo-2 ligand (Apo2L/TRAIL) and Apo-3 ligand (TWEAK) have been reported to be involved in apoptotic cell death.

Apo2L/TRAIL was identified several years ago as a member of the TNF family of cytokines. (see, e.g., Wiley et al., Immunity, 3:673-682 (1995); Pitti et al., J. Biol. Chem., 271:12697-12690 (1996)) The full-length human Apo2L/TRAIL polypeptide is a 281 amino acid long, Type II transmembrane protein. Some cells can produce a natural soluble form of the polypeptide, through enzymatic cleavage of the polypeptide's extracellular region (Mariani et al., J. Cell. Biol., 137:221-229 (1997)). Crystallographic studies of soluble forms of Apo2L/TRAIL reveal a homotrimeric structure similar to the structures of TNF and other related proteins (Hymowitz et al., Molec. Cell, 4:563-571 (1999); Hymowitz et al., Biochemistry, 39:633-644 (2000)). Apo2L/TRAIL, unlike other TNF family members however, was found to have a unique structural feature in that three cysteine residues (at position 230 of each subunit in the homotrimer) together coordinate a zinc atom, and that the zinc binding is important for trimer stability and biological activety. (Hymowitz et al., supra; Bodmer et al., J. Biol. Chem., 275:20632-20637 (2000))

It has been reported in the literature that Apo2L/TRAIL may play a role in immune system modulation, including autoimmune diseases such as rheumatoid arthritis, and in the treatment of HIV (see, e.g., Thomas et al., J. Immunol., 161:2195-2200 (1998); Johnsen et al., Cytokine, 11:664-672 (1999); Griffith et al., J. Exp. Med., 189:1343-1353 (1999); Song et al., J. Exp. Med., 191:1095-1103 (2000); Jeremias et al., Eur. J. Immunol., 28:143-152 (1998); Katsikis et al., J. Exp. Med., 186:1365-1372 (1997); Miura et al., J. Exp. Med., 193:651-660 (2001)).

Soluble forms of Apo2L/TRAIL have also been reported to induce apoptosis in a variety of cancer cells in vitro, including colon, lung, breast, prostate, bladder, kidney, ovarian and brain tumors, as well as melanoma, leukemia, and multiple myelcoma (see, e.g., Wiley et al., supra; Pitti et al., supra; Rieger et al., FEBS Letters, 427:124-128 (1998); Ashkenazi et al., J. Clin. Invest., 104:155-162 (1999); Walczak et al., Nature Med., 5:157-163 (1999); Keane et al., Cancer Research, 59:734-741 (1999); Mizutani et al., Clin. Cancer Res., 5:2605-2612 (1999); Gazitt, Leukemia, 13:1817-1824 (1999); Yu et al., Cancer Res., 60:2384-2389 (2000); Chinnaiyan et al., Proc. Natl. Acad. Sci., 97:1754-1759 (2000)). In vivo studies in murine tumor models further suggest that Apo2L/TRAIL, alone or in combination with chemotherapy or radiation therapy, can exert substantial anti-tumor effects (see, e.g., Ashkenazi et al., supra; Walzcak et al., supra; Gliniak et al., Cancer Res., 59:6153-6158 (1999); Chinnaiyan et al., supra; Roth et al., Biochem. Biophys. Res. Comm., 265:1999 (1999)). In contrast to many types of cancer cells, most normal human cell types appear to be resistant to apoptosis induction by certain recombinant forms of Apo2L/TRAIL (Ashkenazi et al., supra; Walzcak et al., supra). Jo et al. has reported that a polyhistidine-tragged soluble form of Apo2L/TRAIL induced apoptosis in vitro in normal isolated human, but not non-human, hepatocytes (Jo et al., Nature Med., 6:564-567 (2000); see also, Nagata, Nature Med., 6:502-503 (2000)). It is believed that certain recombinant Apo2L/TRAIL preparations may vary in terms of biochemical properties and biological activities on diseased versus normal cells, depending, for example, on the presence or absence of a tag molecule, zinc content, and % trimer content (See, Lawrence et al., Nature Med., Letter to the Editor, 7:383-385 (2001); Qin et al., Nature Med., Letter to the Editor, 7:385-386 (2001)).

Induction of various cellular responses mediated by such TNF family cytokines is believed to be initiated by their binding to specific cell receptors. Previously, two distinct TNF receptors of approximately 55-kDa (TNFR1) and 75-kDa (TNFR2) were identified (Hohman et al., J. Biol. Chem., 264:14927-14934 (1989); Brockhaus et al., Proc. Natl. Acad. Sci., 87:3127-3131 (1990); EP 417,563, published Mar. 20, 1991; Loetscher et al., Cell, 61:351 (1990); Schall et al., Cell, 61:361 (1990); Smith et al., Science, 248:1019-1023 (1990); Lewis et al., Proc. Natl. Acad. Sci., 88:2830-2834 (1991); Goodwin et al., Mol. Cell. Biol., 11:3020-3026 (1991)). Those TNFRs were found to share the typical structure of cell surface receptors including extracellular, transmembrane arid intracellular regions. The extracellular portions of both receptors were found naturally also as soluble TNF-binding proteins (Nophar, Y. et al., EMBO J., 9:3269 (1990); and Kohno, T. et al., Proc. Natl. Acad. Sci. U.S.A., 87:8331 (1990); Hale et al., J. Cell. Biochem. Supplement 15F, 1991, p. 113 (P424)).

The extracellular portion of type 1 and type 2 TNFRs (TNFR1 and TNFR2) contains a repetitive amino acid sequence pattern of four cysteine-rich domains (CRDs) designated 1 through 4, starting from the NH₂-terminus. (Schall et al., supra; Loetscher et al., supra; Smith et al., supra; Nophar et al., supra; Kohno et al., supra; Banner et al., Cell, 73:431-435 (1993)). A similar repetitive pattern of CRDs exists in several other cell-surface proteins, including the p75 nerve growth factor receptor (NGFR) (Johnson et al., Cell, 47:545 (1986); Radeke et al., Nature, 325:593 (1987)), the B cell antigen CD40 (Stamemikovic et al., EMBO J., 8:1403 (1989)), the T cell antigen OX40 (Mallet et al., EMBO J., 9:1063 (1990)) and the Fas antigen (Yonehara et al., supra and Itoh et al., Cell, 66:233-243 (1991)). CRDs are also found in the soluble TNFR (sTNFR)-like T2 proteins of the Shope and myxoma poxviruses (Upton et al., Virology, 160:20-29 (1987); Smith et al., Biochem. Biophys. Res. Commun., 176:335 (1991); Upton et al., Virology, 184:370 (1991)). Optimal alignment of these sequences indicates that the positions of the cysteine residues are well conserved. These receptors are sometimes collectively referred to as members of the TNF/NGF receptor superfamily.

The TNF family ligands identified to date, with the exception of lymphotoxin-beta, are typically type II transmembrane proteins, whose C-terminus is extracellular. In contrast, cost receptors in the TNF receptor (TNFR) family identified to date are typically type I transmembrane proteins. In both the TNF ligand and receptor families, however, homology identified between family members has been found mainly in the extracellular domain (“ECD”). Several of the TNF family cytokines, including TNF-alpha, Apo-1 ligand an d CD40 ligand, are cleaved proteolytically at the cell surface; the resulting protein in each case typically forms a homotrimeric molecule that functions as a soluble cytokine. TNF receptor family proteins are also usually cleaved proteolytically to release soluble receptor ECDs that can function as inhibitors of the cognate cytokines.

Pan et al. have disclosed another TNF receptor family member referred to as “DR4” (Pan et al., Science, 276:111-113 (1997); see also WO98/32856 published Jul. 30, 1998; WO99/37684 published Jul. 29, 1999; WO 00/73349 published Dec. 7, 2000; U.S. Pat. No. 6,433,147 issued Aug. 13, 2002; U.S. Pat. No. 6,461,823 issued Oct. 8, 2002, and U.S. Pat. No. 6,342,383 issued Jan. 29, 2002). DR4 is reported to contain a cytoplasmic death domain capable of engaging the cell suicide apparatus. Pan et al. disclose that DR4 is believed to be a receptor for the ligand known as Apo2L/ TRAIL.

In Sheridan et al., Science, 277:818-821 (1997) and Pan et al., Science, 277:815-818 (1997), another molecule believed to be a receptor for Apo2L/TRAIL is described (see also, WO98/51793 published Nov. 19, 1998; WO98/41629 published Sep. 24, 1998). That molecule is referred to as DR5 (it has also been alternatively referred to as Apo-2; TRAIL-R, TR6, Tango-63, hAPO8, TRICK2 or KILLER (see, e.g., Screaton et al., Curr. Biol., 7:693-696 (1997); Walczak et al., EMBO J., 16:5386-5387 (1997); Wu et al., Nature Genetics, 17:141-143 (1997); WO98/35986 published Aug. 20, 1998; EP870,827 published Oct. 14, 1998; WO98/46643 published Oct. 22, 1998; WO99/02653 published Jan. 21, 1999; WO99/09165 published Feb. 25, 1999; WO99/11791 published Mar. 11, 1999; US 2002/0072091 published Aug. 13, 2002; US 2002/0098550 published Dec. 7, 2001; U.S. Pat. No. 6,313,269 issued Dec. 6, 2001; US 2001/0010924 published Aug. 2, 2001; US 2003/01255540 published Jul. 3, 2003; US 2002/0160446 published Oct. 31, 2002, US 2002/0048785 published Apr. 25, 2002; U.S. Pat. No. 6,569,642 issued May 27, 2003; U.S. Pat. No. 6,072,047 issued Jun. 6, 2000; U.S. Pat. No. 6,642,358 issued Nov. 4, 2003). Like DR4, DR5 is reported to contain a cytoplasmic death domain and be capable of signaling apoptosis. The crystal structure of the complex formed between Apo-2L/TRAIL and DR5 is described in Hymowitz et al., Molecular Cell, 4:563-571 (1999).

A further group of recently identified receptors are referred to as “decoy receptors,” which are believed to function as inhibitors, rather than transducers of signaling. This group includes DCR1 (also referred to as TRID, LIT or TRAIL-R3) (Pan et al., Science, 276:111-113 (1997); Sheridan et al., Science, 277:818-821 (1997); McFarlane et al., J. Biol. Chem., 272:25417-25420 (1997); Schneider et al., FEBS Letters, 416:329-334 (1997); Degli-Esposti et al., J. Exp. Med., 186:1165-1170 (1997); and Mongkolsapaya et al., J. Immunol., 160:3-6 (1998)) and DCR2 (also called TRUNDD or TRAIL-R4) (Marsters et al., Curr. Biol., 7:1003-1006 (1997); Pan et al., FEBS Letters, 424:41-45 (1998); Degli-Esposti et al., Immunity, 7:813-820 (1997)), both cell surface molecules, as well as OPG (Simonet et al., supra; Emery et al., infra) and DCR3 (Pitti et al., Nature, 396:699-703 (1998)), both of which are secreted, soluble proteins. Apo2L/TPAIL has been reported to bind those receptors referred to as DcR1, DcR2 and OPG.

Apo2L/TPAIL is believed to act through the cell surface “death receptors” DR4 and DR5 to activate caspases, or enzymes that carry out the cell death program. Upon ligand binding, both DR4 and DR5 can trigger apoptosis independently by recruiting and activating the apoptosis initiator, caspase-8, through the death-domain-containing adaptor molecule referred to as FADD/Mort1 (Kischkel et al., Immunity, 12:611-620 (2000); Sprick et al., Immunity, 12:599-609 (2000); Bodmer et al., Nature Cell Biol., 2:241-243 (2000)). In contrast to DR4 and DR5, the DcR1 and DcR2 receptors do not signal apoptosis.

For a review of the TNF family of cytokines and their receptors, see Ashkenazi and Dixit, Science, 281:1305-1308 (1998); Ashkenazi and Dixit, Curr. Opin. Cell Biol., 11:255-260 (2000); Golstein, Curr. Biol., 7:750-753 (1997); Gruss and Dower, supra; Nagata, Cell, 88:355-365 (1997); Locksley et al., Cell, 104:487-501 (2001); Wallach, “TNF Ligand and TNF/NGF Receptor Families”, Cytokine Reference, Academic Press, 2000, pages 377-411.

### SUMMARY OF THE INVENTION

The present invention provides antibodies that bind DR5 receptors. Optionally, the antibody is in monomer, dimer, trimer, tetramer, or higher oligomeric forms. Optionally the antibody is a chimeric molecule or fusion protein comprising the antibody fused to a heterologous peptide sequence facilitating the formation of oligomeric complexes. In one embodiment, the antibody inhibits the interaction of Apo-2L with DR5 receptor. Optionally, the antibody is an agonist of at least one Apo-2L associated biological activity, for example, the induction of apoptosis via the DR5 receptor.

In certain embodiments, the anti-DR5 antibodies comprise one or more amino acid residues or sequences provided as CDR-H1, CDR-H2, or CDR-H3 in FIG. 6, 7 or 8. Optionally, the anti-DR5 antibodies comprise one or more amino acid sequences having at least 80% identity to those sequences referred to as CDR-H1, CDR-H2 or CDR-H3 in FIG. 6, 7, or 8. In further embodiments, the anti-DR5 antibodies may comprise one or more amino acid sequences having at least 90% or at least 95% identity to those sequences referred to as CDR-H1, CDR-H2 or CDR-H3 in FIG. 6, 7, or 8. Optionally, the DR5 antibody of the invention binds to a DR5 receptor at a concentration range of about 0.1 nM to about 20 mM as measured in a BIAcore binding assay (such as disclosed in the Examples below). Optionally, the DR5 antibodies of the invention exhibit an Ic 50 value of about 0.6 nM to about 18 mM as measured in a BIAcore binding assay (such as disclosed in the Examples below).

Related embodiments of the invention include a nucleic acid molecule encoding an antibody comprising one or more such amino acid sequences. Further embodiments of the invention include vectors comprising a nucleic acid molecule encoding such an antibody as well as host cells comprising these vectors (e.g. E. coli). Additional embodiments of the invention include methods of making DR5 receptor antibody, comprising the steps of: providing a host cell with a vector that includes a nucleic acid sequence encoding an antibody of the invention; (b) providing culture media; (c) culturing the host cell in the culture media under conditions sufficient to express the antibody; (d) recovering the antibody from the host cell or culture media; and (e) purifying the antibody.

The DR5 receptor antibodies of the invention may be modified using one of the wide variety of methods known in the art. In preferred embodiments of the invention, an antibody of the invention is linked to a heterologous molecule or polypeptide sequence. Optionally, the heterologous polypeptide sequence is a leucine zipper domain. Optionally the heterologous sequence comprises the amino acid sequence glycine-glycine-methionine. Optionally, the antibody may be conjugated or linked to one or more linker molecules or polyol groups.

In certain embodiments of the invention, the antibody blocks or inhibits the interaction between Apo-2L and DR5. Optionally, the antibody induces apoptosis in one or more mammalian cells.

Also provided herein is a composition comprising at least one of the antibodies described above in a carrier. Preferably, this composition is sterile. In addition, the invention provides methods or preparing the compositions described above. In particularly desirable embodiments, the resulting compositions are pharmaceutically acceptable formulations.

Isolated nucleic acids encoding the antibodies described herein, are also provided, and may be used, e.g., for in vivo or ex vivo gene therapy.

Other embodiments of the invention are methods of modulating the biological activity of Apo-2L and/or an Apo-2L receptor in mammalian cells. A preferred embodiment of the invention is a method of inducing apoptosis in mammalian cells, comprising exposing mammalian cells to an effective amount of a DR5 receptor antibody described herein. The mammalian cells may be, e.g., cancer cells. In still further aspects, the invention provides methods for treating a disorder, such as canner or an immune related disorder, in a mammal comprising administering to the mammal, optionally by injection or infusion, an effective amount of a DR5 receptor antibody provided by the present invention. Optionally, the disorder is cancer, and more particularly, is a breast, lung, colon (or colorectal), or glioma cancer. The antibodies described herein can be administered alone or together with another agent.

In additional embodiments, the invention provides kits comprising a container comprising an antibody described herein and instructions for using the antibody; such as for using the antibody to treat a disorder against which the antibody is effective. Optionally, the disorder is cancer, and more particularly, is a breast, lung, colon (or colorectal) or glioma cancer.

Yet another embodiment of the invention is an article of manufacture comprising a container which includes an antibody described herein, and printed instructions for use of the antibody. Optionally, the container is a bottle, vial, syringe, or test tube. Optionally, the article of manufacture comprises a second container which includes water-for-injection, saline, Ringer's solution, or dextrose solution.

In particular, there are provided the following embodiments set forth in claim format:
1. An isolated anti-DR5 antibody comprising one or more amino acid sequences set forth in FIG. 6, 7 or 8.
2. An isolated anti-DR5 antibody, comprising a heavy chain and a light chain, wherein the heavy chain comprises a variable region comprising one or more amino acid sequences set forth in FIG. 6, 7 or 8.
3. The antibody of claim 2, wherein the heavy chain and the light chain are connected by a flexible linker to form a single-chain antibody.
4. The antibody of claim 3, which is a single-chain Fv antibody.
5. The antibody of claim 2, which is a Fab antibody.
6. The antibody of claim 2, which is fully human.
7. The antibody of claim 1 or claim 2 which specifically binds DR5 receptor and does not bind DR4 receptor, DcR1 receptor or DcR2 receptor.
8. The antibody of claim 1 or claim 2 which induces apoptosis in at least one type of mammalian cancer cells.
9. The antibody of claim 1 or claim 2 which blocks or inhibits binding of Apo-2 ligand to DR5 receptor.
10. A composition comprising an antibody of any of claims 1 to 9 and a carrier.
11. A method of treating a disorder in a mammal, comprising administering the composition of claim 10.
12. The method of claim 11, wherein the disorder is an immune-related disorder.
13. The method of claim 11, wherein the disorder is cancer.

### BRIEF DESCRIPTION OF THE DRAWINGS

FIG. 1 shows the nucleotide sequence of human Apo-2 ligand cDNA (SEQ ID NO:2) and its derived amino acid sequence (SEQ ID NO:1). The “N” at nucleotide position 447 is used to indicate the nucleotide base may be a “T” or “G”.

FIGS. 2A and 2B show the nucleotide sequence of a cDNA (SEQ ID NO:4) for full length human DR4 and its derived amino acid sequence (SEQ ID NO: 3). The respective nucleotide and amino acid sequences for human DR4 are also reported in Pan et al., Science, 276:111 (1997).

FIG. 3A shows the 411 amino acid sequence (SEQ ID NO:5) of human DR5 as published in WO 98/51793 on Nov. 19, 1998. A transcriptional splice variant of human DR5 is known in the art. This DR5 splice variant encodes the 440 amino acid sequence (SEQ ID NO:6) of human DR5 shown in FIGS. 3B and 3C as published in WO 98/35986 on Aug. 20, 1998.

FIGS. 4A-F shows the polynucleotide sequence (SEQ ID NO:7) encoding the vector pS2072 referred to in Example 1. The coding sequences of the respective CDRs of the light chain and heavy chain are underlined.

FIGS. 5A-G shows the polynucleotide sequence (SEQ ID NO:8) encoding the vector pV-0350-2 referred to in Example 3. The coding sequences of the respective CDRs of the light chain and heavy chain are underlined.

FIGS. 6A-C shows the I.D. (Identifier) assigned to each clone selected from the scFv library (Example 1) and the respective amino acid sequences (SEQ ID NOs: 9-77) for the CDRs of the heavy chain (CDR-H1; CDR-H2, CDR-H3).

FIG. 7 shows the I.D. (Identifier) assigned to each clone selected from the scFv library (Example 2) and the respective amino acid sequences (SEQ ID NOs: 78-110) for the CDRs of the heavy chain (CDR-H1; CDR-H2, CDR-H3).

FIG. 8 shows the I.D. (Identifier) assigned to each clone selected from the Fab library (Example 3) and the respective amino acid sequences (SEQ ID NOs:111-115) for the CDRs of the heavy chain (CDR-H1; CDR-H2, CDR-H3).

FIG. 9 is a graph illustrating the results of an ELISA testing binding of Fab antibody BdF2 to the human DR5-ECD polypeptide.

FIG. 10 shows the results of an AlamarBlue bioassay testing ability of Apo2L and Fab antibody BdF2 to induce apoptosis in Colo205 tumor cells in vitro.

### DETAILED DESCRIPTION OF THE INVENTION

### A. Definitions

“TNF family member” is used in a broad sense to refer to various polypeptides that share some similarity to tumor necrosis factor (TNF) with respect to structure or function. Certain structural and functional characteristics associated with the TNF family of polypeptides are known in the art and described, for example, in the above Background of the Invention. Such polypeptides include but are not limited to those polypeptides referred to in the art as TNF-alpha, TNF-beta, CD40 ligand, CD30 ligand, CD27 ligand, OX-40 ligand, 4-1BB ligand, Apo-1 ligand (also referred to as Fas ligand or CD95 ligand), Apo-2L/TRAIL (also referred to as TRAIL), Apo-3 ligand (also referred to as TWEAK), APRIL, OPG ligand (also referred to as RANK ligand, ODF, or TRANCE), and TALL-1 (also referred to as BlyS, BAFF or THANK) (See, e.g., Gruss and Dower, Blood 1995, 85:3378-3404; Pitti et al., J. Biol. Chem. 1996, 271:12687-12690; Wiley et al., Immunity 1995, 3:673-682; Browning et al., Cell 1993, 72:847-856; Armitage et al. Nature 1992, 357:80-82, PCT Publication Nos. WO 97/01633; and WO 97/25428; Marsters et al., Curr. Biol. 1998, 8:525-528; Chicheportiche et al., Biol. Chem. 1997, 272:32401-32410; Hahne et al., J. Exp. Med. 1998, 188:1185-1190; PCT Publication Nos. WO98/28426; WO98/46751; and WO/98/18921; Moore et al., Science 1999, 285:260-263; Shu et al., J. Leukocyte Biol. 1999, 65:680; Schneider et al., J. Exp. Med. 1999, 189:1747-1756; Mukhopadhyay et al., J. Biol. Chem. 1999, 274:15978-15981).

“DR5 receptor antibody”, “DR5 antibody”, or “anti-DR5 antibody” is used in a broad sense to refer to antibodies that bind to at least one form of a DR5 receptor. Optionally the DR5 antibody is fused or linked to a heterologous sequence or molecule. Preferably the heterologous sequence allows or assists the antibody to form higher order or oligomeric complexes. Optionally, the DR5 antibody binds to DR5 receptor but does not bind or cross-react with any additional Apo-2L receptor (e.g. DR4, DcR1, or DcR2). Optionally the antibody is an agonist of DR5 signalling activity.

Optionally, the DR5 antibody of the invention binds to a DR5 receptor at a concentration range of about 0.1 nM to about 20 mM as measured in a BIAcore binding assay (such as, for example, disclosed in the Examples below). Optionally, the DR5 antibodies of the invention exhibit an Ic 50 value of about 0.6 nM to about 18 mM as measured in a BIAcore binding assay (such as, for example, disclosed in the Examples below).

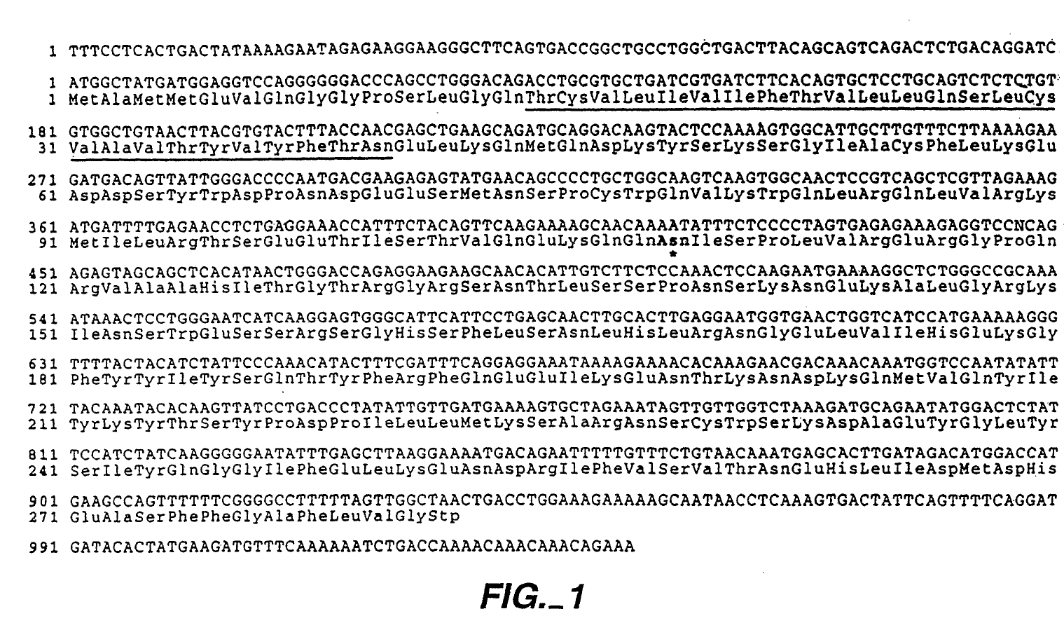

The terms “Apo2L/TRAIL”, “Apo-2L”, and “TRAIL” are used herein to refer to a polypeptide sequence which includes amino acid residues 114-281, inclusive, 95-281, inclusive, residues 92-281, inclusive, residues 91-281, inclusive, residues 41-281, inclusive, residues 15-281, inclusive, or residues 1-281, inclusive, of the amino acid sequence shown in FIG. 1, as well as biologically active fragments, deletional, insertional, or substitutional variants of the above sequences. In one embodiment, the polypeptide sequence comprises residues 114-281 of FIG. 1, and optionally, consists of residues 114-281 of FIG. 1. Optionally, the polypeptide sequence comprises residues 92-281 or residues 91-281 of FIG. 1. The Apo-2L polypeptides may be encoded by the native nucleotide sequence shown in FIG. 1. Optionally, the codon which encodes residue Pro119 of FIG. 1 may be “CCT” or “CCG”. In other embodiments, the fragments or variants are biologically active and have at least about 80% amino acid sequence identity, more preferably at least about 90% sequence identity, and even more preferably, at least 95%, 96%, 97%, 98%, or 99% sequence identity with any one of the above recited Apo2L/TRAIL sequences. Optionally, the Apo2L/TRAIL polypeptide is encoded by a nucleotide sequence which hybridizes under stringent conditions with the encoding polynucleotide sequence provided in FIG. 1. The definition encompasses substitutional variants of Apo2L/TRAIL in which at least one of its native amino acids are substituted by an alanine residue. Particular substitutional variants of the Apo2L/TRAIL include those in which at least one amino acid is substituted by an alanine residue. These substitutional variants include those identified, for example, as “D203A”; “D218A” and “D269A.” This nomenclature is used to identify Apo2L/TRAIL variants wherein the aspartic acid residues at positions 203, 218, and/or 269 (using the numbering shown in FIG. 1) are substituted by alanine residues. Optionally, the Apo2L variants may comprise one or more of the alanine substitutions which are recited in Table I of published PCT application WO 01/00832. Substitutional variants include one or more of the residue substitutions identified in Table I of WO 01/00832 published Jan. 4, 2001. The definition also encompasses a native sequence Apo2L/TRAIL isolated from an Apo2L/TRAIL source or prepared by recombinant or synthetic methods. The Apo2L/TRAIL of the invention includes the polypeptides referred to as Apo2L/TRAIL or TRAIL disclosed in PCT Publication Nos. WO97/01633 and WO97/25428. The terms “Apo2L/TRAIL” or “Apo2L” are used to refer generally to forms of the Apo2L/TRAIL which include monomer, dimer or trimer forms of the polypeptide. All numbering of amino acid residues referred to in the Apo2L sequence use the numbering according to FIG. 1, unless specifically stated otherwise. For instance, “D203” or “Asp203” refers to the aspartic acid residue at position 203 in the sequence provided in FIG. 1.

The term “Apo2L/TRAIL extracellular domain” or “Apo2L/TRAIL ECD” refers to a form of Apo2L/TRAIL which is essentially free of transmembrane and cytoplasmic domains. Ordinarily, the ECD will have less than 1% of such transmembrane and cytoplasmic domains, and preferably, will have less than 0.5% of such domains. It will be understood that any transmembrane domain(s) identified for the polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified. In preferred embodiments, the ECD will consist of a soluble, extracellular domain sequence of the polypeptide which is free of the transmembrane and cytoplasmic or intracellular domains (and is not membrane bound). Particular extracellular domain sequences of Apo-2L/TRAIL are described in PCT Publication Nos. WO97/01633 and WO97/25428.

The term “Apo2L/TRAIL monomer” or “Apo2L monomer” refers to a covalent chain of an extracellular domain sequence of Apo2L.

The term “Apo2L/TRAIL dimer” or “Apo2L dimer” refers to two Apo-2L monomers joined in a covalent linkage via a disulfide bond. The term as used herein includes free standing Apo2L dimers and Apo2L dimers that are within trimeric forms of Apo2L (i.e., associated with another, third Apo2L monomer).

The term “Apo2L/TRAIL trimer” or “Apo2L trimer” refers to three Apo2L monomers that are non-covalently associated.

The term “Apo2L/TRAIL aggregate” is used to refer to self-associated higher oligomeric forms of Apo2L/TRAIL, such as Apo2L/TRAIL trimers, which form, for instance, hexameric and nanomeric forms of Apo2L/TRAIL. Determination of the presence and quantity of Apo2L/TRAIL monomer, dimer, or trimer (or other aggregates) may be made using methods and assays known in the art (and using commercially available materials), such as native size exclusion HPLC (“SEC”), denaturing size exclusion using sodium dodecyl sulphate (“SDS-SEC”), reverse phase HPLC and capillary electrophoresis.

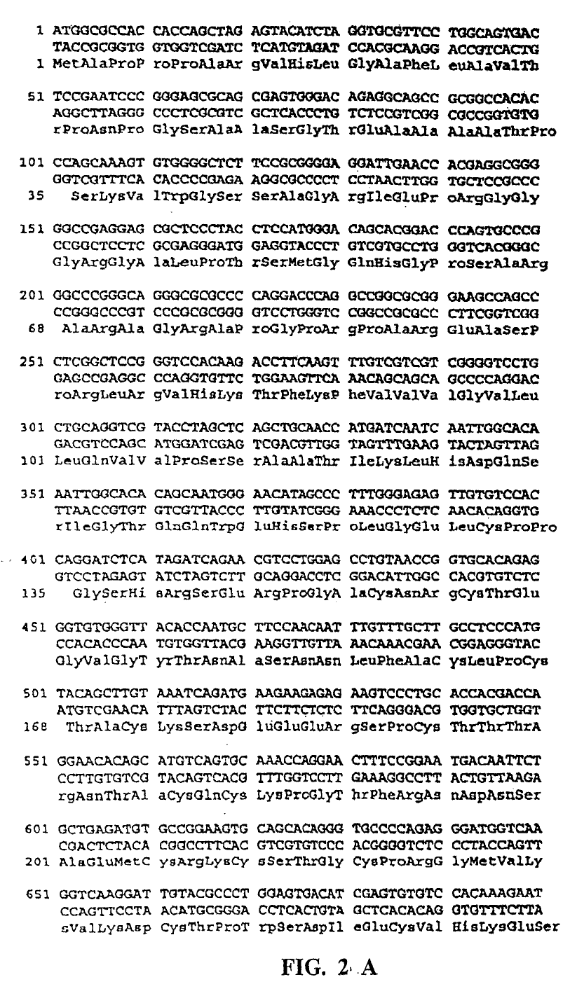

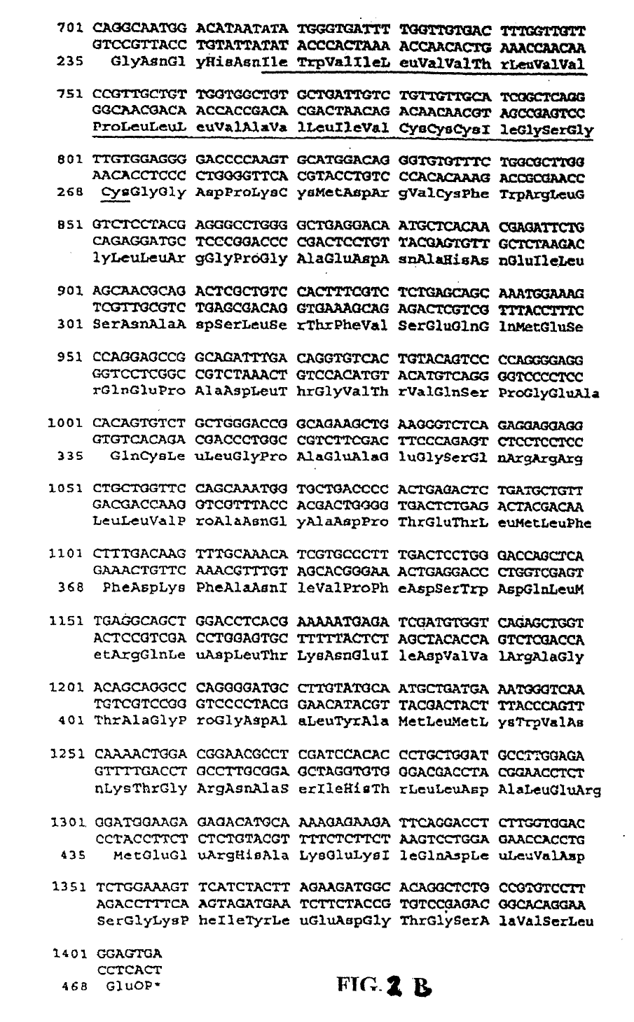

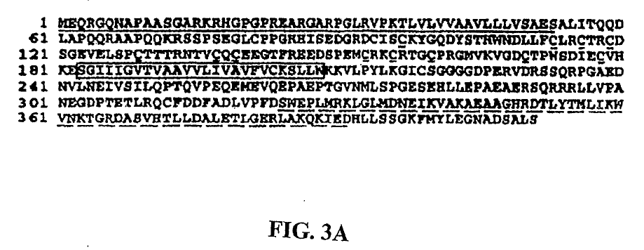

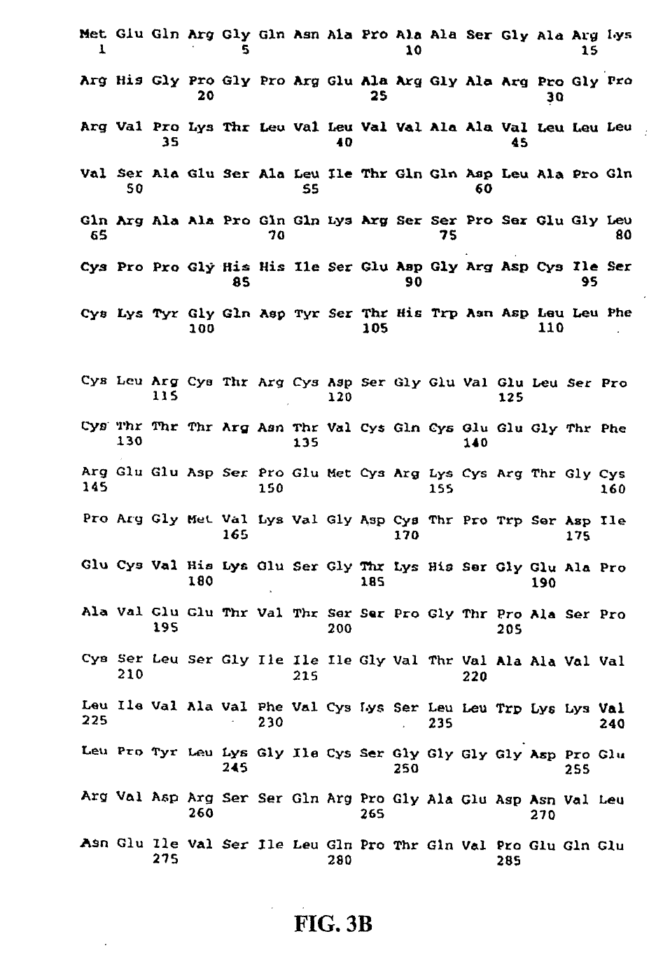

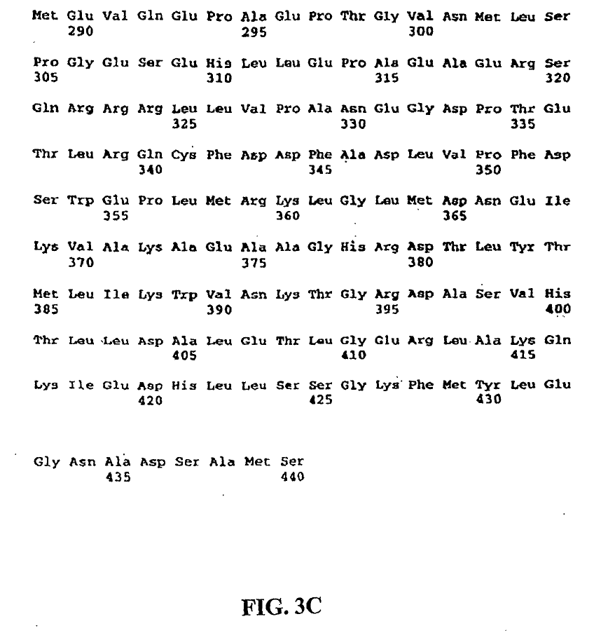

“Apo-2 ligand receptor” includes the receptors referred to in the art as “DR4” and “DR5” whose polynucleotide and polypeptide sequences are shown in FIGS. 2 and 3 respectively. Pan et al. have described the TNF receptor family member referred to as “DR4” (Pan et al., Science, 276:111-113 (1997); see also WO98/32856 published Jul. 30, 1998; WO 99/37684 published Jul. 29, 1999; WO 00/73349 published Dec. 7, 2000; U.S. Pat. No. 6,433,147 issued Aug. 13, 2002; U.S. Pat. No. 6,461,823 issued Oct. 8, 2002, and U.S. Pat. No. 6,342,383 issued Jan. 29, 2002). The DR4 receptor was reported to contain a cytoplasmic death domain capable of engaging the cell suicide apparatus. Pan et al. disclose that DR4 is believed to be a receptor for the ligand known as Apo2L/TRAIL. Sheridan et al., Science, 277:818-821 (1997) and Pan et al., Science, 277:815-818 (1997) described another receptor for Apo2L/TRAIL (see also, WO98/51793 published Nov. 19, 1998; WO98/41629 published Sep. 24, 1998). This receptor is referred to as DR5 (the receptor has also been alternatively referred to as Apo-2; TRAIL-R, TR6, Tango-63, hAPO8, TRICK2 or KILLER; Screaton et al., Curr. Biol., 7:693-696 (1997); Walczak et al., EMBO J., 16:5386-5387 (1997); Wu et al., Nature Genetics, 17:141-143 (1997); WO98/35986 published Aug. 20, 1998; EP870,827 published Oct. 14, 1998; WO98/46643 published Oct. 22, 1998; WO99/02653 published Jan. 21, 1999; WO99/09165 published Feb. 25, 1999; WO99/11791 published Mar. 11, 1999; US 2002/0072091 published Aug. 13, 2002; US 2002/0098550 published Dec. 7, 2001; U.S. Pat. No. 6,313,269 issued Dec. 6, 2001; US 2001/0010924 published Aug. 2, 2001; US 2003/01255540 published Jul. 3, 2003; US 2002/0160446 published Oct. 31, 2002, US 2002/0048785 published Apr. 25, 2002; U.S. Pat. No. 6,569,642 issued May 27, 2003; U.S. Pat. No. 6,072,047 issued Jun. 6, 2000; U.S. Pat. No. 6,642,358 issued Nov. 4, 2003). Like DR4, DR5 is reported to contain a cytoplasmic death domain and be capable of signaling apoptosis. As described above, other receptors for Apo-2L include DcR1, DcR2, and OPG (see, Sheridan et al., supra; Marsters et al., supra; and Simonet et al., supra). The term “Apo-2L receptor” when used herein encompasses native sequence receptor and receptor variants. These terms encompass Apo-2L receptor expressed in a variety of mammals, including humans. Apo-2L receptor may be endogenously expressed as occurs naturally in a variety of human tissue lineages, or may be expressed by recombinant or synthetic methods. A “native sequence Apo-2L receptor” comprises a polypeptide having the same amino acid sequence as an Apo-2L receptor derived from nature. Thus, a native sequence Apo-2L receptor can have the amino acid sequence of naturally-occurring Apo-2L receptor from any mammal. Such native sequence Apo-2L receptor can be isolated from nature or can be produced by recombinant or synthetic means. The term “native sequence Apo-2L receptor” specifically encompasses naturally-occurring truncated or secreted forms of the receptor (e.g., a soluble form containing, for instance, an extracellular domain sequence), naturally-occurring variant forms (e.g., alternatively spliced forms) and naturally-occurring allelic variants. Receptor variants may include fragments or deletion mutants of the native sequence Apo-2L receptor. FIG. 3A shows the 411 amino acid sequence of human DR5 as published in WO 98/51793 on Nov. 19, 1998. A transcriptional splice variant of human DR5 is known in the art. This DR5 splice variant encodes the 440 amino acid sequence of human DR5 shown in FIGS. 3B and 3C as published in WO 98/35986 on Aug. 20, 1998. Polypeptide sequences of DR5 and DR5 fusion proteins are also provided in Table 9 below.

The term “antagonist” is used in the broadest sense, and includes any molecule that partially or fully blocks, inhibits, or neutralizes one or more biological activities of Apo2L/TRAIL, DR4 or DR5, in vitro, in situ, or in vivo. Examples of such biological activities of Apo2L/TRAIL, DR4 or DR5 include binding of Apo2L/TRAIL to DR4 or DR5, induction of apoptosis as well as those further reported in the literature. An antagonist may function in a direct or indirect manner. For instance, the antagonist may function to partially or fully block, inhibit or neutralize one or more biological activities of Apo2L/TRAIL, in vitro, in situ, or in vivo as a result of its direct binding to DR4 or DR5. The antagonist may also function indirectly to partially or fully block, inhibit or neutralize one or more biological activities of Apo2L/TRAIL, DR4 or DR5, in vitro, in situ, or in vivo as a result of, e.g., blocking or inhibiting another effector molecule. The antagonist molecule may comprise a “dual” antagonist activity wherein the molecule is capable of partially or fully blocking, inhibiting or neutralizing a biological activity of Apo2L/TRAIL, DR4 or DR5.

The term “agonist” is used in the broadest sense, and includes any molecule that partially or fully enhances, stimulates or activates one or more biological activities of Apo2L/TRAIL, DR4 or DR5, in vitro, in situ, or in vivo. Examples of such biological activities binding of Apo2L/TRAIL to DR4 or DR5, apoptosis as well as those further reported in the literature. An agonist may function in a direct or indirect manner. For instance, the agonist may function to partially or fully enhance, stimulate or activate one or more biological activities of DR4 or DR5, in vitro, in situ, or in vivo as a result of its direct binding to DR4 or DR5, which causes receptor activation or signal transduction. The agonist may also function indirectly to partially or fully enhance, stimulate or activate one or more biological activities of DR4 or DR5, in vitro, in situ, or in vivo as a result of, e.g., stimulating another effector molecule which then causes DR4 or DR5 activation or signal transduction. It is contemplated that an agonist may act as an enhancer molecule which functions indirectly to enhance or increase DR4 or DR5 activation or activity. For instance, the agonist may enhance activity of endogenous Apo-2L in a mammal. This could be accomplished, for example, by pre-complexing DR4 or DR5 or by stabilizing complexes of the respective ligand with the DR4 or DR5 receptor (such as stabilizing native complex formed between Apo-2L and DR4 or DR5).

The term “tagged” when used herein refers to a chimeric molecule comprising an antibody or polypeptide fused to a “tag polypeptide”. The tag polypeptide has enough residues to provide an epitope against which an antibody can be made or to provide some other function, such as the ability to oligomerize (e.g. as occurs with peptides having leucine zipper domains), yet is short enough such that it generally does not interfere with activity of the antibody or polypeptide. The tag polypeptide preferably also is fairly unique so that a tag-specific antibody does not substantially cross-react with other epitopes. Suitable tag polypeptides generally have at least six amino acid residues and usually between about 8 to about 50 amino acid residues (preferably, between about 10 to about 20 residues).

The term “divalent metal ion” refers to a metal ion having two positive charges. Examples of divalent metal ions include but are not limited to zinc, cobalt, nickel, cadmium, magnesium, and manganese. Particular forms of such metals that may be employed include salt forms (e.g., pharmaceutically acceptable salt forms), such as chloride, acetate, carbonate, citrate and sulfate forms of the above mentioned divalent metal ions. Optionally, a divalent metal ion for use in the present invention is zinc, and preferably, the salt form, zinc sulfate or zinc chloride.

“Isolated,” when used to describe the various peptides or proteins disclosed herein, means peptide or protein that has been identified and separated and/or recovered from a component of its natural environment. Contaminant components of its natural environment are materials that would typically interfere with diagnostic or therapeutic uses for the peptide or protein, and may include enzymes, hormones, and other proteinaceous or non-proteinaceous solutes. In preferred embodiments, the peptide or protein will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain, or (3) to homogeneity by mass spectroscopic or peptide mapping techniques. Isolated material includes peptide or protein in situ within recombinant cells, since at least one component of its natural environment will not be present. Ordinarily, however, isolated peptide or protein will be prepared by at least one purification step.

“Percent (%) amino acid sequence identity” with respect to the sequences identified herein is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art can determine appropriate parameters for measuring alignment, including assigning algorithms needed to achieve maximal alignment over the full-length sequences being compared. For purposes herein, percent amino acid identity values can be obtained using the sequence comparison computer program, ALIGN-2, which was authored by Genentech, Inc. and the source code of which has been filed with user documentation in the US Copyright Office, Washington, D.C., 20559, registered under the US Copyright Registration No. TXU510087. The ALIGN-2 program is publicly available through Genentech, Inc., South San Francisco, Calif. All sequence comparison parameters are set by the ALIGN-2 program and do not vary.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired identity between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“High stringency conditions”, as defined herein, are identified by those that: (1) employ low ionic strength and high temperature for washing; 0.015 M sodium chloride/0.0015 M sodium citrate/0.1% sodium dodecyl sulfate at 50° C.; (2) employ during hybridization a denaturing agent; 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PEMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. Preferably, the cells express at least FcγRIII and carry out ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and Fcγ RIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)). FcRs herein include polymorphisms such as the genetic dimorphism in the gene that encodes FcγRIIIa resulting in either a phenylalanine (F) or a valine (V) at amino acid position 158, located in the region of the receptor that binds to IgG1. The honozygous valine FcγRIIIa (FcγRIIIa-158V) has been shown to have a higher affinity for human IgG1 and mediate increased ADCC in vitro relative to homozygous phenylalanine FcγRIIIa (FcγRIIIa-158F) or heterozygous (FcγRIIIa-158F/V) receptors.

“Complement dependent cytotoxicity” or “CDC” refer to the ability of a molecule to lyse a target in the presence of complement. The complement activation pathway is initiated by the binding of the first component of the complement system (Clq) to a molecule (e.g. an antibody) complexed with a cognate antigen. To assess complement activation, a CDC assay, e.g. as described in Gazzano-Santoro et al., J. Immunol. Methods 202:163 (1996), may be performed.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable or complementary determining regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cell-mediated cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of anti body, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, vol. 113, Rosenburg and Moore eds., Springer-Verlag, N.Y., pp. 269-315 (1994).

The term “diabodies“refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody which are responsible for antigen-binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (198)). “Framework” or “FR” residues are those variable domain residues other than the hypervariable region residues as herein defined.

An antibody “which binds” an antigen of interest, e.g. a DR5 receptor, is one capable of binding that antigen with sufficient affinity and/or avidity such that the antibody is useful as a therapeutic or diagnostic agent for targeting a cell expressing the antigen.

For the purposes herein, “immunotherapy” will refer to a method of treating a mammal (preferably a human patient) with an antibody, wherein the antibody may be an unconjugated or “naked” antibody, or the antibody may be conjugated or fused with heterologous molecule(s) or agent(s), such as one or more cytotoxic agent(s), thereby generating an “immunoconjugate”.

An “isolated” antibody is one which has been identified and separated and/or recovered from a component of its natural environment. Contaminant components of its natural environment are materials which would interfere with diagnostic or therapeutic uses for the antagonist or antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by, SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The expression “therapeutically effective amount” refers to an amount of the DR5 antibody which is effective for preventing, ameliorating or treating the disease or condition in question.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors; platelet-growth factor; transforming growth factors (TGFs) such as TGF-α and TGF-β; insulin-like growth factor-I and -II; erythropoietin (EPO); osteoinductive factors; interferons such as interferon-α, -β, and -gamma; colony stimulating factors (CSF) such as macrophage-CSF (M-CSF); granulocyte-macrophage-CSF (GM-CSF); and granulocyte-CSF (G-CSF); interleukins (ILs) such as IL-1, IL-2, IL-3, IL-4,IL-5, IL-6, IL-7, IL-8, IL-9, IL-11, IL-12, IL-13, IL-17; and other polypeptide factors including LIF and kit ligand (KL). As used herein, the term cytokine includes proteins from natural sources or from recombinant cell culture and biologically active equivalents of the native sequence cytokines.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolmelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1I and calicheamicin phiI1, see, e.g., Agnew, Chem Intl. Ed. Engl., 33:183-186 (1994); dynemicin, including dynemicin A; bisphosphonates, such as clodronate; an esperamicin; as well as neocarzinostatin chromophore and related chromoprotein enediyne antiobiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (Adriamycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rociorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; boestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhône-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitoxantrone; vincristine; vinorelbine (Navelbine™); novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in this definition are anti-hormonal agents that act to regulate or inhibit hormone action on tumors such as anti-estrogens and selective estrogen receptor modulators (SERMs), including, for example, tamoxifen (including Nolvadex™), raloxifene, droloexifene, 4-hydroxytamoxifen, trioxifene, keoxifene, LY117018, onapristone, and toremifene (Fareston™); aromatase inhibitors that inhibit the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprolide, and goserelin; and pharmaceutically acceptable salts, acids or derivatives of any of the above.

A “growth inhibitory agent” when used herein refers to a compound or composition which inhibits growth of a cell, especially cancer cell overexpressing any of the genes identified herein, either in vitro or in vivo. Thus, the growth inhibitory agent is one which significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al. (W B Saunders: Philadelphia, 1995), especially p. 13.

“Biologically active” or “biological activity” for the purposes herein means (a) having the ability to induce or stimulate or inhibit apoptosis in at least one type of mammalian cancer cell or virally-infected cell in vivo or ex vivo, either alone as a single agent or in combination with another agent such as a chemotherapeutic agent (b) capable of binding and/or stimulating a DR5 receptor; or (c) having some activity of a native or naturally-occurring Apo2L/TRAIL polypeptide. Assays for determining biological activity can be conducted using methods known in the art, such as DNA fragmentation (see, e.g., Marsters et al., Curr. Biology, 6: 1669 (1996)), caspase inactivation, DR5 binding (see, e.g., WO 98/51793, published Nov. 19, 1998), as well as the assays described in PCT Publication Nos. WO97/01633, WO97/25428, WO 01/00832, and WO 01/22987.

The terms “apoptosis” and “apoptotic activity” are used in a Broad sense and refer to the orderly or controlled form of cell death in mammals that is typically accompanied by one or more characteristic cell changes, including condensation of cytoplasm, loss of plasma membrane microvilli, segmentation of the nucleus, degradation of chromosomal DNA or loss of mitochondrial function. This activity can be determined and measured, for instance, by cell viability assays (such as Alamar blue assays or MTT assays), FACS analysis, caspase activation, DNA fragmentation (see, for example, Nicoletti et al., J. Immunol. Methods, 139:271-279 (1991), and poly-ADP ribose polymerase, “PARP”, cleavage assays known in the art.

As used herein, the term “disorder” in general refers to any condition that would benefit from treatment with the compositions described herein, including any disease or disorder that can be treated by effective amounts of a DR5 antibody. This includes chronic and acute disorders, as well as those pathological conditions which predispose the mammal too the disorder in question. Non-limiting examples of disorders to be treated herein include benign and malignant cancers; inflammatory, angiogenic, and immunologic disorders, autoimmune disorders, arthritis (including rheumatoid arthritis), multiple sclerosis, and HIV/AIDS.

The terms “cancer,” “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, gastrointestinal cancer, renal cancer, ovarian cancer, liver cancer, lymphoblastic leukemia, lymphocytic leukemia, colorectal cancer, endometrial cancer, kidney cancer, prostate cancer, thyroid cancer, neuroblastoma, pancreatic cancer, glioblastoma multiforme, cervical cancer, brain cancer, stomach cancer, bladder cancer, hepatoma, breast cancer, colon carcinoma, and head and neck cancer.

The term “immune related disease” means a disease in which a component of the immune system of a mammal causes, mediates or otherwise contributes to morbidity in the mammal. Also included are diseases in which stimulation or intervention of the immune response has an ameliorative effect on progression of the disease. Included within this term are autoimmune diseases, immune-mediated inflammatory diseases, non-immune-mediated inflammatory diseases, infectious diseases, and immunodeficiency diseases. Examples of immune-related and inflammatory diseases, some of which are immune or T cell mediated, which can be treated according to the invention include systemic lupus erythematosis, rheumatoid arthritis, juvenile chronic arthritis, spondyloarthropathies, systemic sclerosis (scleroderma), idiopathic inflammatory myopathies (dermatomyositis, pblymyositis), Sjogren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory and fibrotic lung diseases such as inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases include AIDS (HIV infection), hepatitis A, B, C, D, and E, bacterial infections, fungal infections, protozoal infections and parasitic infections.

“Autoimmune disease” is used herein in a broad, general sense to refer to disorders or conditions in mammals in which destruction of normal or healthy tissue arises from humoral or cellular immune responses of the individual mammal to his or her own tissue constituents. Examples include, but are not limited to, lupus erythematous, thyroiditis, rheumatoid arthritis, psoriasis, multiple sclerosis, autoimmune diabetes, and inflammatory bowel disease (IBD).

The terms “treating”, “treatment” and “therapy” as used herein refer to curative therapy, prophylactic therapy, and preventative therapy. Consecutive treatment or administration refers to treatment on at least a daily basis without interruption in treatment by one or more days. Intermittent treatment or administration, or treatment or administration in an intermittent fashion, refers to treatment that is not consecutive, but rather cyclic in nature.

The term “mammal” as used herein refers to any mammal classified as a mammal, including humans, cows, horses, dogs and cats. In a preferred embodiment of the invention, the mammal is a human.

In this application, the use of the singular includes the plural unless specifically stated otherwise.

### B. Exemplary Materials and Methods of the Invention

The invention described herein relates to antibodies that bind to DR5 receptor. Optionally the antibody is an antagonist which inhibits the interaction of Apo-2L with DR5. Alternatively, the antibody is an agonist of DR5 signalling activity.

Methods for generating DR5 antibodies of the invention are described herein. The antigen to be used for production of, or screening for, antibody may be, e.g., a soluble form of the antigen or a portion thereof, containing the desired epitope. Alternatively, or additionally, cells expressing the antigen at their cell surface can be used to generate, or screen for, antibody. Other forms of the antigen useful for generating antibody will be apparent to those skilled in the art.

(i) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies. Further phage display techniques for identifying DR5 antibodies of the invention are described in additional detail in the Examples section below.

In certain embodiments, the complementarity determining regions (CDRs) of the light and heavy chain variable regions may be grafted to framework regions (FRs) from the same, or another, species. In certain embodiments, the CDRs of the light and heavy chain variable regions may be grafted to consensus human FRs. To create consensus human FRs, in certain embodiments, FRs from several human heavy chain or light chain amino acid sequences are aligned to identify a consensus amino acid sequence. In certain embodiments, the grafted variable regions may be used with a constant region that is different from the constant region of the source antibody. In certain embodiments, the grafted variable regions are part of a single chain Fv antibody. CDR grafting is described, e.g., in U.S. Pat. Nos. 6,180,370, 5,693,762, 5,693,761, 5,585,089, and 5,530,101.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

(vi) Bispecific Antibodies

Bispecific antibodies are antibodies that have binding specificities for at least two different epitopes. Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985); Shalaby et al., J. Exp. Med., 175: 217-225 (1992).

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991). Antibodies with three or more antigen binding sites are described in WO01/77342 (Miller and Presta), expressly incorporated herein by reference.

The antibody used in the methods or included in the articles of manufacture herein is optionally conjugated to a cytotoxic agent.

Chemotherapeutic agents useful in the generation of such antibody-cytotoxic agent conjugates have been described above.

Conjugates of an antibody and one or more small molecule toxins, such as a calicheamicin, a maytansine (U.S. Pat. No. 5,208,020), a trichothene, and CC1065 are also contemplated herein. In one embodiment of the invention, the antibody is conjugated to one or more maytansine molecules (e.g. about 1 to about 10 maytansine molecules per antibody molecule). Maytansine may, for example, be converted to May-SS-Me which may be reduced to May-SH3 and reacted with modified antibody (Chari et al. Cancer Research 52: 127-131 (1992)) to generate a maytansinoid-antibody conjugate.

Alternatively, the antibody is conjugated to one or more calicheamicin molecules. The calicheamicin family of antibiotics is capable of producing double-stranded DNA breaks at sub-picomolar concentrations. Structural analogues of calicheamicin which may be used include, but are not limited to, γ₁^{I}, α₂^{I}, α₃^{I}, N-acetyl-γ₁^{I}, PSAG and θ^{I}₁ (Hinman et al. Cancer Research 53: 3336-3342 (1993) and Lode et al. Cancer Research 58: 2925-2928 (1998)).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, cretin, sapaonaria officinalis inhibitor, gelonin, mitogellin, restrictocin, phenomycin, enomycin and the tricothecenes. See, for example, WO 93/21232 published Oct. 28, 1993.

The present invention further contemplates antibody conjugated with a compound with nucleolytic activity (e.g. a ribonuclease or a DNA endonuclease such as a deoxyribonuclease; DNase).

A variety of radioactive isotopes are available for the production of radioconjugated antagonists or antibodies. Examples include At²¹¹, I¹³¹, I¹²⁵, Y⁹⁰, Re¹⁸⁶, Re¹⁸⁸, Sm¹⁵³, Bi²¹², P³² and radioactive isotopes of Lu.

Conjugates of the antibody and cytotoxic agent may be made using a variety of bifunctional protein coupling agents such as N-succinimidyl-3-(2-pyridyldithiol)propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MK-DTPA) is an exemplary chelating agent for conjugation of radionucleotide to the antagonist or antibody. See WO94/11026. The linker may be a “cleavable linker” facilitating release of the cytotoxic drug in the cell. For example, an acid-labile linker, peptidase-sensitive linker, climethyl linker or disulfide-containing linker (Chari et al. Cancer Research 52: 127-131 (1992)) may be used.

Alternatively, a fusion protein comprising the antibody and cytotoxic agent may be made, e.g. by recombinant techniques or peptide synthesis.

The antibodies of the present invention may also be conjugated with a prodrug-activating enzyme which converts a prodrug (e.g. a peptidyl chemotherapeutic agent, see WO81/01145) to an active anti-cancer drug. See, for example, WO 88/07378 and U.S. Pat. No. 4,975,278.

The enzyme component of such conjugates includes any enzyme capable of acting on a prodrug in such a way so as to covert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-flucrocytosine into the anti-cancer drag, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). Antibody-abzyme conjugates can be prepared as described herein for delivery of the abzyme to a tumor cell population.

The enzymes of this invention can be covalently bound to the antibody by techniques well known in the art such as the use of the heterobifunctional crosslinking reagents discussed above. Alternatively, fusion proteins comprising at least the antigen binding region of an antibody linked to at least a functionally active portion of an enzyme of the invention can be constructed using recombinant DNA techniques well known in the art (see, e.g., Neuberger et al., Nature, 312: 604-608 (1984)).

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol.

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Alternatively, or additionally, one may increase, or decrease, serum half-life by altering the amino acid sequence of the Fc region of an antibody to generate variants with altered FcRn binding. Antibodies with altered FcRn binding and/or serum half life are described in WO00/42072 (Presta, L.).

The antibodies of the invention may be stabilized by polymerization. This may be accomplished by crosslinking monomer chains with polyfunctional crosslinking agents, either directly or indirectly, through multi-functional polymers. Ordinarily, two substantially identical polypeptides are crosslinked at their C- or N-termini using a bifunctional crosslinking agent. The agent is used to crosslink the terminal amino and/or carboxyl groups. Generally, both terminal carboxyl groups or both terminal amino groups are crosslinked to one another, although by selection of the appropriate crosslinking agent the alpha amino of one polypeptide is crosslinked to the terminal carboxyl group of the other polypeptide. Preferably, the polypeptides are substituted at their C-termini with cysteine. Under conditions well known in the art a disulfide bond can be formed between the terminal cysteines, thereby crosslinking the polypeptide chains. For example, disulfide bridges are conveniently formed by metal-catalyzed oxidation of the free cysteines or by nucleophilic substitution of a suitably modified cysteine residue. Select ion of the crosslinking agent will depend upon the identities of the reactive side chains of the amino acids present in the polypeptides. For example, disulfide crosslinking would not be preferred if cysteine was present in the polypeptide at additional sites other than the C-terminus. Also within the scope hereof are peptides crosslinked with methylene bridges.

Suitable crosslinking sites on the antibodies, aside from the N-terminal amino and C-terminal carboxyl groups, include epsilon amino groups found on lysine residues, as well as amino, imino, carboxyl, sulfhydryl and hydroxyl groups located on the side chains of internal residues of the peptides or residues introduced into flanking sequences. Crosslinking through externally added crosslinking agents is suitably achieved, e.g., using any of a number of reagents familiar to those skilled in the art, for example, via carbodiimide treatment of the polypeptide. Other examples of suitable multi-functional (ordinarily bifunctional) crosslinking agents are found in the literature.

### C. Preparation of Typical Formulations of the Invention

In the preparation of typical formulations herein, it is noted that the recommended quality or “grade” of the components employed will depend on the ultimate use of the formulation. For therapeutic uses, it is preferred that the component(s) are of an allowable grade (such as “GRAS”) as an additive to pharmaceutical products.

In certain embodiments, there are provided compositions comprising DR5 receptor antibody(s) and one or more excipients which provide sufficient ionic strength to enhance solubility and/or stability of the antibodies, wherein the composition has a pH of 6 (or about 6) to 9 (or about 9). The antibody may be prepared by any suitable method to achieve the desired purity of the protein, for example, according to the above methods. In certain embodiments, the DR5 antibody is recombinantly expressed in host cells or prepared by chemical synthesis. The concentration of the antibody in the formulation may vary depending, for instance, on the intended use of the formulation. Those skilled in the art can determine without undue experimentation the desired concentration of the DR5 antibody.

The one or more excipients in the formulations which provide sufficient ionic strength to enhance solubility and/or stability of the DR5 antibody is optionally a polyionic organic or inorganic acid, aspartate, sodium sulfate, sodium succinate, sodium acetate, sodium chloride, Captisol™, Tris, arginine salt or other amino acids, sugars and polyols such as trehalose and sucrose. Preferably the one or more excipients in the formulations which provide sufficient ionic strength is a salt. Salts which may be employed include but are not limited to sodium salts and arginine salts. The type of salt employed and the concentration of the salt are preferably such that the formulation has a relatively high ionic strength which allows the DR5 antibody in the formulation to be stable. Optionally, the salt is present in the formulation at a concentration of about 20 mM to about 0.5 M.

The composition preferably has a pH of 6 (or about 6) to 9 (or about 9), more preferably about 6.5 to about 8.5, and even more preferably about 7 to about 7.5. In a preferred aspect of this embodiment, the composition will further comprise a buffer to maintain the pH of the composition at least about 6 to about 8. Examples of buffers which may be employed include but are not limited to Tris, HEPES, and histidine. When employing Tris, the pH may optionally be adjusted to about 7 to 8.5. When employing Hepes or histidine, the pH may optionally be adjusted to about 6.5 to 7. Optionally, the buffer is employed at a concentration of about 5 mM to about 50 mM in the formulation.

Particularly for liquid formulations (or reconstituted lyophilized formulations), it may be desirable to include one or more surfactants in the composition. Such surfactants may, for instance, comprise a non-ionic surfactant like TWEEN™ or PLURONICS™ (e.g., polysorbate or poloxamer). Preferably, the surfactant comprises polysorbate 20 (“Tween 20”). The surfactant will optionally be employed at a concentration of about 0.005% to about 0.2%.

The formulations of the present invention may include, in addition to DR5 antibody(s) and those components described above, further various other excipients or components. Optionally, the formulation may contain, for parenteral administration, a pharmaceutically or parenterally acceptable carrier, i.e., one that is non-toxic to recipients at the dosages and concentrations employed and is compatible with other ingredients of the formulation. Optionally, the carrier is a parenteral carrier, such as a solution that is isotonic with the blood of the recipient. Examples of such carrier vehicles include water, saline or a buffered solution such as phosphate-buffered saline (PBS), Ringer's solution, and dextrose solution. Various optional pharmaceutically acceptable carriers, excipients, or stabilizers are described further in Remington's Pharmaceutical Sciences, 16th edition, Osol, A. ed. (1980).

The formulations herein also may contain one or more preservatives. Examples include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols, alkyl parabens such as methyl or propyl paraben, and m-cresol. Antioxidants include ascorbic acid and methionine; preservatives (such as octadecyldimethylbenzyl ammonium chloride; hexamethonium chloride; benzalkonium chloride, benzethonium chloride; butyl alcohol; alkyl parabens such as methyl or propyl paraben; catechol; resorcinol; cyclohezanol; 3-pentanol; and m-cresol); low molecular weight (less than about 10 residues) polypeptides; proteins, such as serum albumin, gelatin, or immunoflobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; or polyethylene glycol (PEG).

Additional examples of such carriers include lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, sodium chloride, polyvinyl pyrrolidone, and cellulose-based substances. Carriers for gel-based forms include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. Conventional depot forms include, for example, microcapsules, nano-capsules, liposomes, plasters, inhalation forms, nose sprays, and sustained-release preparations.

The compositions of the invention may comprise liquid formulations (liquid solutions or liquid suspensions), and lyophilized formulations, as well as suspension formulations in which the DR5 antibody is in the form of crystals or amorphous precipitate.

The final formulation, if a liquid, is preferably stored frozen at ≦20° C. Alternatively, the formulation can be lyophilized and provided as a powder for reconstitution with water for injection that optionally may be stored at 2-30° C.

The formulation to be used for therapeutic administration must be sterile. Sterility is readily accomplished by filtration through sterile filtration membranes (e.g., 0.2 micron membranes). Therapeutic compositions generally are placed into a container having a sterile access port, for example, an intravenous solution bag or vial having a stopper pierceable by a hypodermic injection needle.

The composition ordinarily will be stored in single unit or multi-dose containers, for example, sealed ampules or vials, as an aqueous solution or as a lyophilized formulation for reconstitution. The containers may any available containers in the art and filled using conventional methods. Optionally, the formulation may be included in an injection pen device (or a cartridge which fits into a pen device), such as those available in the art (see, e.g., U.S. Pat. No. 5,370,629), which are suitable for therapeutic delivery of the formulation. An injection solution can be prepared by reconstituting the lyophilized DR5 antibody formulation using, for example, Water-for-Injection.

### D. Methods of Use and Other Applications

The DR5 antibodies described herein can be employed in a variety of therapeutic and non-therapeutic applications. Among these applications are methods of treating disorders, such as cancer, immune related conditions, or viral conditions. Such therapeutic and non-therapeutic applications are further described, for instance, in WO97/25428, WO97/01633, and WO 01/22987.

The invention contemplates using gene therapy for treating a mammal, using nucleic acid encoding the DR5 antibody. Nucleic acids which encode the DR5 antibody can be used for this purpose. Once the amino acid sequence is known, one can generate several nucleic acid molecules using the degeneracy of the genetic code, and select which to use for gene therapy.

There are two major approaches to getting the nucleic acid (optionally contained in a vector) into the patient's cells for purposes of gene therapy: in vivo and ex vivo. For in vivo delivery, the nucleic acid is injected directly into the patient, usually at the site where the DR5 antibody is required. For ex vivo treatment, the patient's cells are removed, the nucleic acid is introduced into these isolated cells and the modified cells are administered to the patient either directly or, for example, encapsulated within porous membranes which are implanted into the patient. See, e.g. U.S. Pat. Nos. 4,892,538 and 5,283,187.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem., 262: 4429-4432 (1987); and Wagner et al., Proc. Natl. Acad. Sci. USA, 87: 3410-3414 (1990). For review of the currently known gene marking and gene therapy protocols, see Anderson et al., Science, 256: 808-813 (1992). See also WO 93/25673 and the references cited therein.

In the methods of the invention for treating a disorder, a formulation of DR5 antibody can be directly administered to the mammal by any suitable technique, including infusion or injection. The specific route of administration will depend, e.g., on the medical history of the patient, including any perceived or anticipated side effects using DR5 antibody and the particular disorder to be corrected. Examples of parenteral administration include subcutaneous, intramuscular, intravenous, intraarterial, and intraperitoneal administration of the composition. The formulations are preferably administered as repeated intravenous (i.v.), subcutaneous (s.c.), intramuscular (i.m.) injections or infusions, intracranial infusions or as aerosol formulations suitable for intranasal or intrapulmonary delivery (for intrapulmonary delivery see, e.g., EP 257,956).

It is noted that osmotic pressure of injections may be important in subcutaneous and intramuscular injection. Injectable solutions, when hypotonic or hypertonic, may cause pain to a patient upon infusion. Usually, for the therapeutic, injectable formulations herein, it is preferred that the relative osmolarity of the injectable solution be about 300 mosm to about 600 mosm.

DR5 antibody formulations can also be administered in the form of oral or sustained-release preparations. Suitable examples of sustained-release preparations include semipermeable matrices of solid hydrophobic polymers containing the protein, which matrices are in the form of shaped articles, e.g., films, or microcapsules. Examples of sustained-release matrices include cellulose derivatives (e.g., carboxymethylcellulose), sucrose-acetate isobutyrate (SABER™) in non-aqueous media, polyesters, hydrogels (e.g., poly(2-hydroxyethyl-methacrylate) (Langer et al., J. Biomed. Mater. Res. 1981, 15: 167-277; Langer, Chem. Tech. 1982, 12: 98-105 or poly(vinylalcohol)), polylactides (U.S. Pat. No. 3,773,919, EP 58,481), copolymers of L-glutamic acid and gamma ethyl-L-glutamate (Sidman et al., Biopolymers 1983, 22: 547-556), non-degradable ethylene-vinyl acetate (Langer et al., supra), degradable lactic acid-glycolic acid copolymers such as the Lupron Depot (injectable microspheres composed of lactic acid-glycolic acid copolymer and leuprolide acetate), and poly-D-(−)-3-hydroxybutyric acid (EP 133,988). One optional method of delivery for systemic-acting drugs involves administration by continuous infusion (using, e.g., slow-release devices or minipumps such as osmotic pumps or skin patches), or by injection (using, e.g., intravenous or subcutaneous means, including single-bolus administration).

The composition to be used in the therapy will be formulated and dosed in a fashion consistent with good medical practice, taking into account the clinical condition of the individual patient, the site of delivery of the composition, the method of administration, the scheduling of administration, and other factors known to practitioners.

It is contemplated that yet additional therapies may be employed in the methods. The one or more other therapies may include but are not limited to, administration of radiation therapy, cytokine(s), growth inhibitory agent(s), chemotherapeutic agent(s), cytotoxic agent(s), tyrosine kinase inhibitors, ras farnesyl transferase inhibitors, angiogenesis inhibitors, and cyclin-dependent kinase inhibitors which are known in the art and defined further with particularity above, and may be administered in combination (e.g., concurrently or sequentially) with DR5 antibody. In addition, therapies based on therapeutic antibodies that target tumor or other cell antigens such as CD20 antibodies (including Rituxan™) or Her receptor antibodies (including Herceptin™) as well as anti-angiogenic antibodies such as anti-VEGF, or antibodies that target other Apo2L receptors, such as DR4.

Preparation and dosing schedules for chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). In some instances, it may be beneficial to expose cells to one or more chemotherapeutic agents prior to administering DR5 antibody. By way of example, some types of cancer cells may be resistant to apoptosis-induction by a DR5 antibody, but can become sensitive to such a DR5 antibody by pre-treating the cells with a chemotherapeutic agent.

It may be desirable to also administer antibodies against other antigens, such as antibodies which bind to CD20, CD11a, CD18, CD40, ErbB2, EGFR, ErbB3, ErbB4, vascular endothelial factor (VEGF), or other TNFR family members (such as OPG, DR4, TNFR1, TNFR2). Alternatively, or in addition, two or more antibodies binding the same or two or more different antigens disclosed herein may be co-administered to the patient. Sometimes, it may be beneficial to also administer one or more cytokines to the patient.

The DR5 antibody formulation may be administered in any of the therapeutic methods described in this application in combination with, e.g., concurrently or sequentially, with other agents, cytokines, chemotherapies, antibodies, etc. that are for example, specifically provided in the Definition section of the application above. For example, the DR5 antibody formulation may be administered as a pre-treatment (prior to administration of any such other agents), such as a pre-treatment of cancer cells which may otherwise be resistant to the apoptotic effects of other therapeutic agents.

As noted above, DR5 antibodies of the invention have various utilities. For example, DR5 agonistic peptides may be employed in methods for treating pathological conditions in mammals such as cancer or immune-related diseases. Diagnosis in mammals of the various pathological conditions described herein can be made by the skilled practitioner. Diagnostic techniques are available in the art which allow, e.g., for the diagnosis or detection of cancer or immune related disease in a mammal. For instance, cancers may be identified through techniques, including but not limited to, palpation, blood analysis, x-ray, NMR and the like. Immune related diseases can also be readily identified. In systemic lupus erythematosus, the central mediator of disease is the production of auto-reactive antibodies to self proteins/tissues and the subsequent generation of immune-mediated inflammation. Multiple organs and systems are affected clinically including kidney, lung, musculoskeletal system, mucocutaneous, eye, central nervous system, cardiovascular system, gastrointestinal tract, bone marrow and blood.

Medical practitioners are familiar with a number diseases in which intervention of the immune and/or inflammatory response have benefit. For example, rheumatoid arthritis (RA) is a chronic systemic autoimmune inflammatory disease that mainly involves the synovial membrane of multiple joints with resultant injury to the articular cartilage. The pathogenesis is T lymphocyte dependent and is associated with the production of rheumatoid factors, auto-antibodies directed against self IgG, with the resultant formation of immune complexes that attain high levels in joint fluid and blood. These complexes in the joint may induce the marked infiltrate of lymphocytes and monocytes into the synovium and subsequent marked synovial changes; the joint space/fluid if infiltrated by similar cells with the addition of numerous neutrophils. Tissues affected are primarily the joints, often in symmetrical pattern. However, extra-articular disease also occurs in two major forms. One form is the development of extra-articular lesions with ongoing progressive joint disease and typical lesions of pulmonary fibrosis, vasculitis, and cutaneous ulcers. The second form of extra-articular disease is the so called Felty's syndrome which occurs late in the RA disease course, sometimes after joint disease has become quiescent, and involves the presence of neutropenia, thrombocytopenia and splenomegaly. This can be accompanied by vasculitis in multiple organs with formations of infarcts, skin ulcers and gangrene. Patients often also develop rheumatoid nodules in the subcutis tissue overlying affected joints; the nodules late stage have necrotic centers surrounded by a mixed inflammatory cell infiltrate. Other manifestations which can occur in RA include: pericarditis, pleuritis, coronary arteritis, interstitial pneumonitis with pulmonary fibrosis, keratoconjunctivitis sicca, and rheumatoid nodules.

Juvenile chronic arthritis is a chronic idiopathic inflammatory disease which begins often at less than 16 years of age. Its phenotype has some similarities to RA; some patients which are rheumatoid factor positive are classified as juvenile rheumatoid arthritis. The disease is sub-classified into three major categories: pauciarticular, polyarticular, and systemic. The arthritis can be severe and is typically destructive and leads to joint ankylosis and retarded growth. Other manifestations can include chronic anterior uveitis and systemic amyloidosis.

Spondyloarthropathies are a group of disorders with some common clinical features and the common association with the expression of HLA-B27 gene product. The disorders include: ankylosing spondylitis, Reiter's syndrome (reactive arthritis), arthritis associated with inflammatory bowel disease, spondylitis associated with psoriasis, juvenile onset spondyloarthropathy and undifferentiated spondyloarthropathy. Distinguishing features include sacroileitis with or without spondylitis; inflammatory asymmetric arthritis; association with HLA-B27 (a serologically defined allele of the HLA-B locus of class I MHC); ocular inflammation, and absence of autoantibodies associated with other rheumatoid disease. The cell most implicated as key to induction of the disease is the CD8+ T lymphocyte, a cell which targets antigen presented by class I MHC molecules. CD8+ T cells may react against the class I MHC allele HLA-B27 as if it were a foreign peptide expressed by MHC class I molecules. It has been hypothesized that an epitope of HLA-B27 may mimic a bacterial or other microbial antigenic epitope and thus induce a CD8+ T cells response.

Systemic sclerosis (scleroderma) has an unknown etiology. A hallmark of the disease is induration of the skin; likely this is induced by an active inflammatory process. Scleroderma can be localized or systemic; vascular lesions are common and endothelial cell injury in the microvasculature is an early and important event in the development of systemic sclerosis; the vascular injury may be immune mediated. An immunologic basis is implied by the presence of mononuclear cell infiltrates in the cutaneous lesions and the presence of anti-nuclear antibodies in many patients. ICAM-1 is often upregulated on the cell surface of fibroblasts in skin lesions suggesting that T cell interaction with these cells may have a role in the pathogenesis of the disease. Other organs involved include: the gastrointestinal tract: smooth muscle atrophy and fibrosis resulting in abnormal peristalsis/motility; kidney: concentric subendothelial intimal proliferation affecting small arcuate and interlobular arteries with resultant reduced renal cortical blood flow, results in proteinuria, azotemia and hypertension; skeletal muscle: atrophy, interstitial fibrosis; inflammation; lung: interstitial pneumonitis and interstitial fibrosis; and heart: contraction band necrosis, scarring/fibrosis.

Idiopathic inflammatory myopathies including dermatomyositis, polymyositis and others are disorders of chronic muscle inflammation of unknown etiology resulting in muscle weakness. Muscle injury/inflammation is often symmetric and progressive. Autoantibodies are associated with most forms. These myositis-specific autoantibodies are directed against and inhibit the function of components, proteins and RNA's, involved in protein synthesis.

Sjogren's syndrome is due to immune-mediated inflammation and subsequent functional destruction of the tear glands and salivary glands. The disease can be associated with or accompanied by inflammatory connective tissue diseases. The disease is associated with autoantibody production against Ro and La antigens, both of which are small RNA-protein complexes. Lesions result in keratoconjunctivitis sicca, xerostomia, with other manifestations or associations including bilary cirrhosis, peripheral or sensory neuropathy, and palpable purpura.

Systemic vasculitis are diseases in which the primary lesion is inflammation and subsequent damage to blood vessels which results in ischemia/necrosis/degeneration to tissues supplied by the affected vessels and eventual end-organ dysfunction in some cases. Vasculitides can also occur as a secondary lesion or sequelae to other immune-inflammatory mediated diseases such as rheumatoid arthritis, systemic sclerosis, etc., particularly in diseases also associated with the formation of immune complexes. Diseases in the primary systemic vasculitis group include: systemic necrotizing vasculitis: polyarteritis nodosa, allergic angiitis and granulomatosis, polyangiitis; Wegener's granulomatosis; lymphomatoid granulomatosis; and giant cell arteritis. Miscellaneous vasculitides include: mucocutaneous lymph node syndrome (MLNS or Kawasaki's disease), isolated CNS vasculitis, Behet's disease, thromboangiitis obliterans (Buerger's disease) and cutaneous necrotizing venulitis. The pathogenic mechanism of most of the types of vasculitis listed is believed to be primarily due to the deposition of immunoglobulin complexes in the vessel wall and subsequent induction of an inflammatory response either via ADCC, complement activation, or both.

Sarcoidosis is a condition of unknown etiology which is characterized by the presence of epithelioid granulomas in nearly any tissue in the body; involvement of the lung is most common. The pathogenesis involves the persistence of activated macrophages and lymphoid cells at sites of the disease with subsequent chronic sequelae resultant from the release of locally and systemically active products released by these cell types.

Autoimmune hemolytic anemia including autoimmune hemolytic anemia, immune pancytopenia, and paroxysmal noctural hemoglobinuria is a result of production of antibodies that react with antigens expressed on the surface of red blood cells (and in some cases other blood cells including platelets as well) and is a reflection of the removal of those antibody coated cells via complement mediated lysis and/or ADCC/Fc-receptor-mediated mechanisms.

In autoimmune thrombocytopenia including thrombocytopenic purpura, and immune-mediated thrombocytopenia in other clinical settings, platelet destruction/removal occurs as a result of either antibody or complement attaching to platelets and subsequent removal by complement lysis, ADCC or FC-receptor mediated mechanisms.

Thyroiditis including Gravels disease, Hashimoto's thyroiditis, juvenile lymphocytic thyroiditis, and atrophic thyroiditis, are the result of an autoimmune response against thyroid antigens with production of antibodies that react with proteins present in and often specific for the thyroid gland. Experimental models exist including spontaneous models: rats (BUF and BB rats) and chickens (obese chicken strain); inducible models: immunization of animals with either thyroglobulin, thyroid microsomal antigen (thyroid peroxidase).

Type I diabetes mellitus or insulin-dependent diabetes is the autoimmune destruction of pancreatic islet β cells; this destruction is mediated by auto-antibodies and auto-reactive T cells. Antibodies to insulin or the insulin receptor can also produce the phenotype of insulin-non-responsiveness.

Immune mediated renal diseases, including glomerulonephritis and tubulointerstitial nephritis, are the result of antibody or T lymphocyte mediated injury to renal tissue either directly as a result of the production of autoreactive antibodies or T cells against renal antigens or indirectly as a result of the deposition of antibodies and/or immune complexes in the kidney that are reactive against other, non-renal antigens. Thus other immune-mediated diseases that result in the formation of immune-complexes can also induce immune mediated renal disease as an indirect sequelae. Both direct and indirect immune mechanisms result in inflammatory response that produces/induces lesion development in renal tissues with resultant organ function impairment and in some cases progression to renal failure. Both humoral and cellular immune mechanisms can be involved in the pathogenesis of lesions.

Demyelinating diseases of the central and peripheral nervous systems, including Multiple Sclerosis; idiopathic demyelinating polyneuropathy or Guillain-Barr syndrome; and Chronic Inflammatory Demyelinating Polyneuropathy, are believed to have an autoimmune basis and result in nerve demyelination as a result of damage caused to oligodendrocytes or to myelin directly. In MS there is evidence to suggest that disease induction and progression is dependent on T lymphocytes. Multiple Sclerosis is a demyelinating disease that is T lymphocyte-dependent and has either a relapsing-remitting course or a chronic progressive course. The etiology is unknown; however, viral infections, genetic predisposition, environment, and autoimmunity all contribute. Lesions contain infiltrates of predominantly T lymphocyte mediated, microglial cells and infiltrating macrophages; CD4+T lymphocytes are the predominant cell type at lesions. The mechanism of oligodendrocyte cell death and subsequent demyelination is not known but is likely T lymphocyte driven.

Inflammatory and Fibrotic Lung Disease, including Eosinophilic Pneumonias; Idiopathic Pulmonary Fibrosis, and Hypersensitivity Pneumonitis may involve a disregulated immune-inflammatory response. Inhibition of that response would be of therapeutic benefit.

Autoimmune or Immune-mediated Skin Disease including Bullous Skin Diseases, Erythema Multiforme, and Contact Dermatitis are mediated by auto-antibodies, the genesis of which is T lymphocyte-dependent.

Psoriasis is a T lymphocyte-mediated inflammatory disease. Lesions contain infiltrates of T lymphocytes, macrophages and antigen processing cells, and some neutrophils.

Allergic diseases, including asthma; allergic rhinitis; atopic dermatitis; food hypersensitivity; and urticaria are T lymphocyte dependent. These diseases are predominantly mediated by T lymphocyte induced inflammation, IgE mediated-inflammation or a combination of both.

Transplantation associated diseases, including Graft rejection and Graft-Versus-Host-Disease (GVED) are T lymphocyte-dependent; inhibition of T lymphocyte function is ameliorative.

Other diseases in which intervention of the immune and/or inflammatory response have benefit are Infectious disease including but not limited to viral infection (including but not limited to AIDS, hepatitis A, B, C, D, E) bacterial infection, fungal infections, and protozoal and parasitic infections (molecules (or derivatives/agonists) which stimulate the MLR can be utilized therapeutically to enhance the immune response to infectious agents), diseases of immunodeficiency (molecules/derivatives/agonists) which stimulate the MLR can be utilized therapeutically to enhance the immune response for conditions of inherited, acquired, infectious induced (as in HIV infection), or iatrogenic (i.e. as from chemotherapy) immunodeficiency), and neoplasia.

Diagnostic methods are also provided herein. For instance, the DR5 antibodies may be employed to detect the respective DR5 receptors in mammals known to be or suspected of having a Apo-2L or DR5 related pathological condition. The binding peptides may be used, e.g., in assays to detect or quantitate DR5 in a sample. A sample, such as cells obtained from a mammal, can be incubated in the presence of a labeled binding peptide, and detection of the labeled binding peptide bound in the sample can be performed. Such assays, including various clinical assay procedures, are known in the art, for instance as described in Voller et al., Immunoassays, University Park, 1981.

The invention also provides kits which include DR5 antibodies described herein. A typical kit will comprise a container, preferably a vial, for DR5 antibody in one or more excipients as described above; and instructions, such as a product insert or label, directing the user as to how to employ the DR5 antibody formulation. This would preferably provide a pharmaceutical formulation. Preferably, the pharmaceutical formulation is for treating cancer or an immune related condition. Suitable containers include, for example, bottles, vials, syringes, and test tubes. The containers may be formed from a variety of materials such as glass or plastic. The container holds a DR5 antibody formulation that is effective for diagnosing or treating the disorder and may have a sterile access port (for example, the container may be an intravenous solution bag or a vial having a stopper pierceable by a hypodermic injection needle). The label on, or associated with, the container indicates that the formulation is used for diagnosing or treating the disorder of choice. The article of manufacture may further comprise a second container comprising water-for-injection, a pharmaceutically-acceptable solution, saline, Ringer's solution, or dextrose solution. It may further include other materials desirable from a commercial and user standpoint, including other buffers, diluents, filters, needles, syringes, and package inserts with instructions for use.

All patents, patent applications, publications, product descriptions, and protocols are cited throughout this application, the disclosures of which are incorporated herein by reference in their entireties. The section headings used herein are for organizational purposes only and are not to be construed as limiting the subject matter described.

### EXAMPLES

The following examples are offered for illustrative purposes only, and are not intended to limit the scope of the present invention in any way. Commercially available reagents referred to in the examples were used according to manufacturer's instructions unless otherwise indicated. The source of those cells identified in the following examples, and throughout the specification, by ATCC accession numbers is the American Type Culture Collection, Manassas, Va. In the next set of examples, common α-amino acids may be described by the standard one- or three-letter amino acid code when referring to intermediates and final products. By common α-amino acids is meant those amino acids incorporated into proteins under mRNA direction. Standard abbreviations are listed in The Merck Index, 10th Edition, pp Misc-2-Misc-3. Unless otherwise designated the common α-amino acids have the natural or “L”-configuration at the alpha carbon atom. If the code is preceded by a “D” this signifies the opposite enantiomer of the common α-amino acid. Modified or unusual α-amino acids such as norleucine (Nle) and ornithine (Orn) are designated as described in U.S. Patent and Trademark Office Official Gazette 1114 TMOG, May 15, 1990.

### Example 1

### Construction of scFv Library LSS-2331B

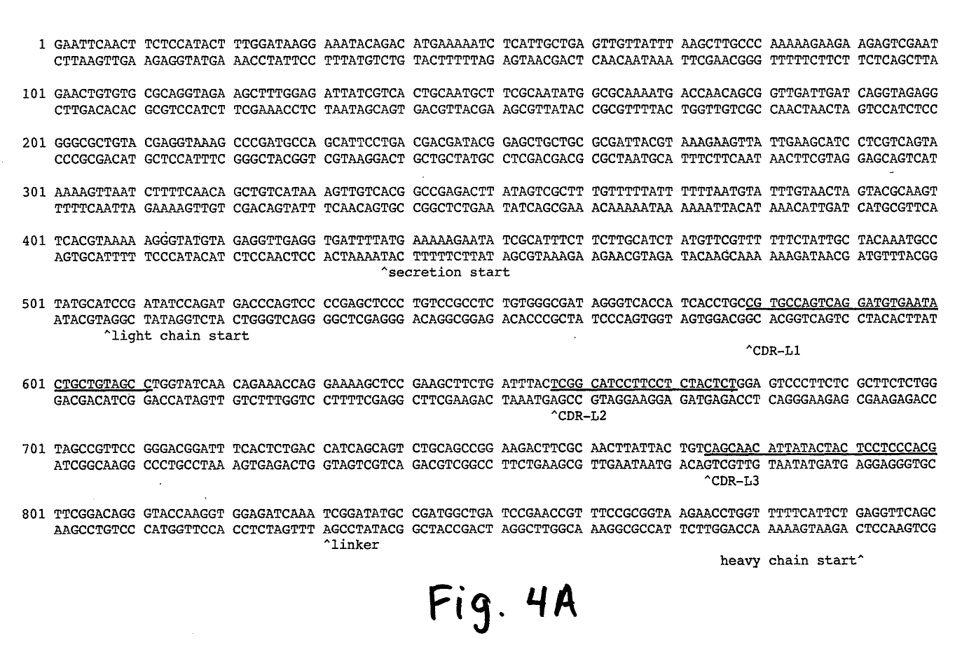

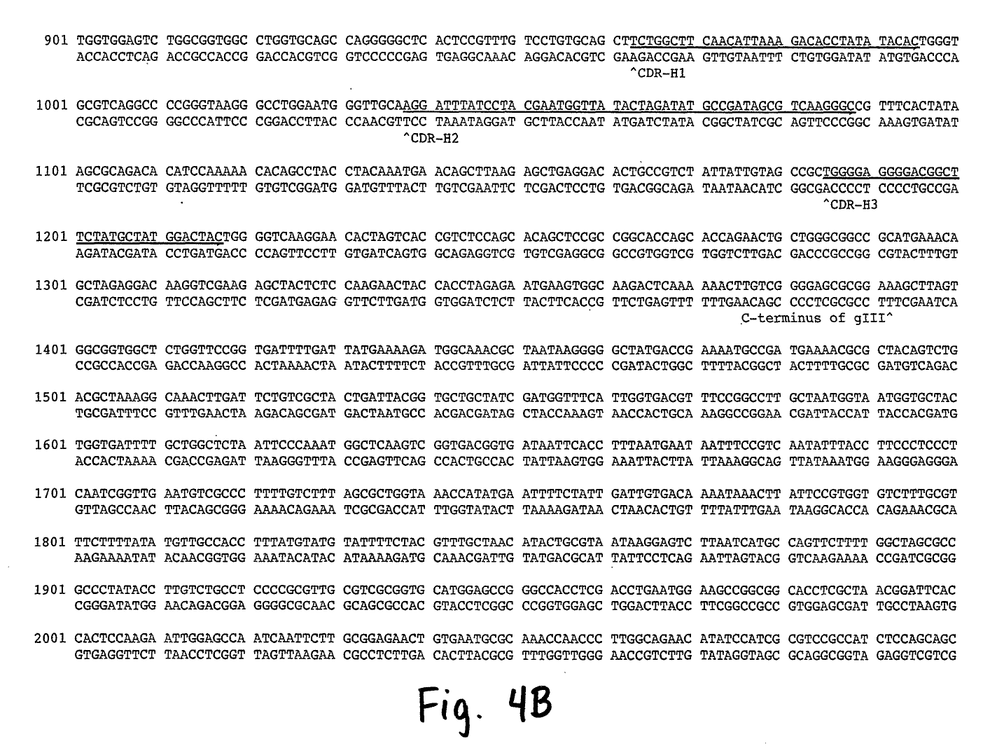

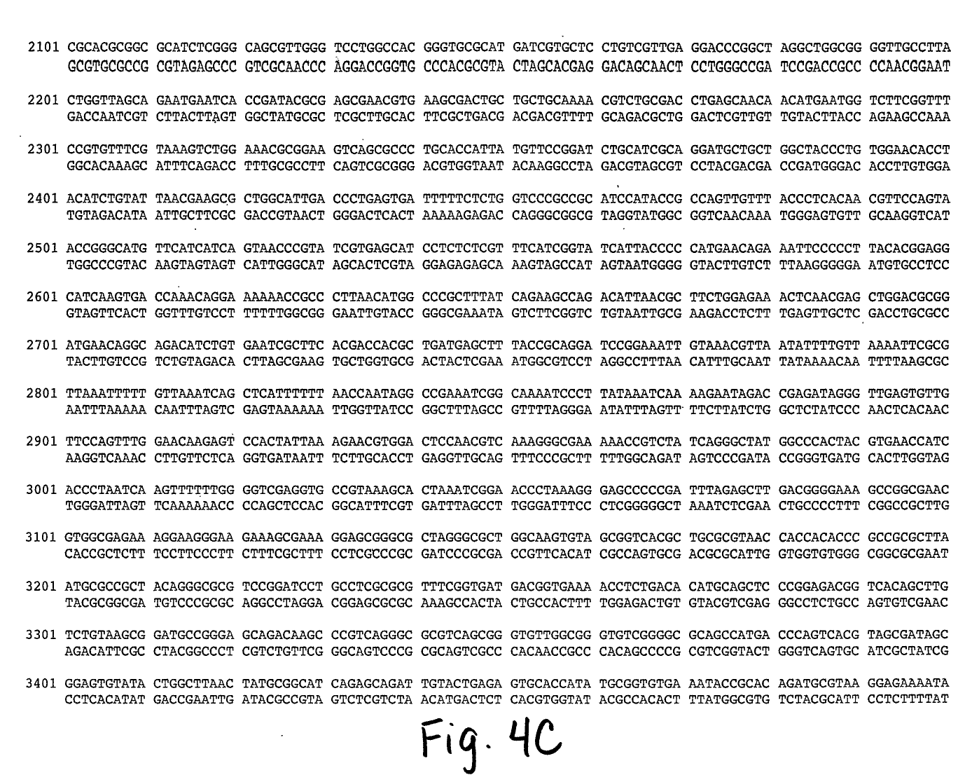

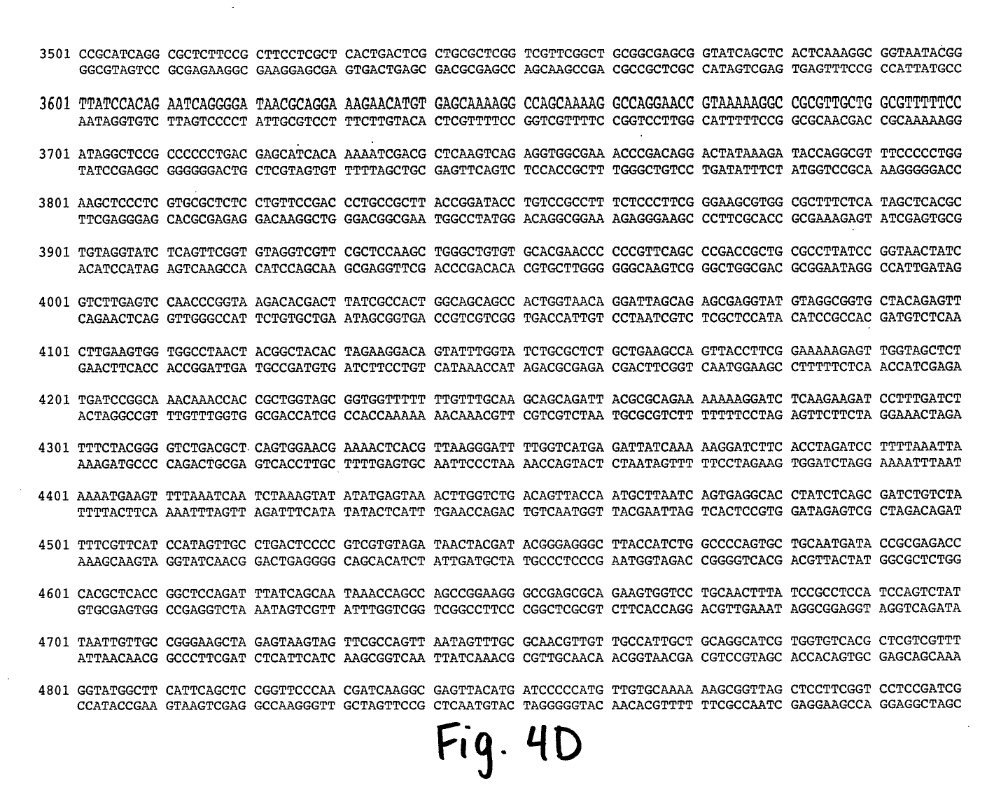

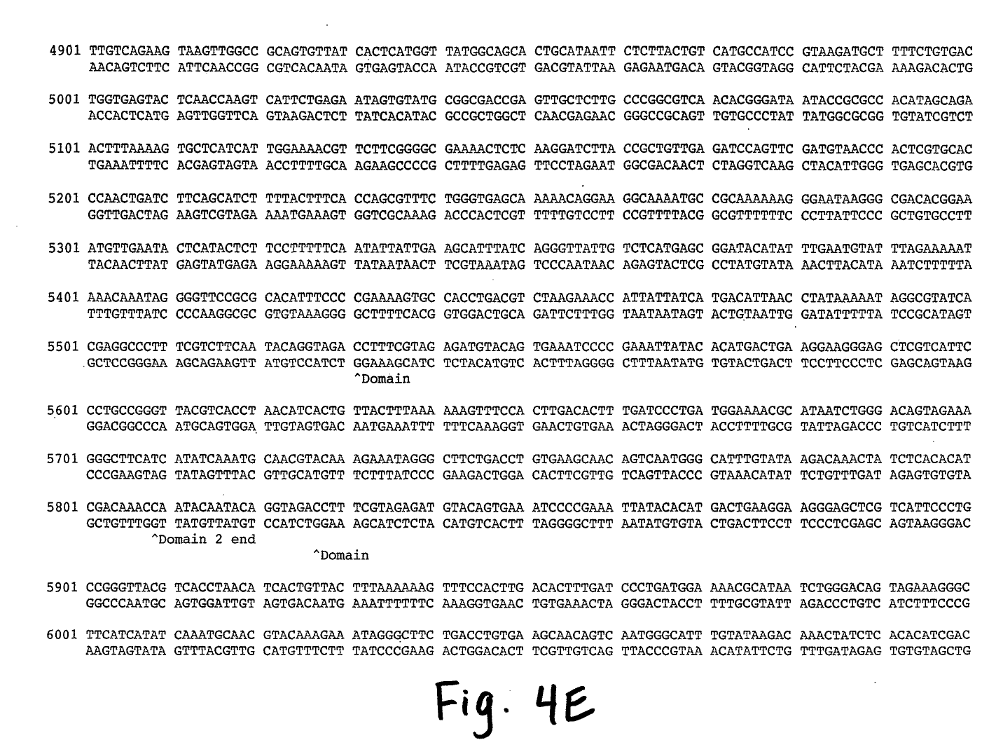

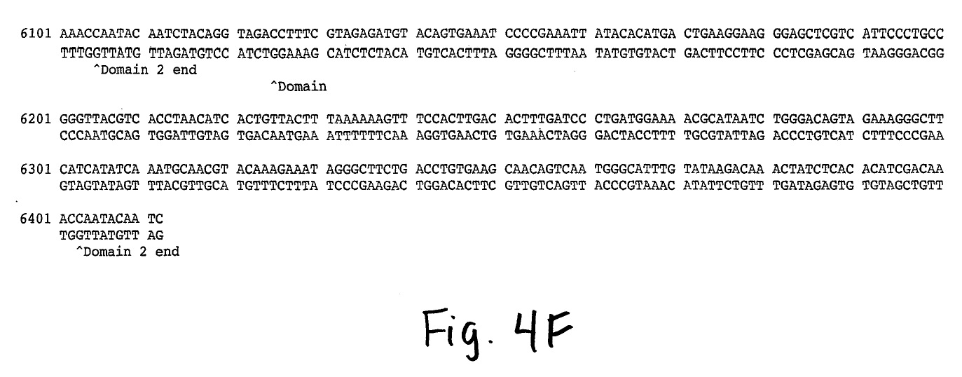

A phage-displayed scFv library, referred to as “LSS-2331B”, was constructed using a phagemid vector that resulted in the display of bivalent scFv moieties dimerized by a leucine zipper domain inserted between the scFv and the C-terminal domain of the gene-3 minor coat protein (P3C). This vector was designated “pS2072a” and comprises the sequence shown in FIG. 4. The vector comprises the humanized antibody 4D5 variable domains under the control of the alkaline phosphotase (phoA) promoter. The humanized antibody 4D5 is an antibody which has mostly human consensus sequence framework regions in the heavy and light chains, and CDR regions from a mouse monoclonal antibody specific for Her-2. The method of making the anti-Her-2 antibody and the identity of the variable domain sequences are provided in U.S. Pat. Nos. 5,821,337 and 6,054,297.

LSS-2331B was constructed with randomized residues in all three heavy chain CDRs. The specific residues that were randomized are follows: residues 28, 30, 31, 32, and 33 in CDR-H1; residues 50, 52, 53, 54, 56, and 58 in CDR-H2; residues 95, 96, 97, 98, 99, and 100 in CDR-H3. Additional diversity was introduced into CDR-H3 by replacing the 6 wild-type codons between positions 95 to 100 with varying numbers of degenerate codons (3 to 14).

Library LSS-2331B was constructed using the method of Kunkel et al., Methods Enzymol. (1987), 154:367-382) with previously described methods (Sidhu, S. S., et al., Methods Enzymol. (2000), 328:333-363). A unique “stop template” version of pS2072a (designated pS2072c) was constructed by substituting TAA stop codons in place of the codons at positions 30, 31, 32, 33, 53, 54, 56, 98, 99, 100, and 100a of the heavy chain. Mutagenic oligonucleotides with degenerate codons at the positions to be diversified were used to simultaneously introduce CDR diversity and repair the stop codons. The oligonucleotide sequences are shown below in Table 1.

TABLE 1

Mutagenic oligonucleotides used in the
construction of Libraries LSS-2331B.
Equimolar DNA degeneracies are represented
in the IUB code (W = A/T, R = A/G, V =
G/A/C, N = A/G/C/T, M = A/C, Y = C/T,
D = G/A/T, B = G/T/C, K = G/T, S = G/C,
H = A/T/C).

| | |
|---|---|
| Name | Sequence |

| | |
|---|---|
| H1-1 | TGT GGA GCT TCT GGC TTC WCC ATT RVN RVN WMY |
| | RNT ATA CAC TGG GTG CGT GAG |
| | (SEQ ID NO:116) |
| H2-1 | GGC CTG GAA TGG GTT GCA DBG ATT DHT CCA NMY |
| | DMT GGT DMT ACT DMT TAT GCC GAT AGC GTC AAG |
| | (SEQ ID NO:117) |
| H3-1 | GCC GTC TAT TAT TGT AGC CGC DVK DVK NNK TAC |
| | GCT ATG GAC TAC TGG GG |
| | (SEQ ID NO:118) |
| H3-2 | GCC GTC TAT TAT TGT AGC CGC DVK DVK DVK NNK |
| | TAC GCT ATG GAC TAC TGG GG |
| | (SEQ ID NO:119) |
| H3-3 | GCC GTC TAT TAT TGT AGC CGC DVK DVK DVK DVK |
| | NNK TAC GCT ATG GAC TAC TGG GG |
| | (SEQ ID NO:120) |
| H3-4 | GCC GTC TAT TAT TGT AGC CGC DVK DVK DVK DVK |
| | DVK NNK TAC GCT ATG GAC TAC TGG GG |
| | (SEQ ID NO:121) |
| H3-5 | GCC GTC TAT TAT TGT AGC CGC DVK DVK DVK DVK |
| | DVK DVK NNK TAC GCT ATG GAC TAC TGG GG |
| | ((SEQ ID NO:122) |
| H3-6 | GCC GTC TAT TAT TGT AGC CGC DVK DVK DVK DVK |
| | DVK DVK DVK NNK TAC GCT ATG GAC TAC TGG GG |
| | (SEQ ID NO:123) |
| H3-7 | GCC GTC TAT TAT TGT AGC CGC DVK DVK DVK DVK |
| | DVK DVK DVK DVK NNK TAC GCT ATG GAC TAC TGG |
| | GG |
| | (SEQ ID NO:124) |
| H3-8 | GCC GTC TAT TAT TGT AGC CGC DVK DVK DVK DVK |
| | DVK DVK DVK DVK DVK NNK TAC GCT ATG GAC TAC |
| | TGG GG |
| | (SEQ ID NO:125) |
| H3-9 | GCC GTC TAT TAT TGT AGC CGC DVK DVK DVK DVK |
| | DVK DVK DVK DVK DVK DVK NNK TAC GCT ATG GAC |
| | TAC TGG GG |
| | (SEQ ID NO:126) |
| H3-10 | GCC GTC TAT TAT TGT AGC CGC DVK DVK DVK DVK |
| | DVK DVK DVK DVK DVK DVK DVK NNK TAC GCT ATG |
| | GAC TAC TGG GG |
| | (SEQ ID NO:127) |
| H3-11 | GCC GTC TAT TAT TGT AGC CGC DVK DVK DVK DVK |
| | DVK DVK DVK DVK DVK DVK DVK DVK NNK TAC GCT |
| | ATG GAC TAC TGG GG |
| | (SEQ ID NO:128) |
| H3-12 | GCC GTC TAT TAT TGT AGC CGC DVK DVK DVK DVK |
| | DVK DVK DVK DVK DVK DVK DVK DVK DVK NNK TAC |
| | GCT ATG GAC TAC TGG GG |
| | (SEQ ID NO:129) |

Diversity was introduced into CDR-H1 and CDR-H2 with oligonucleotides H1-1 and H2-1, respectively. Diversity was introduced into CDR-H3 with an equimolar mixture of oligonucleotides H3-1, H3-2, H3-3, H3-4, H3-5, H3-6, H3-7, H3-8, H3-9, H3-10, H3-11, and H3-12. The mutagenic oligonucleotides for all CDRs to be randomized were incorporated into the pS2027c template simultaneously in a single mutagenesis reaction, so that simultaneous incorporation of all the mutagenic oligonucleotides resulted in the introduction of the designed diversity at each position and simultaneously repaired all the TAA stop codons, thus generating an open reading frame that encoded a scFv library member fused to a homodimerizing leucine zipper and P3C.

The mutagenesis reactions were electroporated into E. coli SS320 (Sidhu, S. S., et al., Methods Enzymol. (2000), 328:333-363), and the transformed cells were grown overnight in the presence of M13-KO7 helper phage (New England Biolabs, Beverly, Mass.) to produce phage particles that encapsulated the phagemid DNA and displayed Fab fragments on their surfaces. Library LSS-2331B contained 3×10¹⁰ unique members.

After thee library construction, sorting was conducted as described as follows using a 3 step sorting technique:

### Sort 1

1. Coat human DR5-ECD (see Table 9 below) on Maxisorp immunoplate 12 wells with 2 ug/ml, 100 ul/well, and incubate at 4° C. overnight.
2. Block the plate: Add 200 ul of PBS with casein for 1 hour at room temperature.
3. Block phage: Add blocking buffer (casein) to the phage solution at 1:1 ratio and incubate at room temperature for 1 hour.
4. Wash the plate 5 times with PT buffer (PBS+ 0.05% Tween 20).
5. Add 100 uL of library phage solution (from step 4) to the wells and incubate at room temperature for 2 hours with gentle shaking.
6. Wash the plate 10 times with PT buffer.
7. Elute with 50 ul of 0.1 M HCL, pH 2 at the first 12 wells for 20 minutes; Neutralize the eluant with 1.0M tris base (about ⅙ volume).
8. Infect 5 ml of X11-blue cells (OD600=1.0) with 1.5 ml phage eluant. Grow 20 minutes at 37° C. Titre on LB/carb plates and transfer the culture to 50 mL of 2YT/carbVCS and grow overnight at 37° C.
9. Spin down the cells and save the supernatant which is ready for the next sort.

### Sort 2

1. Coat 12 wells with human DR5-ECD.
2. Block the plate: Add 200 ul super block (Pierce Chemicals, Product # 37515) for 1 hour at room temperature.
3. Block the phage: Add casein to phage supernatant (from sort 1, step 12) at ratio 1:1 and incubate at room temperature for 1 hour.
4. Infect 1 ml of X11-blue cells (OD600=1.0) with 0.3 ml phage eluant. Grow 20 minutes at 37° C. Titre on LB/carb plates and transfer the culture to 25 mL of 2YT/carbVCS and grow overnight at 37° C.
5. Spin down the cells and save the supernatant which is ready for the next sort.

### Sort 3

1. Coat 12 wells with human DR5-ECD.
2. Block the plate: Add 200 ul PBS/BSA for 1 hour at room temperature.
3. Block the phage: Add super block to phage supernatant at ratio 1:1 and incubate at room temperature for 1 hour.
4-5. The same as above.

### Example 2

### Construction of scFv Library LSS-2344F

A library referred to as “LSS-2344F” was constructed as described for LSS-2331B in Example 1, except for the following differences.

The stop template (pG4503f) differed from pS2072c in one codon that resulted in a point mutation in the heavy chain (H91S). The sequences of the mutagenic oligonucleotides used for library construction are shown below in Table 2.

TABLE 2

Mutagenic oligonucleotides used in the
construction of Libraries LSS-2344F.
Equimolar DNA degeneracies are represented
in the IUB code (W = A/T, R = A/G, V =
G/A/C, N = A/G/C/T, M = A/C, Y = C/T,
D = G/A/T, B = G/T/C, K = G/T, S = G/C,
H = A/T/C).

| | |
|---|---|
| Name | Sequence |

| | |
|---|---|
| H1-2 | GCA GCT TCT GGG TTC ACC ATT AVT RRT WMY KMT |
| | ATA CAC TGG GTG CGT CAG |
| | (SEQ ID NO:130) |
| H1-3 | GCA GCT TCT GGG TTC ACC ATT AVT RRT WMY KGG |
| | ATA CAC TGG GTG CGT CAG |
| | (SEQ ID NO:131) |
| H1-4 | GCA GCT TCT GGC TTC ACC ATT AVT RVM WMY KMT |
| | ATA CAC TGG GTG CGT CAG |
| | (SEQ ID NO:132) |
| H1-5 | GCA GCT TCT GGC TTC ACC ATT AVT RVM WMY KGG |
| | ATA CAC TGG GTG CGT CAG |
| | (SEQ ID NO:133) |
| H2-2 | AAG GGC CTG GAA TGG GTT GST DHT ATT WMT CCT |
| | DMT RRC GGT DMT ACT DAC TAT GCC GAT AGC GTC |
| | AAG GG |
| | (SEQ ID NO:134) |
| H2-3 | AAG GGC CTG GAA TGG GTT GST DGG ATT WMT CCT |
| | DMT RRC GGT DMT ACT DAC TAT GCC GAT AGC GTC |
| | AAG GGC |
| | (SEQ ID NO:135) |
| H2-4 | AAG GGC CTG GAA TGG GTT GST DHT ATT DMT CCT |
| | NMT RRC GGC DMT ACT DAC TAT GCC GAT AGC GTC |
| | AAG GGC |
| | (SEQ ID NO:136) |
| H2-5 | AAG GGC CTG GAA TGG GTT GST DGG ATT DMT CCT |
| | NMT RRC GGC DMT ACT DAC TAT GCC GAT AGC GTC |
| | AAG GGG |
| | (SEQ ID NO:137) |
| H3-13 | ACT GCC GTC TAT TAT TGT GCT CGT NNS NNS NNS |
| | NNS TAC GBT ATG GAC TAC TGG GGT CAA |
| | (SEQ ID NO:138) |
| H3-14 | ACT GCC GTC TAT TAT TGT GCT CGT NNS NNS NNS |
| | NNS KSG GBT ATG GAC TAC TGG GGT CAA |
| | (SEQ ID NO:139) |
| H3-15 | ACT GCC GTC TAT TAT TGT GCT CGT NNS NNS NNS |
| | NNS NNS TAC GBT ATG GAC TAC TGG GGT CAA |
| | (SEQ ID NO:140) |
| H3-16 | ACT GCC GTC TAT TAT TGT GCT CGT NNS NNS NNS |
| | NNS NNS KSG GBT ATG GAC TAC TGG GGT CAA |
| | (SEQ ID NO:141) |
| H3-17 | ACT GCC GTC TAT TAT TGT GCA ARA DVK DVK DVK |
| | DVK DVK NNK TAC GCT ATG GAC TAC TGG GGT CAA |
| | (SEQ ID NO:142) |
| H3-18 | ACT GCC GTC TAT TAT TGT GCA ARA TGG NVT DVK |
| | DVK DVK DVK DSG GCT ATG GAC TAC TGG GGT CAA |
| | (SEQ ID NO:143) |
| H3-19 | ACT GCC GTC TAT TAT TGT GCA ARA DVK DVK DVK |
| | DVK DVK DVK KSG GCT ATG GAC TAC TGG GGT CAA |
| | (SEQ ID NO:144) |
| H3-20 | ACT GCC GTC TAT TAT TGT GCA CGT DVK DVK DVK |
| | DVK DVK DVK DVK TAC GCT ATG GAC TAC TGG GGT |
| | CAA |
| | (SEQ ID NO:145) |
| H3-21 | ACT GCC GTC TAT TAT TGT GCA CGT DVK DVK DVK |
| | DVK DVK DVK DVK DSG GCT ATG GAC TAC TGG GGT |
| | CAA |
| | (SEQ ID NO:146) |
| H3-22 | ACT GCC GTC TAT TAT TGT GCA CGT DVK DVK DVK |
| | DVK DVK DVK DVK DVK TAC GCT ATG GAC TAC TGG |
| | GGT CAA |
| | (SEQ ID NO:147) |
| H3-23 | ACT GCC GTC TAT TAT TGT GCA CGT DVK DVK DVK |
| | DVK DVK DVK DVK DVK DSG GCT ATG GAC TAC TGG |
| | GGT CAA |
| | (SEQ ID NO:148) |

Diversity was introduced into CDR-H1 with oligonucleotides H1-2, H1-3, H1-4, and H1-5 (2:1:2:1 ratio) Diversity was introduced into CDR-H2 with oligonucleotides H2-2, H2-3, H3-4, and H4-5 (2:1:2:1 ratio). Diversity was introduced into CDR-H3 with an equimolar mixture of oligonucleotides H3-13, H3-14, H3-15, H3-16, H3-17, H3-18, H3-19, H3-20, H3-21, H3-22, and H3-23. Library LSS-2344F contained 1.5×10¹⁰ unique members.

Sorting was then conducted using the 3 step sort methods described in Example 1.

### Example 3

### Construction of Fab Library LSS-2369B

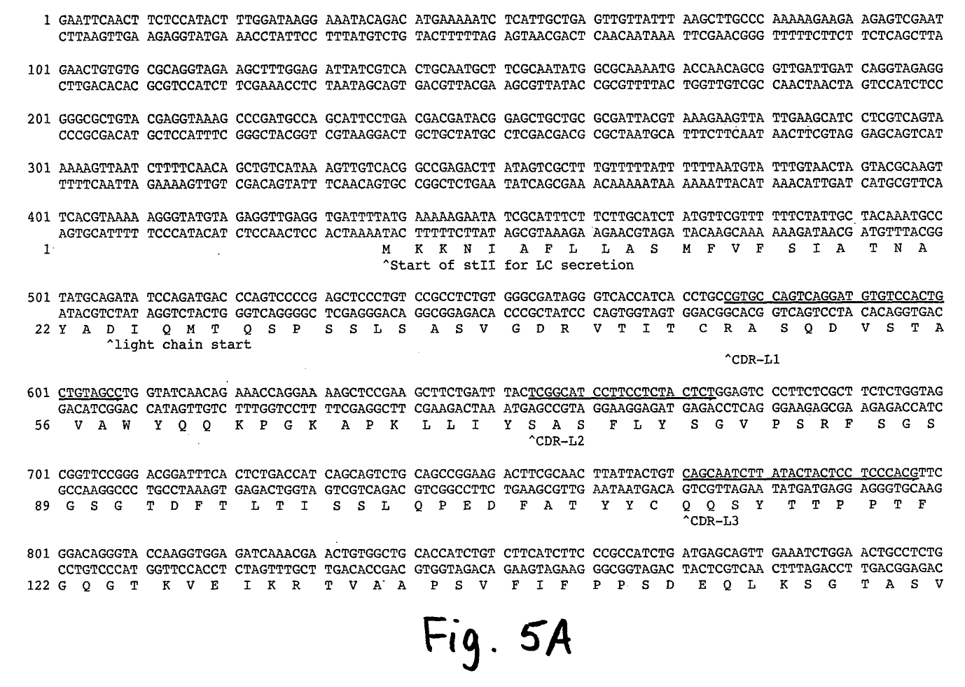

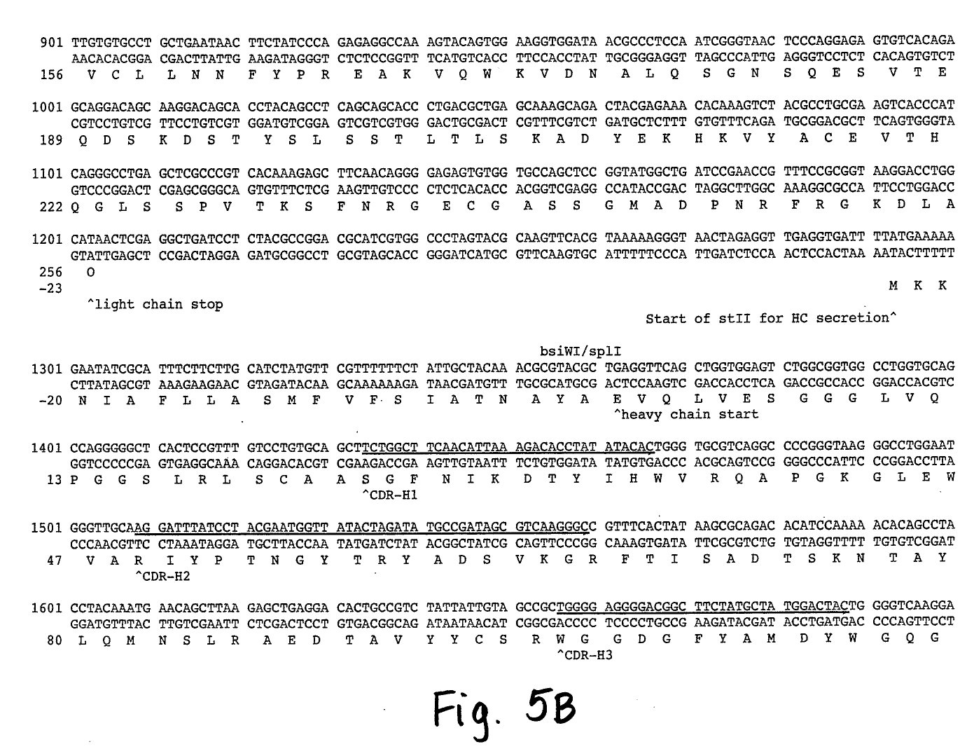

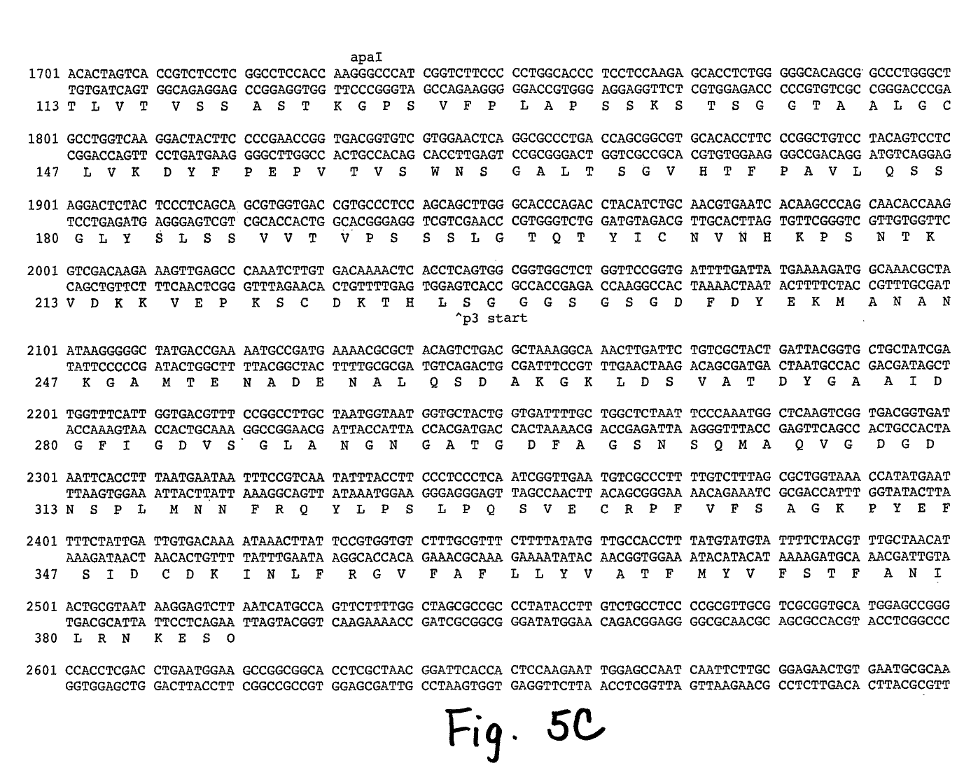

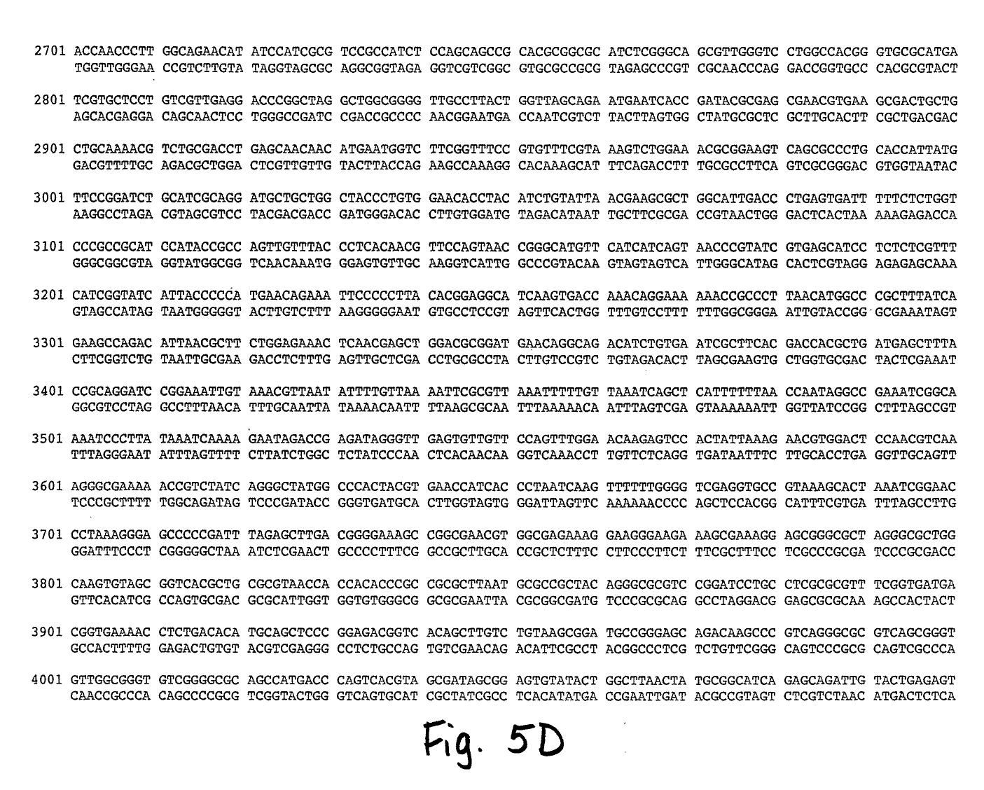

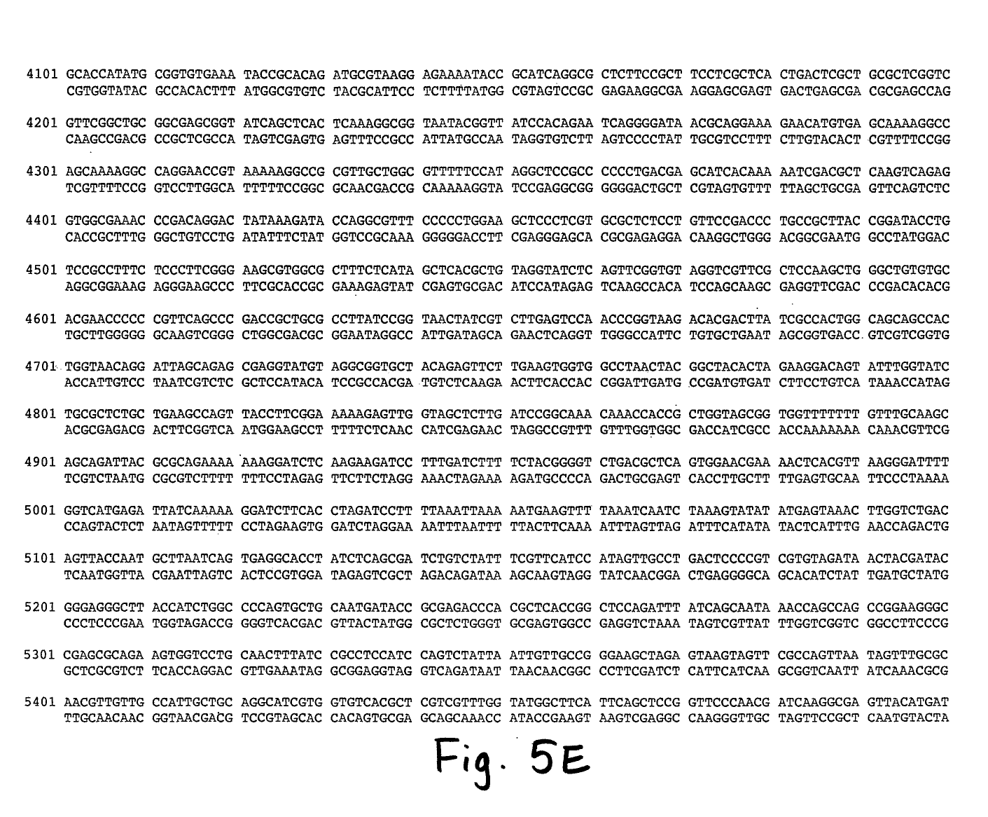

Phage-displayed Fab library, “LSS-2369B”, was constructed using a phagemid vector that resulted in the display of Fab moieties fused to the C-terminal domain of the gene-3 minor coat protein (P3C). This vector was designated pV-0350-2 and comprises the sequence shown in FIG. 5. The vector comprises the humanized antibody 4D5 Fab with 3 mutations in the light chain (N30S, R66G, and H91S), under the control of the alkaline phosphotase (phoA) promoter. The humanized antibody 4D5 is an antibody which has mostly human consensus sequence framework regions in the heavy and light chains, and CDR regions from a mouse monoclonal antibody specific for Her-2. The method of making the anti-Her-2 antibody and the identity of the variable domain sequences are provided in U.S. Pat. Nos. 5,821,337 and 6,054,297.

LSS-2369B was constructed with randomized residues in all three heavy chain CDRs. The specific residues that were randomized are follows: residues 28, 30, 31, 32, and 33 in CDR-HL; residues 50, 52, 53, 54, 56, and 58 in CDR-H2; residues 95, 96, 97, 98, 99, 100, 100a, 100b, and 100c in CDR-H3. Additional diversity was introduced into CDR-H3 by replacing the 9 wild-type codons between positions 95 to 100 with varying numbers of degenerate codons (7, 8, 9, 10, or 12).

Library LSS-2369B was constructed using the method of Kunkel et al., Methods Enzymol. (1987), 154:367-382) with previously described methods (Sidhu, S. S., et al., Methods Enzymol. (2000), 328:333-363). A unique “stop template” version of pV-o350-2 (designated pV-0350-2b) was constructed by substituting TAA step codons in place of the codons at positions 30, 31, 32, 33, 53, 54, 56, 98, 99, 100, and 100a of the heavy chain. Mutagenic oligonucleotides with degenerate codons at the positions to be diversified were used to simultaneously introduce CDR diversity and repair the stop codons. Diversity as introduced into CDR-H1 and CDR-H2 with oligonucleotides H1-1 and H2-1, respectively (shown in Table 1 above). Diversity was introduced into CDR-H3 with an equimolar mixture of oligonucleotides H3-24, H3-25, H3-26, H-3-2, and H3-28 (shown below in Table 3).

TABLE 3

Mutagenic oligonucleotides used in the
construction of Libraries LSS-2369B.
Equimolar DNA degeneracies are represented
in the IUB code (W = A/T, R = A/G, V =
G/A/C, N = A/G/C/T, M = A/C, Y = C/T,
D = G/A/T, B = G/T/C, K = G/T, S = G/C,
H = A/T/C).

| | |
|---|---|
| Name | Sequence |

| | |
|---|---|
| H3-24 | GCC GTC TAT TAT TGT GCT CGC NNK NNK NNK NNK |
| | NNK WTK GAC TAC TGG GGT CAA |
| | (SEQ ID NO:149) |
| H3-25 | GCC GTC TAT TAT TGT GCT CGC NNK NNK NNK NNK |
| | NNK NNK WTK GAC TAC TGG GGT CAA |
| | (SEQ ID NO:150) |
| H3-26 | GCC GTC TAT TAT TGT GCT CGC NNK NNK NNK NNK |
| | NNK NNK NNK WTK GAC TAC TGG GGT CAA |
| | (SEQ ID NO:151) |
| H3-27 | GCC GTC TAT TAT TGT GCT CGC NNK NNK NNK NNK |
| | NNK NNK NNK NNK WTK GAC TAC TGG GGT CAA |
| | (SEQ ID NO:152) |
| H3-28 | GCC GTC TAT TAT TGT GCT CGC NNK NNK NNK NNK |
| | NNK NNK NNK NNK NNK WTK GAC TAC TGG GGT CAA |
| | (SEQ ID NO:153) |

The mutagenic oligonucleotides for all CDRs to be randomized were incorporated into the pV-0350-2b template simultaneously in a single mutagenesis reaction, so that simultaneous incorporation of all the mutagenic oligonucleotides resulted in the introduction of the designed diversity at each position and simultaneously repaired all the TAA stop codons, thus generating an open reading frame that encoded a Fab library member fused to P3C.

The mutagenesis reactions were electroporated into E. coli SS320 (Sidhu, S. S., et al., Methods Enzymol. (2000), 328:333-363), and the transformed cells were grown overnight in the presence of M13-KO7 helper phage (New England Biolabs, Beverly, Mass.) to produce phage particles that encapsulated the phagemid DNA and displayed Fab fragments on their surfaces. Library LSS-23696B contained 6.2×10¹⁰ unique members.

Sorting was then conducted using the 3 step sort methods described in Example 1.

### Example 4

### Selection of Specific Antibodies from the Phage Libraries

Phage from each library described above (Examples 1, 2, and 3) were cycled separately through rounds of binding selection to enrich for clones binding to human DR5-ECD (see Table 9 below). The binding selections were conducted using previously described methods (Sidhu et al., supra).

NUNC 96-well Maxisorp immunoplates were coated overnight at 4° C. with capture target (hDR5-ECD at 5 ug/mL in PBS) and blocked for 2 hours with bovine serum albumin (BSA) (Sigma). After overnight growth at 37° C., phage were concentrated by precipitation with PEG/NaCl and resuspended in PBS, 0.5% BSA, 0.1% Tween 20 (Sigma), as described previously (Sidhu et al., supra). Phage solutions (10¹² phage/mL) were added to the coated immunoplates. Following a 2 hour incubation to allow for phage binding, the plates were washed 10 times with PBS, 0.05% Tween 20. Bound phage were eluted with 0.1 M HCl for 10 minutes, and the eluant was neutralized with 1.0 M Tris base. Eluted phage were amplified in E. coli XL1-blue and used for further rounds of selection. The libraries LSS-2344F and LSS-2331B were subjected to 3 or 4 rounds of selection for binding to hDR5-ECD, respectively. Library LSS-2369B was subjected to 2 rounds of selection against hDR5-ECD, followed by a round of selection (round 2a) against an anti-gD epitope antibody to enrich for clones displaying Fab (there is a gD epitope fused to the C-terminus of the light chain), followed a third round of selection against hDR5-ECD.

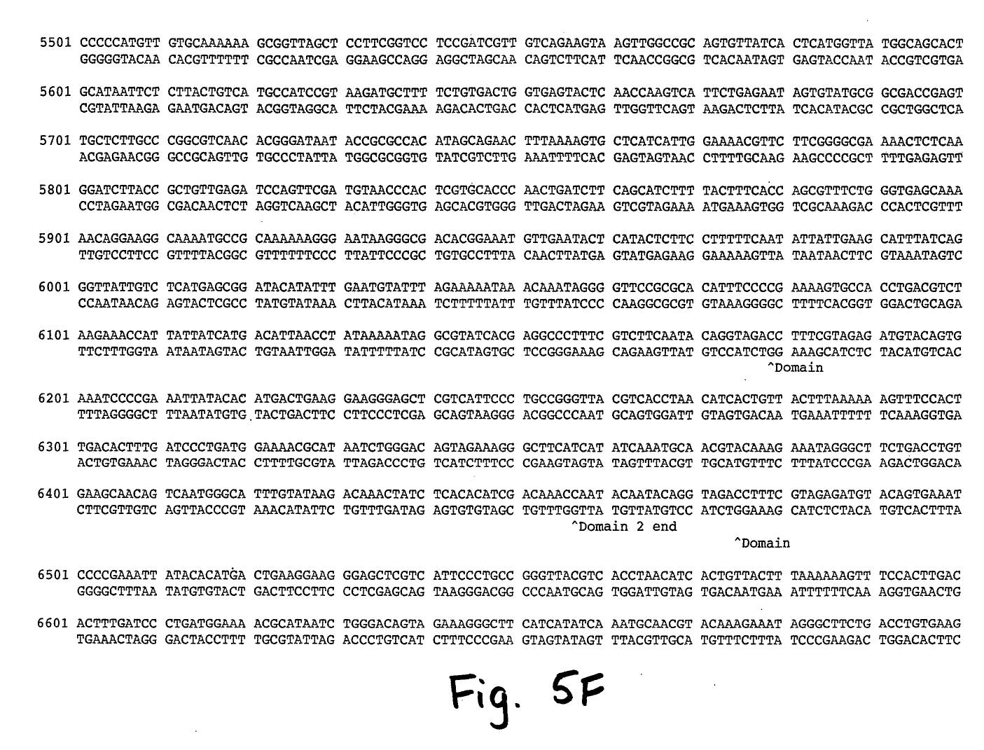

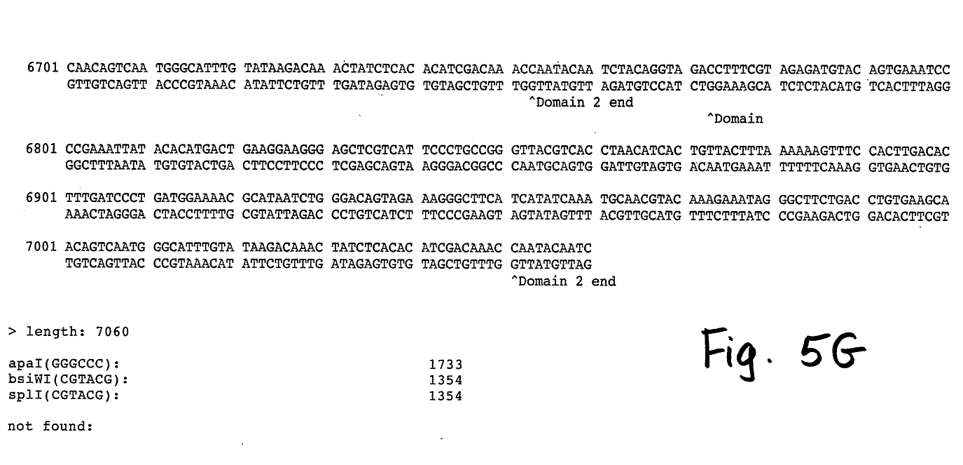

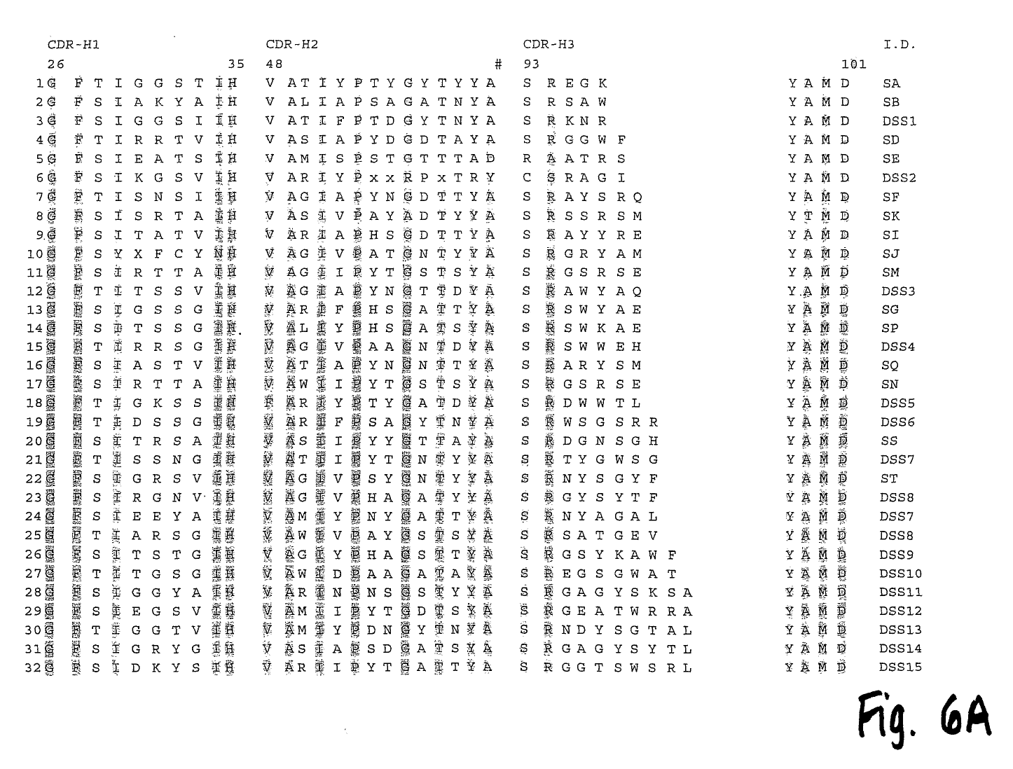

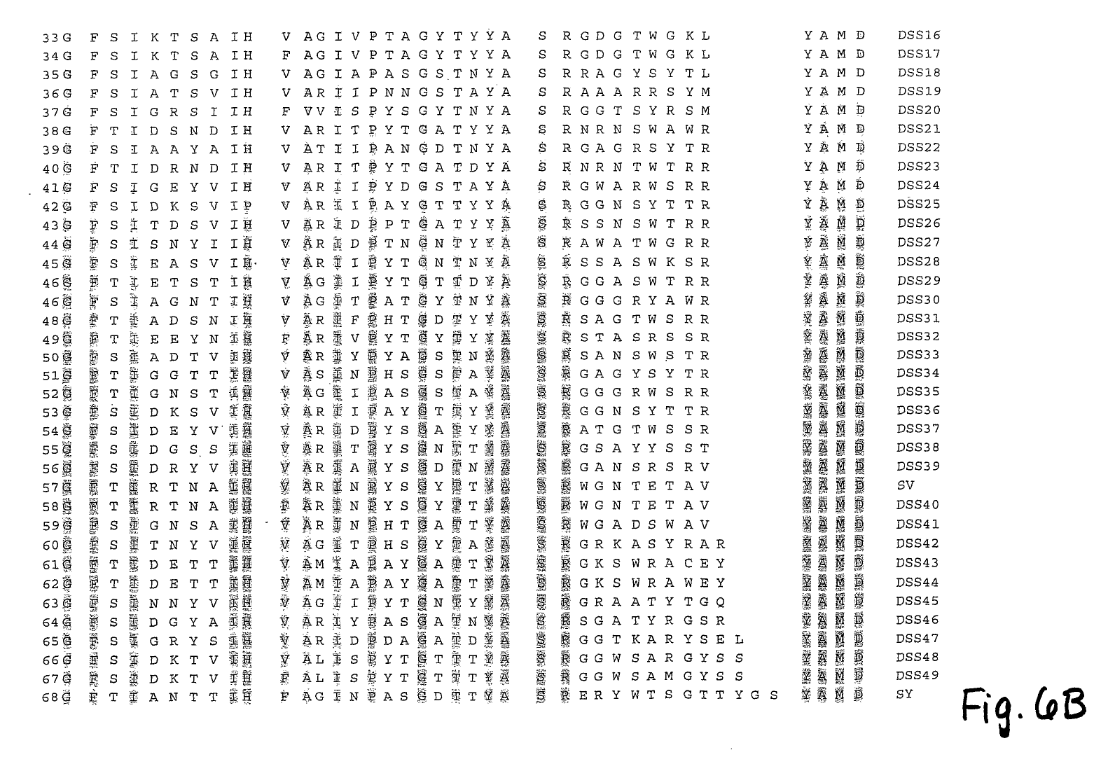

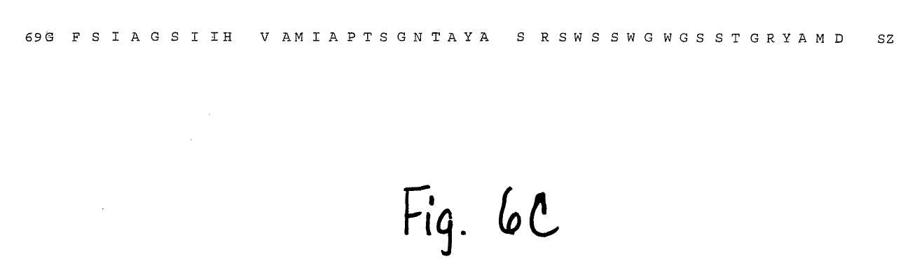

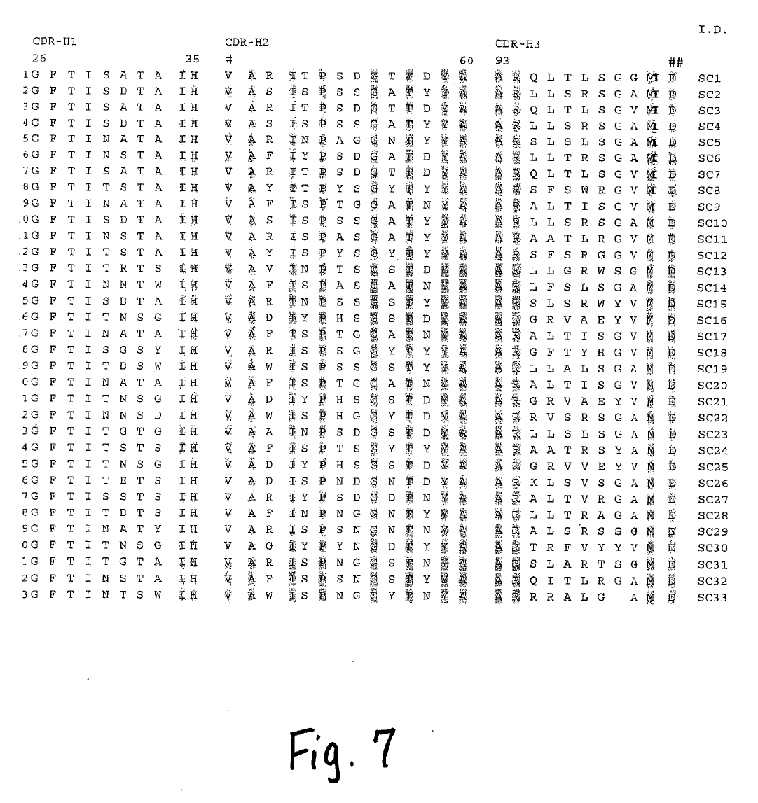

For each library, individual clones from the final round of selection were grown in a 96-well format in 500 uL of 2YT broth supplemented with carbenicillin and M13-KO7, and the culture supernatants were used directly in phage ELISAs (Sidhu et al., supra) to detect phage-displayed antibodies that bound to plates coated with hDR5-ECD but not to plates coated to BSA. Positive binding clones were defined as those that exhibited ELISA signals on plates coated with hDR5-ECD that were at least 10-fold higher than signals on plates coated with BSA (controls). ELISA assay testing was also conducted to confirm that the positive binding clones were specific for DR5 receptor and did not exhibit cross-reactivity with DR4, DcR1 or DcR2 receptors (e.g., the other receptors to which Apo-2 ligand binds) (data not shown). Positive binding clones from each library were subjected to DNA sequencing analysis, using standard methods. For LSS-2331B, 180 clones were sequenced to reveal 65 unique sequences (FIG. 6). For LSS-2344F, 176 clones were sequenced to reveal 33 unique sequences (FIG. 7). For LSS-2369B 96 clones were sequenced to reveal 3 unique sequences (FIG. 8).

The results of the phage ELISA of clones selected from the library LSS-2331B (see Example 1) are shown in Table 4 below. The “Identifier” in Table 4 refers to the name or code assigned to the particular cloned antibody and the respective Identifiers correspond to those included in FIG. 6. The binding of each of these antibodies to human DR5-ECD and to cynomolgous (“cyno”) DR5-IgG (see Table 9 below) for comparison is shown in Table 4.

TABLE 4
Phage ScFv Elisa

| | | Cyno DR5- |
|---|---|---|
| | Human DR5-ECD | IgG |
| Identifier | (nM) | (nM) |
| SB | 63 | >500 |
| SD | 200 | >500 |
| SE | 100 | >500 |
| SG | 200 | >500 |
| SI | 125 | >500 |
| SP | 39 | >500 |
| SJ | 50 | >500 |
| SK | 80 | 79.4 |
| ST | 100 | 100 |
| SS | 100 | >500 |
| SV | 25 | 20 |
| SY | 50 | 105 |
| SZ | 50 | >500 |

The results of the phage ELISA of clones selected from the Fab library (see Example 3) are shown in the Table 5 below. The “Identifier” in Table 5 refers to the name or code assigned to the particular cloned antibody and the respective Identifiers correspond to those included in FIG. 8. The binding of each of these antibodies to human DR5-ECD (“HDR5-ECD”), human DR5-IgG (“HDR5-IgG”, Table 9), murine DR5-IgG (“MDR5-IgG”, Table 9), and to cynomolgous DR5-IgG (“CDR5-IgG”, Table 9) for comparison is shown in Table 5. “N.D.” refers to “not determined”.

TABLE 5

| | | | | | | | | | | | HDR5- | HDR5- | MDR5- | CDR5 |
|---|---|---|---|---|---|---|---|---|---|---|---|---|---|---|
| | | | | | | | | | | | ECD | IgG | IgG | IgG |
| | | | | | | | | | | | Ic50 | Ic50 | Ic50 | Ic50 |
| Identifier | | 95 | 96 | 98 | | | | | | | (nM) | (nM) | (nM) | (nM) |
| BdF1 | Fab | R | L | A | L | V | R | M | W | M | 2 | 3 | N.D. | 2 |
| Bd001 | Fab | N | V | R | R | R | K | P | T | F | 57 | 50 | N.D. | 79 |
| Bd002 | Fab | N | V | R | M | R | K | P | T | L | 42 | 22 | N.D. | 35 |

### Example 5

### Preparation of Fab Proteins Using E. coli Expression

Colonies (in 34B8) were picked in 5 ml 2YT +50 ug/ml carb, and the cells were grown to 1.5-2.5 OD at 37° C. 5 ml of culture was inoculated to 500 ml complete C.R.A.P. media +50 ug/ml carb, and then grown 18-24 hours at 30° C. The cells were spun down and the supernatant was decanted. The pellet was frozen at −20 C overnight.

The cell pellet was thawed on ice and the following was added: a) 20 ml TE (5 ml/g); 20 ul PMSH (5 ul/g); 4 ul 1M Benzamidine(1 ul/g); 2 ml 250 mM EDTA (0.4 ml/g). The cells were re-suspended completely and placed on ice for at least 1 hour. The shocked cells were spun down at 15 Krpm for 60 minutes. The supernatant was purified or the cells or homogenized (ultraturex) for 5 minutes. The cells were broken down with a microfluidizer, and spun down at 15K for 60 minutes. The supernatant was filtered through a 0.45 um filter, and loaded on a protein column (Pre wash the column with TE buffer). The column was washed with TE buffer, and then eluted with 0.1M acetic acid +1 mM EDTA, neutralized with 1M Tris, pH 8, and subsequently exchanged into PBS buffer. Measurements at OD 280 (conc=10D/0.4=mh/ml) were then made.

The expressed proteins were then tested in the following ELISA. Microtiter plate wells were coated with 80 ul of lug/ml human DR5-ECD (Table 9) (in 50 mM sodium carbonate pH 9.6) at 4° C. overnight. The coat was removed, blocked with 200 ul PBS/0.1% BSA/0.05% tween 20, and incubated 1 hour at room temperature. Competing receptor solutions (100 ul/sample) were prepared and appropriate subsaturating Biotin ladeled antibody (predetermined from antibodies dilution series) were prepared with DR5 receptor at different concentrations. The mixtures were incubated at room temperature for 2 hours. The plates which has been coated with DR5-ECD were shaken and rinsed 10 times with PBS+0.05% tween 20. 80 ul of the mixture of competing receptor and biotin labeled antibodies was transferred from the non-sticky plate to DR5 coated plate and incubated 20 minutes at room temperature. A 1:5000 dilution was made of HRP conjugate streptavidin (Zymed) into binding buffer. The plates were rinsed 10 times with PBS/tween 20, and 80 ul of HRP-streptavidin diluent was added and incubated 1 hour at room temperature. TMB peroxidase substrate and peroxidase solution B were mixed at equal volume. The plates were rinsed 10 times with PBS/tween 20, 80 ul of substrate was added, and then incubated as required to develop and stop with 80 ul 2.5M H₂S0₄.

The results of this ELISA testing antibody “BdF2” (see antibody I.D. in FIG. 8) are shown in Table 6 below. The binding of antibody BdF2 to human DR5-ECD, human DR5-IgG (see Table 9), human DR4-IgG (see Table 9) murine DR5-IgG (Table 9), and to cynomolgous DR5-IgG (“Cyno DR5-IgG”, Table 9) for comparison is shown in Table 6. “N.D.” refers to “not determined”.

TABLE 6
Fab abs

| | Human | Human | Human | Cyno | Murine | Murine |
|---|---|---|---|---|---|---|
| | DR5- | DR5 | DR4 | DR5 | DR5 | DR5 |
| Identifier | ECD | IgG | IgG | IgG | ECD | IgG |
| (I.D.) | (nM) | (nM) | (nM) | (nM) | (nM) | (nM) |
| BdF2 | 6.6 | / | N.D. | 1995 | N.D. | N.D. |

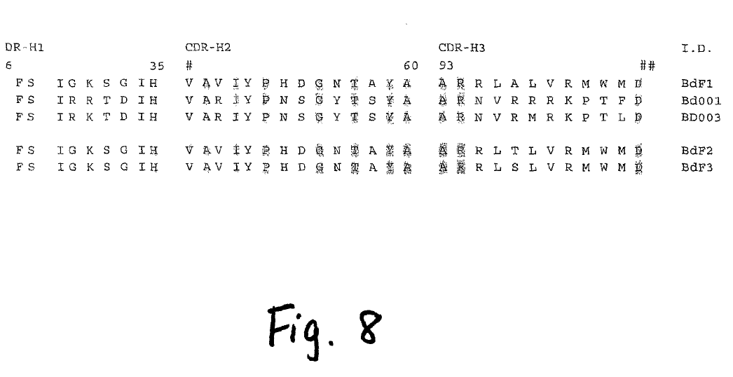

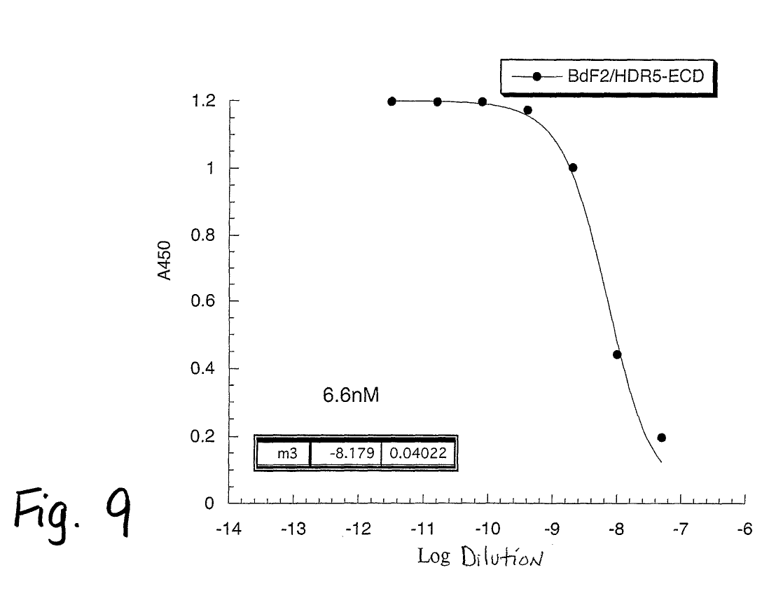

The results of the assay showing binding of Bdf2 to human DR5-ECD is also illustrated in FIG. 9.

### Example 6

### Binding Assays

Binding assays were conducted using BIAcore analyses. CM5 clips (Biocare) were warmed up to room temperature for at least a half hour. The BIAcore instrument was opened and the chips were docked into the instrument. Priming was conducted with running buffer (PBS/0.05%Tween-20/0.01% NaAzide) and then normalized with 70% Glycerol. A sensogram was run according to manufacturer instructions, immobilize protein solute ons (acetate buffer ph 5.5) were prepared and proteins were diluted at 20 ug/ml. The chips were activated with ECD and NHS. 5-30 ul proteins (Human DR5-ECD, Cyno DR5-IgG, murine DR5-ECD, or human DR4-IgG, all of which are described in Table 9) were injected at 20 ul/mins until the proteins were immobilized at 100 RU. The chips were then blocked with 1M ethanolamine. The samples were started at concentrations of 50 nM-500 nM, then 1:1 dilutions.

The data were analyzed using the Biaevaluation software program to measure protein kinetics.

The results of the Biacore assay for ScFv antibodies selected from the library described in Example 1 are reported in Table 7 below:

TABLE 7

| | Human | Cyno |
|---|---|---|
| | DR5 | DR5 |
| | ECD | IgG |
| Identifier | nM | nM |
| SB | 304 | |
| SD | 149 | |
| SE | 472 | |
| SJ | 18000 | |
| SK | 24 | 13 |
| SP | 86 | |
| SS | 167 | |
| ST | 18 | 2 |
| SV | 2 | 2 |
| SY | 554 | 92000 |
| SZ | 1575 | |

The results of the Biacore assay for selected Fab antibodies from the library described in Example 2 are reported in Table 8 below:

TABLE 8

| | Human | Human | Cyno | Murine |
|---|---|---|---|---|
| | DR5 | DR4 | DR5 | DR5 |
| | ECD | IgG | ECD | ECD |
| Identifier | (nM) | (nM) | (nM) | (nM) |
| Abs | | | | |
| Fc | | | | |
| BdF2* | 0.6 | N.D | 31 | N.D |
| BdF2** | 0.71 | N.D | 27 | N.D |
| Abs | | | | |
| Fab | | | | |
| BdF1 | 4.8 | N.D. | 12.3 | N.D |
| BdF2 | 11 | N.D. | 5.5 | N.D |
| BF3 | 8.5 | N.D | 3.0 | N.D |

*Protein purified from CHO cells
**Protein purified from E. coli
N.D. - refers to “not determined”

X-ray crystal structure studies of the Fab antibody called “BdF1” has revealed that in its binding to the human DR5 receptor, the CDR-H3 region of the antibody makes extensive contacts with a region of the DR5 receptor that overlaps with the Apo2L/TRAIL binding site, and that the residues in that CDR-H3 region are buried in the interface. (The crystal structure of the complex formed between Apo-2L/TRAIL and DR5 is described in Hymowitz et al., Molecular Cell, 4:563-571 (1999); see also WO 01/19861 published Mar. 22, 2001). Although not fully understood, it is believed that such may represent a potential hot-spot for binding on the DR5 receptor surface, which is exploited by the Apo-2 ligand/TRAIL and the Fab antibody identified in the phage-display techniques described in Example 3.

### Example 7

### In vitro Biological Assay of Selected Antibodies

Two fold serial dilutions of control standard and antibody “BdF2” (see FIG. 8) were performed in 96-well tissue culture plates (Falcon). Apo-2 ligand (amino acids 114-281, described in PCT US00/17579) was tested for comparison. Colo-205 (20000 cells/well) human colon carcinoma cells (ATCC) were seeded into the 96-well plates. The plates were incubated at 37° C. for 24 hours. AlamarBlue (Trek Diagnostic Systems, Inc.) was added to the wells for the last 3 hours of the 24 hours incubation time. Fluorescence was read using a 96-well fluorometer with excitation at 530 nm and emission of 590 nm. The results are expressed in relative fluorescence units (RFU). For data analysis the 4-parameter curve fitting program (Kaleidagraph) was used.

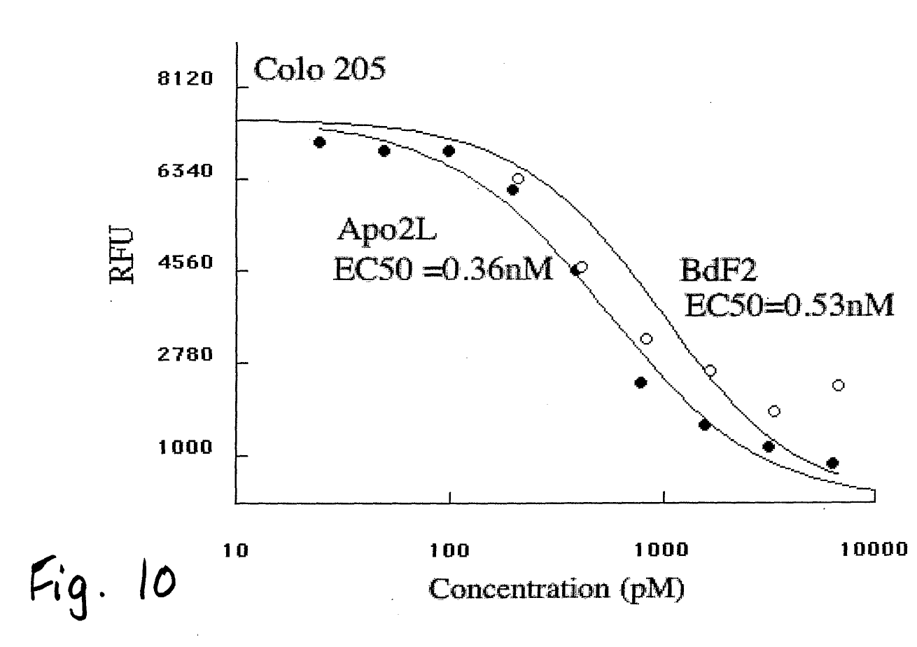

The results of the bioassay are shown in FIG. 10.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

TABLE 9

POLYPEPTIDE SEQUENCES OF REAGENTS USED IN
EXAMPLE ASSAYS

1. Human DR5-ECD polypeptide

(SEQ ID NO:154)

MSALLILALVGAAVADYKDDDDKLSALITQQDLAPQQRVAPQQKRSSPSE
GLCPPGHHISEDGRDCISCKYGQDYSTHWNDLLFCLRCTRCDSGEVELSP
CTTTRNTVCQCEEGTFREEDSPEMCRKCRTGCPRGMVKVGDCTPWSDIEC
VHKESGTKHSGEAPAVEETVTSSPGTPASPCSLS
2. Human DR4 IgG fusion polypeptide

(SEQ ID NO:155)

MAPPPARVHLGAFLAVTPNPGSAASGTEAAAATPSKVWGSSAGRIEPRGG
GRGALPTSMGQHGPSARARAGRAPGPRPAREASPRLRVHKTFKFVVVGVL
LQVVPSSAATIKLHDQSIGTQQWEHSPLGELCPPGSHRSERPGACNRCTE
GVGYTNASNNLFACLPCTACKSDEEERSPCTTTRNTACQCKPGTFRNDNS
AEMCRKCSTGCPRGMVKVKDCTPWSDIECVHKESGNGHNDKTHTCPPCPA
PELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEA
LHNHYTQKSLSLSPGK
3. Human DR5-IgG fusion polypeptide

(SEQ ID NO:156)

MEQRGQNAPAASGARKRHGPGPREARGARPGLRVPKTLVLVVAAVLLLVS
AESALITQQDLAPQQRAAPQQKRSSPSEGLCPPGHHISEDGRDCISCKYG
QDYSTHWNDLLFCLRCTRCDSGEVELSPCTTTRNTVCQCEEGTFREEDSP
EMCRKCRTGCPRGMVKVGDCTPWSDIECVHKESGLAFQDKTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEAL
HNHYTQKSLSLSPGK
4. Cynomolgous DR4-IgG fusion polypeptide

(SEQ ID NO:157)

MGQQGPSAQARAGRVVGPRSAQGASPGLRVHKTLKFVVVGVLLQVVPGSA
ATIKVHDQSVGTQQWEHSPLGELCPPGSHRSEHSGACNQCTEGVGYTSAS
NNLFSCLPCTACKSDEEERSACTRTRNTACQCKPGTFRNDDSAEMCRKCS
TGCPRGKVKVKDCTPWSDIECVHNESGNGHNVWAILIVTVVILVVLLLLV
AVLMFCRRIGSGCGGNPKCMHRVFLWCLGLLRGPGAEDNAHNMILNHGDS
LSTFISEQQMESQEPADLTGVTVQSPGEAQCLLGPAEPEGSQRRRLLVPA
NGADPTETMMLFFDNFADIVPFNSWDQLMRQLGLTNNEIHMVRADTAGPG
DALYAMLMKWVNKTGQDASIHTLLDALERIGERHAKERIQDLLVDSGKFI
YVEDGTGSAVSLE
5. Cynomolgous DR5-IgG fusion polypeptide

(SEQ ID NO:158)

MGQLRQSAPAASVARKGRGPGPREARGARPGLRVLKTLVLVVAAARVLLS
VSADCAPITRQSLDPQRRAAPQQKRSSPTEGLCPPGHHISEDSRECISCK
YGQDYSTHWNDFLFCLRCTKCDSGEVEVNSCTTTRNTVCQCEEGTFREED
SPEICRKCRTGCPRGMVKVKDCTPWSDIECVHKESGIIIGVIVLVVIVVV
TVIVWKTSLWKKVLPYLKGVCSGDGGDPEHVDSSSHSPQRPGAEDNALNE
IVSIVQPSQVPEQEMEVQEPAEQTDVNTLSPGESEHLLEPAKAEGPQRRG
QLVPVNENDPTETLRQCFDDFAAIVPFDAWEPLVRQLGLTNNEIKVAKAE
AASSRDTLYVMLIKWVNKTGRAASVNTLLDALETLEERLAKQKIQDRLLS
SGKFMYLEDNADSATS

What is claimed is:

1. An isolated anti-DR5 antibody comprising one or more amino acid sequences set forth in FIG. 6, 7 or 8.

2. An isolated anti-DR5 antibody, comprising a heavy chain and a light chain, wherein the heavy chain comprises a variable region comprising one or more amino acid sequences set forth in FIG. 6, 7 or 8.

3. The antibody of claim 2, wherein the heavy chain and the light chain are connected by a flexible linker to form a single-chain antibody.

4. The antibody of claim 3, which is a single-chain Fv antibody.

5. The antibody of claim 2, which is a Fab antibody.

6. The antibody of claim 2, which is fully human.

7. The antibody of claim 1 or claim 2 which specifically binds DR5 receptor and does not bind DR4 receptor, DcR1 receptor or DcR2 receptor.

8. The antibody of claim 1 or claim 2 which induces apoptosis in at least one type of mammalian cancer cells.

9. The antibody of claim 1 or claim 2 which blocks or inhibits binding of Apo-2 ligand to DR5 receptor.

10. A composition comprising an antibody of any of claims 1 to 9 and a carrier.

11. A method of treating a disorder in a mammal, comprising administering the composition of claim 10.

12. The method of claim 11, wherein the disorder is an immune-related disorder.

13. The method of claim 11, wherein the disorder is cancer.


SEQUENCE LISTING (484 sequences)

SEQ ID NO: 1 - protein, 281 aa, Homo sapiens [sequence omitted]

SEQ ID NO: 2 - DNA, 1042 bp, Homo sapiens
  CDS (91)..(933)
  modified_base (447)..(447): a, c, g, t, unknown or other
tttcctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtccncagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaa

SEQ ID NO: 3 - protein, 468 aa, Homo sapiens
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSERPGACNRCTEGVGYTNASNNLFACLPCTACKSDEEERSPCTTTRNTACQCKPGTFRNDNSAEMCRKCSTGCPRGMVKVKDCTPWSDIECVHKESGNGHNIWVILVVTLVVPLLLVAVLIVCCCIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAKEKIQDLLVDSGKFIYLEDGTGSAVSLE

SEQ ID NO: 4 - DNA, 1407 bp, Homo sapiens
  CDS (1)..(1404)
atggcgccaccaccagctagagtacatctaggtgcgttcctggcagtgactccgaatcccgggagcgcagcgagtgggacagaggcagccgcggccacacccagcaaagtgtggggctcttccgcggggaggattgaaccacgaggcgggggccgaggagcgctccctacctccatgggacagcacggacccagtgcccgggcccgggcagggcgcgccccaggacccaggccggcgcgggaagccagccctcggctccgggtccacaagaccttcaagtttgtcgtcgtcggggtcctgctgcaggtcgtacctagctcagctgcaaccatcaaacttcatgatcaatcaattggcacacagcaatgggaacatagccctttgggagagttgtgtccaccaggatctcatagatcagaacgtcctggagcctgtaaccggtgcacagagggtgtgggttacaccaatgcttccaacaatttgtttgcttgcctcccatgtacagcttgtaaatcagatgaagaagagagaagtccctgcaccacgaccaggaacacagcatgtcagtgcaaaccaggaactttccggaatgacaattctgctgagatgtgccggaagtgcagcacagggtgccccagagggatggtcaaggtcaaggattgtacgccctggagtgacatcgagtgtgtccacaaagaatcaggcaatggacataatatatgggtgattttggttgtgactttggttgttccgttgctgttggtggctgtgctgattgtctgttgttgcatcggctcaggttgtggaggggaccccaagtgcatggacagggtgtgtttctggcgcttgggtctcctacgagggcctggggctgaggacaatgctcacaacgagattctgagcaacgcagactcgctgtccactttcgtctctgagcagcaaatggaaagccaggagccggcagatttgacaggtgtcactgtacagtccccaggggaggcacagtgtctgctgggaccggcagaagctgaagggtctcagaggaggaggctgctggttccagcaaatggtgctgaccccactgagactctgatgctgttctttgacaagtttgcaaacatcgtgccctttgactcctgggaccagctcatgaggcagctggacctcacgaaaaatgagatcgatgtggtcagagctggtacagcaggcccaggggatgccttgtatgcaatgctgatgaaatgggtcaacaaaactggacggaacgcctcgatccacaccctgctggatgccttggagaggatggaagagagacatgcaaaagagaagattcaggacctcttggtggactctggaaagttcatctacttagaagatggcacaggctctgccgtgtccttggagtga

SEQ ID NO: 5 - protein, 411 aa, Homo sapiens [sequence omitted]

SEQ ID NO: 6 - protein, 440 aa, Homo sapiens [sequence omitted]

SEQ ID NO: 7 - DNA, 6412 bp, Artificial Sequence
  Description of Artificial Sequence Synthetic nucleotide sequence
gaattcaacttctccatactttggataaggaaatacagacatgaaaaatctcattgctgagttgttatttaagcttgcccaaaaagaagaagagtcgaatgaactgtgtgcgcaggtagaagctttggagattatcgtcactgcaatgcttcgcaatatggcgcaaaatgaccaacagcggttgattgatcaggtagagggggcgctgtacgaggtaaagcccgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtaactagtacgcaagttcacgtaaaaagggtatgtagaggttgaggtgattttatgaaaaagaatatcgcatttcttcttgcatctatgttcgttttttctattgctacaaatgcctatgcatccgatatccagatgacccagtccccgagctccctgtccgcctctgtgggcgatagggtcaccatcacctgccgtgccagtcaggatgtgaatactgctgtagcctggtatcaacagaaaccaggaaaagctccgaagcttctgatttactcggcatccttcctctactctggagtcccttctcgcttctctggtagccgttccgggacggatttcactctgaccatcagcagtctgcagccggaagacttcgcaacttattactgtcagcaacattatactactcctcccacgttcggacagggtaccaaggtggagatcaaatcggatatgccgatggctgatccgaaccgtttccgcggtaagaacctggtttttcattctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctacaaatgaacagcttaagagctgaggacactgccgtctattattgtagccgctggggaggggacggcttctatgctatggactactggggtcaaggaacactagtcaccgtctccagcacagctccgccggcaccagcaccagaactgctgggcggccgcatgaaacagctagaggacaaggtcgaagagctactctccaagaactaccacctagagaatgaagtggcaagactcaaaaaacttgtcggggagcgcggaaagcttagtggcggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttggctagcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcaggatccggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggctatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccggatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatctacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatc

SEQ ID NO: 8 - DNA, 7060 bp, Artificial Sequence
  Description of Artificial Sequence Synthetic nucleotide sequence
gaattcaacttctccatactttggataaggaaatacagacatgaaaaatctcattgctgagttgttatttaagcttgcccaaaaagaagaagagtcgaatgaactgtgtgcgcaggtagaagctttggagattatcgtcactgcaatgcttcgcaatatggcgcaaaatgaccaacagcggttgattgatcaggtagagggggcgctgtacgaggtaaagcccgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtaactagtacgcaagttcacgtaaaaagggtatgtagaggttgaggtgattttatgaaaaagaatatcgcatttcttcttgcatctatgttcgttttttctattgctacaaatgcctatgcagatatccagatgacccagtccccgagctccctgtccgcctctgtgggcgatagggtcaccatcacctgccgtgccagtcaggatgtgtccactgctgtagcctggtatcaacagaaaccaggaaaagctccgaagcttctgatttactcggcatccttcctctactctggagtcccttctcgcttctctggtagcggttccgggacggatttcactctgaccatcagcagtctgcagccggaagacttcgcaacttattactgtcagcaatcttatactactcctcccacgttcggacagggtaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggtgccagctccggtatggctgatccgaaccgtttccgcggtaaggacctggcataactcgaggctgatcctctacgccggacgcatcgtggccctagtacgcaagttcacgtaaaaagggtaactagaggttgaggtgattttatgaaaaagaatatcgcatttcttcttgcatctatgttcgttttttctattgctacaaacgcgtacgctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctacaaatgaacagcttaagagctgaggacactgccgtctattattgtagccgctggggaggggacggcttctatgctatggactactggggtcaaggaacactagtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacctcagtggcggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttggctagcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcaggatccggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggctatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccggatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatctacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatc

SEQ ID NO: 9 - protein, 80 aa, Artificial Sequence
  Description of Artificial Sequence Synthetic peptide
GFTIGGSTIHXXXXXXXXXXXXVATIYPTYGYTYYAXXXXXXXXXXXXXXXXXXXXXXXXXXXXXXXXXXXSREGKYAMD

SEQ ID NO: 10 - protein, 80 aa, Artificial Sequence
  Description of Artificial Sequence Synthetic peptide
GFSIAKYAIHXXXXXXXXXXXXVALIAPSAGATNYAXXXXXXXXXXXXXXXXXXXXXXXXXXXXXXXXXXXSRSAWYAMD

SEQ ID NO: 11 - protein, 80 aa, Artificial Sequence
  Description of Artificial Sequence Synthetic peptide
GFSIGGSIIHXXXXXXXXXXXXVATIFPTDGYTNYAXXXXXXXXXXXXXXXXXXXXXXXXXXXXXXXXXXXSRKNRYAMD

SEQ ID NO: 12 - protein, 81 aa, Artificial Sequence
  Description of Artificial Sequence Synthetic peptide
GFTIRRTVIHXXXXXXXXXXXXVASIAPYDGDTAYAXXXXXXXXXXXXXXXXXXXXXXXXXXXXXXXXXXXSRGGWFYAMD

SEQ ID NO: 13 - protein, 81 aa, Artificial Sequence
  Description of Artificial Sequence Synthetic peptide
GFSIEATSIHXXXXXXXXXXXXVAMISPSTGTTTADXXXXXXXXXXXXXXXXXXXXXXXXXXXXXXXXXXXRAATRSYAMD

SEQ ID NO: 14 - protein, 81 aa, Artificial Sequence
  Description of Artificial Sequence Synthetic peptide
GFSIKGSVIHXXXXXXXXXXXXVARIYPXXRPXTRYXXXXXXXXXXXXXXXXXXXXXXXXXXXXXXXXXXXCSRAGIYAMD

SEQ ID NO: 15 - protein, 82 aa, Artificial Sequence
  Description of Artificial Sequence Synthetic peptide
GFTISNSIIHXXXXXXXXXXXXVAGIAPYNGDTTYAXXXXXXXXXXXXXXXXXXXXXXXXXXXXXXXXXXXSRAYSRQYAMD

SEQ ID NO: 16 - protein, 82 aa, Artificial Sequence
  Description of Artificial Sequence Synthetic peptide
GFSISRTAIHXXXXXXXXXXXXVASIVPAYADTYYAXXXXXXXXXXXXXXXXXXXXXXXXXXXXXXXXXXXSRSSRSMYTMD

SEQ ID NO: 17 - protein, 82 aa, Artificial Sequence
  Description of Artificial Sequence Synthetic peptide
GFSITATVIHXXXXXXXXXXXXVARIAPHSGDTTYAXXXXXXXXXXXXXXXXXXXXXXXXXXXXXXXXXXXSRAYYREYAMD

SEQ ID NO: 18 - protein, 82 aa, Artificial Sequence
  Description of Artificial Sequence Synthetic peptide
GFSYXFCYNHXXXXXXXXXXXXVAGIVPATGNTYYAXXXXXXXXXXXXXXXXXXXXXXXXXXXXXXXXXXXSRGRYAMYAMD

SEQ ID NO: 19 - protein, 82 aa, Artificial Sequence
  Description of Artificial Sequence Synthetic peptide
GFSIRTTAIHXXXXXXXXXXXXVAGIIPYTGSTSYAXXXXXXXXXXXXXXXXXXXXXXXXXXXXXXXXXXXSRGSRSEYAMD

SEQ ID NO: 20 - protein, 82 aa, Artificial Sequence
  Description of Artificial Sequence Synthetic peptide
GFTITSSVIHXXXXXXXXXXXXVAGIAPYNGTTDYAXXXXXXXXXXXXXXXXXXXXXXXXXXXXXXXXXXXSRAWYAQYAMD

SEQ ID NO: 21 - protein, 82 aa, Artificial Sequence
  Description of Artificial Sequence Synthetic peptide
GFSIGSSGIHXXXXXXXXXXXXVARIFPHSGATTYAXXXXXXXXXXXXXXXXXXXXXXXXXXXXXXXXXXXSRSWYAEYAMD

SEQ ID NO: 22 - protein, 82 aa, Artificial Sequence
  Description of Artificial Sequence Synthetic peptide
GFSITSSGIHXXXXXXXXXXXXVALIYPHSGATSYAXXXXXXXXXXXXXXXXXXXXXXXXXXXXXXXXXXXSRSWKAEYAMD

SEQ ID NO: 23 - protein, 82 aa, Artificial Sequence
  Description of Artificial Sequence Synthetic peptide
GFTIRRSGIHXXXXXXXXXXXXVAGIVPAAGNTDYAXXXXXXXXXXXXXXXXXXXXXXXXXXXXXXXXXXXSRSWWEHYAMD

SEQ ID NO: 24 - protein, 82 aa, Artificial Sequence
  Description of Artificial Sequence Synthetic peptide
GFSIASTVIHXXXXXXXXXXXXVATIAPYNGNTTYAXXXXXXXXXXXXXXXXXXXXXXXXXXXXXXXXXXXSRARYSMYAMD

SEQ ID NO: 25 - protein, 82 aa, Artificial Sequence
  Description of Artificial Sequence Synthetic peptide
GFSIRTTAIHXXXXXXXXXXXXVAWIIPYTGSTSYAXXXXXXXXXXXXXXXXXXXXXXXXXXXXXXXXXXXSRGSRSEYAMD

SEQ ID NO: 26 - protein, 82 aa, Artificial Sequence
  Description of Artificial Sequence Synthetic peptide
GFTIGKSSIHXXXXXXXXXXXXFARIYPTYGATDYAXXXXXXXXXXXXXXXXXXXXXXXXXXXXXXXXXXXSRDWWTLYAMD

SEQ ID NO: 27 - protein, 83 aa, Artificial Sequence
  Description of Artificial Sequence Synthetic peptide
GFTIDSSGIHXXXXXXXXXXXXVARIFPSAGYTNYAXXXXXXXXXXXXXXXXXXXXXXXXXXXXXXXXXXXSRWSGSRRYAMD

SEQ ID NO: 28 - protein, 83 aa, Artificial Sequence
  Description of Artificial Sequence Synthetic peptide
GFSITRSAIHXXXXXXXXXXXXVASIIPYYGTTAYAXXXXXXXXXXXXXXXXXXXXXXXXXXXXXXXXXXXSRDGNSGHYAMD

SEQ ID NO: 29 - protein, 83 aa, Artificial Sequence
  Description of Artificial Sequence Synthetic peptide
GFTISSNGIHXXXXXXXXXXXXVATIIPYTGNTYYAXXXXXXXXXXXXXXXXXXXXXXXXXXXXXXXXXXXSRTYGWSGYAMD

SEQ ID NO: 30 - protein, 83 aa, Artificial Sequence
  Description of Artificial Sequence Synthetic peptide
GFSIGRSVIHXXXXXXXXXXXXVAGIVPSYGNTYYAXXXXXXXXXXXXXXXXXXXXXXXXXXXXXXXXXXXSRNYSGYFYAMD

SEQ ID NO: 31 - protein, 83 aa, Artificial Sequence
  Description of Artificial Sequence Synthetic peptide
GFSIRGNVIHXXXXXXXXXXXXVAGIVPHAGATYYAXXXXXXXXXXXXXXXXXXXXXXXXXXXXXXXXXXXSRGYSYTFYAMD

SEQ ID NO: 32 - protein, 83 aa, Artificial Sequence
  Description of Artificial Sequence Synthetic peptide
GFSIEEYAIHXXXXXXXXXXXXVAMIYPNYGATTYAXXXXXXXXXXXXXXXXXXXXXXXXXXXXXXXXXXXSRNYAGALYAMD

SEQ ID NO: 33 - protein, 83 aa, Artificial Sequence
  Description of Artificial Sequence Synthetic peptide
GFTIARSGIHXXXXXXXXXXXXVAWIVPAYGSTSYAXXXXXXXXXXXXXXXXXXXXXXXXXXXXXXXXXXXSRSATGEVYAMD

SEQ ID NO: 34 - protein, 84 aa, Artificial Sequence
  Description of Artificial Sequence Synthetic peptide
GFSITSTGIHXXXXXXXXXXXXVAGIYPHAGSTTYAXXXXXXXXXXXXXXXXXXXXXXXXXXXXXXXXXXXSRGSYKAWFYAMD

SEQ ID NO: 35 - protein, 84 aa, Artificial Sequence
  Description of Artificial Sequence Synthetic peptide
GFTITGSGIHXXXXXXXXXXXXVAWIDPAAGATAYAXXXXXXXXXXXXXXXXXXXXXXXXXXXXXXXXXXXSREGSGWATYAMD

SEQ ID NO: 36 - protein, 85 aa, Artificial Sequence
  Description of Artificial Sequence Synthetic peptide
GFSIGGYAIHXXXXXXXXXXXXVARINPNSGSTYYAXXXXXXXXXXXXXXXXXXXXXXXXXXXXXXXXXXXSRGAGYSKSAYAMD

SEQ ID NO: 37 - protein, 85 aa, Artificial Sequence
  Description of Artificial Sequence Synthetic peptide
GFSIEGSVIHXXXXXXXXXXXXVAMIIPYTGDTSYAXXXXXXXXXXXXXXXXXXXXXXXXXXXXXXXXXXXSRGEATWRRAYAMD

SEQ ID NO: 38 - protein, 85 aa, Artificial Sequence
  Description of Artificial Sequence Synthetic peptide
GFTIGGTVIHXXXXXXXXXXXXVAMIYPDNGYTNYAXXXXXXXXXXXXXXXXXXXXXXXXXXXXXXXXXXXSRNDYSGTALYAMD

SEQ ID NO: 39 - protein, 85 aa, Artificial Sequence
  Description of Artificial Sequence Synthetic peptide
GFSIGRYGIHXXXXXXXXXXXXVASIAPSDGATSYAXXXXXXXXXXXXXXXXXXXXXXXXXXXXXXXXXXXSRGAGYSYTLYAMD

SEQ ID NO: 40 - protein, 85 aa, Artificial Sequence
  Description of Artificial Sequence Synthetic peptide
GFSIDKYSIHXXXXXXXXXXXXVARIIPYTGATTYAXXXXXXXXXXXXXXXXXXXXXXXXXXXXXXXXXXXSRGGTSWSRLYAMD

SEQ ID NO: 41 - protein, 85 aa, Artificial Sequence
  Description of Artificial Sequence Synthetic peptide
GFSIKTSAIHXXXXXXXXXXXXVAGIVPTAGYTYYAXXXXXXXXXXXXXXXXXXXXXXXXXXXXXXXXXXXSRGDGTWGKLYAMD

SEQ ID NO: 42 - protein, 85 aa, Artificial Sequence
  Description of Artificial Sequence Synthetic peptide
GFSIKTSAIHXXXXXXXXXXXXFAGIVPTAGYTYYAXXXXXXXXXXXXXXXXXXXXXXXXXXXXXXXXXXXSRGDGTWGKLYAMD

SEQ ID NO: 43 - protein, 85 aa, Artificial Sequence
  Description of Artificial Sequence Synthetic peptide
GFSIAGSGIHXXXXXXXXXXXXVAGIAPASGSTNYAXXXXXXXXXXXXXXXXXXXXXXXXXXXXXXXXXXXSRRAGYSYTLYAMD

SEQ ID NO: 44 - protein, 85 aa, Artificial Sequence
  Description of Artificial Sequence Synthetic peptide
GFSIATSVIHXXXXXXXXXXXXVARIIPNNGSTAYAXXXXXXXXXXXXXXXXXXXXXXXXXXXXXXXXXXXSRAAARRSYMYAMD

SEQ ID NO: 45 - protein, 85 aa, Artificial Sequence
  Description of Artificial Sequence Synthetic peptide
GFSIGRSIIHXXXXXXXXXXXXFVVISPYSGYTNYAXXXXXXXXXXXXXXXXXXXXXXXXXXXXXXXXXXXSRGGTSYRSMYAMD

SEQ ID NO: 46 - protein, 85 aa, Artificial Sequence
  Description of Artificial Sequence Synthetic peptide
GFTIDSNDIHXXXXXXXXXXXXVARITPYTGATYYAXXXXXXXXXXXXXXXXXXXXXXXXXXXXXXXXXXXSRNRNSWAWRYAMD

SEQ ID NO: 47 - protein, 85 aa, Artificial Sequence
  Description of Artificial Sequence Synthetic peptide
GFSIAAYAIHXXXXXXXXXXXXVATIIPANGDTNYAXXXXXXXXXXXXXXXXXXXXXXXXXXXXXXXXXXXSRGAGRSYTRYAMD

SEQ ID NO: 48 - protein, 85 aa, Artificial Sequence
  Description of Artificial Sequence Synthetic peptide
GFTIDRNDIHXXXXXXXXXXXXVARITPYTGATDYAXXXXXXXXXXXXXXXXXXXXXXXXXXXXXXXXXXXSRNRNTWTRRYAMD

SEQ ID NO: 49 - protein, 85 aa, Artificial Sequence
  Description of Artificial Sequence Synthetic peptide
GFSIGEYVIHXXXXXXXXXXXXVARIIPYDGSTAYAXXXXXXXXXXXXXXXXXXXXXXXXXXXXXXXXXXXSRGWARWSRRYAMD

SEQ ID NO: 50 - protein, 85 aa, Artificial Sequence
  Description of Artificial Sequence Synthetic peptide
GFSIDKSVIPXXXXXXXXXXXXVARIIPAYGTTYYAXXXXXXXXXXXXXXXXXXXXXXXXXXXXXXXXXXXSRGGNSYTTRYAMD

SEQ ID NO: 51 - protein, 85 aa, Artificial Sequence
  Description of Artificial Sequence Synthetic peptide
GFSITDSVIHXXXXXXXXXXXXVARIDPPTGATYYAXXXXXXXXXXXXXXXXXXXXXXXXXXXXXXXXXXXSRSSNSWTRRYAMD

SEQ ID NO: 52 - protein, 85 aa, Artificial Sequence
  Description of Artificial Sequence Synthetic peptide
GFSISNYIIHXXXXXXXXXXXXVARIDPTNGNTYYAXXXXXXXXXXXXXXXXXXXXXXXXXXXXXXXXXXXSRAWATWGRRYAMD

SEQ ID NO: 53 - protein, 85 aa, Artificial Sequence
  Description of Artificial Sequence Synthetic peptide
GFSIEASVIHXXXXXXXXXXXXVARIIPYTGNTNYAXXXXXXXXXXXXXXXXXXXXXXXXXXXXXXXXXXXSRSSASWKSRYAMD

SEQ ID NO: 54 - protein, 85 aa, Artificial Sequence
  Description of Artificial Sequence Synthetic peptide
GFTIETSTIHXXXXXXXXXXXXVAGIIPYTGTTDYAXXXXXXXXXXXXXXXXXXXXXXXXXXXXXXXXXXXSRGGASWTRRYAMD

SEQ ID NO: 55 - protein, 85 aa, Artificial Sequence
  Description of Artificial Sequence Synthetic peptide
GFSIAGNTIHXXXXXXXXXXXXVAGITPATGYTNYAXXXXXXXXXXXXXXXXXXXXXXXXXXXXXXXXXXXSRGGGRYAWRYAMD

SEQ ID NO: 56 - protein, 85 aa, Artificial Sequence
  Description of Artificial Sequence Synthetic peptide
GFTIADSNIHXXXXXXXXXXXXVARIFPHTGDTYYAXXXXXXXXXXXXXXXXXXXXXXXXXXXXXXXXXXXSRSAGTWSRRYAMD

SEQ ID NO: 57 - protein, 85 aa, Artificial Sequence
  Description of Artificial Sequence Synthetic peptide
GFTIEEYNIHXXXXXXXXXXXXFARIVPYTGYTYYAXXXXXXXXXXXXXXXXXXXXXXXXXXXXXXXXXXXSRSTASRSSRYAMD

SEQ ID NO: 58 - protein, 85 aa, Artificial Sequence
  Description of Artificial Sequence Synthetic peptide
GFSIADTVIHXXXXXXXXXXXXVARIYPYAGSTNYAXXXXXXXXXXXXXXXXXXXXXXXXXXXXXXXXXXXSRSANSWSTRYAMD

SEQ ID NO: 59 - protein, 85 aa, Artificial Sequence
  Description of Artificial Sequence Synthetic peptide
GFTIGGTTIHXXXXXXXXXXXXVASINPHSGSTAYAXXXXXXXXXXXXXXXXXXXXXXXXXXXXXXXXXXXSRGAGYSYTRYAMD

SEQ ID NO: 60 - protein, 85 aa, Artificial Sequence
  Description of Artificial Sequence Synthetic peptide
GFTIGNSTIHXXXXXXXXXXXXVAGIIPASGSTAYAXXXXXXXXXXXXXXXXXXXXXXXXXXXXXXXXXXXSRGGGRWSRRYAMD

SEQ ID NO: 61 - protein, 85 aa, Artificial Sequence
  Description of Artificial Sequence Synthetic peptide
GFSIDKSVIHXXXXXXXXXXXXVARIIPAYGTTYYAXXXXXXXXXXXXXXXXXXXXXXXXXXXXXXXXXXXSRGGNSYTTRYAMD

SEQ ID NO: 62 - protein, 85 aa, Artificial Sequence
  Description of Artificial Sequence Synthetic peptide
GFSIDEYVIHXXXXXXXXXXXXVARIDPYSGATYYAXXXXXXXXXXXXXXXXXXXXXXXXXXXXXXXXXXXSRATGTWSSRYAMD

SEQ ID NO: 63 - protein, 85 aa, Artificial Sequence
  Description of Artificial Sequence Synthetic peptide
GFSIDGSSIHXXXXXXXXXXXXVARITPYSGNTTYAXXXXXXXXXXXXXXXXXXXXXXXXXXXXXXXXXXXSRGSAYYSSTYAMD

SEQ ID NO: 64 - protein, 85 aa, Artificial Sequence
  Description of Artificial Sequence Synthetic peptide
GFSIDRYVIHXXXXXXXXXXXXVARIAPYSGDTNYAXXXXXXXXXXXXXXXXXXXXXXXXXXXXXXXXXXXSRGANSRSRVYAMD

SEQ ID NO: 65 - protein, 85 aa, Artificial Sequence
  Description of Artificial Sequence Synthetic peptide
GFTIRTNAIHXXXXXXXXXXXXVARINPYSGYTTYAXXXXXXXXXXXXXXXXXXXXXXXXXXXXXXXXXXXSRWGNTETAVYAMD

SEQ ID NO: 66 - protein, 85 aa, Artificial Sequence
  Description of Artificial Sequence Synthetic peptide
GFTIRTNAIHXXXXXXXXXXXXFARINPYSGYTTYAXXXXXXXXXXXXXXXXXXXXXXXXXXXXXXXXXXXSRWGNTETAVYAMD

SEQ ID NO: 67 - protein, 85 aa, Artificial Sequence
  Description of Artificial Sequence Synthetic peptide
GFSIGNSAIHXXXXXXXXXXXXVARINPHTGATTYAXXXXXXXXXXXXXXXXXXXXXXXXXXXXXXXXXXXSRWGADSWAVYAMD

SEQ ID NO: 68 - protein, 86 aa, Artificial Sequence
  Description of Artificial Sequence Synthetic peptide
GFSITNYVIHXXXXXXXXXXXXVAGITPHSGYTAYAXXXXXXXXXXXXXXXXXXXXXXXXXXXXXXXXXXXSRGRKASYRARYAMD

SEQ ID NO: 69 - protein, 86 aa, Artificial Sequence
  Description of Artificial Sequence Synthetic peptide
GFTIDETTIHXXXXXXXXXXXXVAMIAPAYGATTYAXXXXXXXXXXXXXXXXXXXXXXXXXXXXXXXXXXXSRGKSWRACEYYAMD

SEQ ID NO: 70 - protein, 86 aa, Artificial Sequence
  Description of Artificial Sequence Synthetic peptide
GFTIDETTIHXXXXXXXXXXXXVAMIAPAYGATTYAXXXXXXXXXXXXXXXXXXXXXXXXXXXXXXXXXXXSRGKSWRAWEYYAMD

SEQ ID NO: 71 - protein, 86 aa, Artificial Sequence
  Description of Artificial Sequence Synthetic peptide
GFSINNYVIHXXXXXXXXXXXXVAGIIPYTGNTYYAXXXXXXXXXXXXXXXXXXXXXXXXXXXXXXXXXXXSRGRAATYTGQYAMD

SEQ ID NO: 72 - protein, 86 aa, Artificial Sequence
  Description of Artificial Sequence Synthetic peptide
GFSIDGYAIHXXXXXXXXXXXXVARIYPASGATNYAXXXXXXXXXXXXXXXXXXXXXXXXXXXXXXXXXXXSRSGATYRGSRYAMD

SEQ ID NO: 73 - protein, 87 aa, Artificial Sequence
  Description of Artificial Sequence Synthetic peptide
GFSIGRYSIHXXXXXXXXXXXXVARIDPDAGATDYAXXXXXXXXXXXXXXXXXXXXXXXXXXXXXXXXXXXSRGGTKARYSELYAMD

SEQ ID NO: 74 - protein, 87 aa, Artificial Sequence
  Description of Artificial Sequence Synthetic peptide
GFSIDKTVIHXXXXXXXXXXXXVALISPYTGTTTYAXXXXXXXXXXXXXXXXXXXXXXXXXXXXXXXXXXXSRGGWSARGYSSYAMD

SEQ ID NO: 75 - protein, 87 aa, Artificial Sequence
  Description of Artificial Sequence Synthetic peptide
GFSIDKTVIHXXXXXXXXXXXXFALISPYTGTTTYAXXXXXXXXXXXXXXXXXXXXXXXXXXXXXXXXXXXSRGGWSAMGYSSYAMD

SEQ ID NO: 76 - protein, 89 aa, Artificial Sequence
  Description of Artificial Sequence Synthetic peptide
GFTIANTTIHXXXXXXXXXXXXFAGINPASGDTTYAXXXXXXXXXXXXXXXXXXXXXXXXXXXXXXXXXXXSRERYWTSGTTYGSYAMD

SEQ ID NO: 77 - protein, 90 aa, Artificial Sequence
  Description of Artificial Sequence Synthetic peptide
GFSIAGSIIHXXXXXXXXXXXXVAMIAPTSGNTAYAXXXXXXXXXXXXXXXXXXXXXXXXXXXXXXXXXXXSRSWSSWGWGSSTGRYAMD

SEQ ID NO: 78 - protein, 82 aa, Artificial Sequence
  Description of Artificial Sequence Synthetic peptide
GFTISATAIHXXXXXXXXXXXXVARITPSDGTTDYAXXXXXXXXXXXXXXXXXXXXXXXXXXXXXXXXXXXARQLTLSGGMD

SEQ ID NO: 79 - protein, 82 aa, Artificial Sequence
  Description of Artificial Sequence Synthetic peptide
GFTISDTAIHXXXXXXXXXXXXVASISPSSGATYYAXXXXXXXXXXXXXXXXXXXXXXXXXXXXXXXXXXXARLLSRSGAMD

SEQ ID NO: 80 - protein, 82 aa, Artificial Sequence
  Description of Artificial Sequence Synthetic peptide
GFTISATAIHXXXXXXXXXXXXVARITPSDGTTDYAXXXXXXXXXXXXXXXXXXXXXXXXXXXXXXXXXXXARQLTLSGVMD

SEQ ID NO: 81 - protein, 82 aa, Artificial Sequence; identical to SEQ ID NO: 79
  Description of Artificial Sequence Synthetic peptide

SEQ ID NO: 82 - protein, 82 aa, Artificial Sequence
  Description of Artificial Sequence Synthetic peptide
GFTINATAIHXXXXXXXXXXXXVARINPAGGNTYYAXXXXXXXXXXXXXXXXXXXXXXXXXXXXXXXXXXXARSLSLSGAMD

SEQ ID NO: 83 - protein, 82 aa, Artificial Sequence
  Description of Artificial Sequence Synthetic peptide
GFTINSTAIHXXXXXXXXXXXXVAFIYPSDGATDYAXXXXXXXXXXXXXXXXXXXXXXXXXXXXXXXXXXXARLLTRSGAMD

SEQ ID NO: 84 - protein, 82 aa, Artificial Sequence; identical to SEQ ID NO: 80
  Description of Artificial Sequence Synthetic peptide

SEQ ID NO: 85 - protein, 82 aa, Artificial Sequence
  Description of Artificial Sequence Synthetic peptide
GFTITSTAIHXXXXXXXXXXXXVAYITPYSGYTYYAXXXXXXXXXXXXXXXXXXXXXXXXXXXXXXXXXXXARSFSWRGVMD

SEQ ID NO: 86 - protein, 82 aa, Artificial Sequence
  Description of Artificial Sequence Synthetic peptide
GFTINATAIHXXXXXXXXXXXXVAFISPTGGATNYAXXXXXXXXXXXXXXXXXXXXXXXXXXXXXXXXXXXARALTISGVMD

SEQ ID NO: 87 - protein, 82 aa, Artificial Sequence; identical to SEQ ID NO: 79
  Description of Artificial Sequence Synthetic peptide

SEQ ID NO: 88 - protein, 82 aa, Artificial Sequence
  Description of Artificial Sequence Synthetic peptide
GFTINSTAIHXXXXXXXXXXXXVARISPASGATYYAXXXXXXXXXXXXXXXXXXXXXXXXXXXXXXXXXXXARAATLRGVMD

SEQ ID NO: 89 - protein, 82 aa, Artificial Sequence
  Description of Artificial Sequence Synthetic peptide
GFTITSTAIHXXXXXXXXXXXXVAYISPYSGYTYYAXXXXXXXXXXXXXXXXXXXXXXXXXXXXXXXXXXXARSFSRGGVMD

SEQ ID NO: 90 - protein, 82 aa, Artificial Sequence
  Description of Artificial Sequence Synthetic peptide
GFTITRTSIHXXXXXXXXXXXXVAVINPTSGSTDYAXXXXXXXXXXXXXXXXXXXXXXXXXXXXXXXXXXXARLLGRWSGMD

SEQ ID NO: 91 - protein, 82 aa, Artificial Sequence
  Description of Artificial Sequence Synthetic peptide
GFTINNTWIHXXXXXXXXXXXXVAFISPASGATNYAXXXXXXXXXXXXXXXXXXXXXXXXXXXXXXXXXXXARLFSLSGAMD

SEQ ID NO: 92 - protein, 82 aa, Artificial Sequence
  Description of Artificial Sequence Synthetic peptide
GFTISDTAIHXXXXXXXXXXXXVARINPSSGSTYYAXXXXXXXXXXXXXXXXXXXXXXXXXXXXXXXXXXXARSLSRWYVMD

SEQ ID NO: 93 - protein, 82 aa, Artificial Sequence
  Description of Artificial Sequence Synthetic peptide
GFTITNSGIHXXXXXXXXXXXXVADIYPHSGSTDYAXXXXXXXXXXXXXXXXXXXXXXXXXXXXXXXXXXXARGRVAEYVMD

SEQ ID NO: 94 - protein, 82 aa, Artificial Sequence; identical to SEQ ID NO: 86
  Description of Artificial Sequence Synthetic peptide

SEQ ID NO: 95 - protein, 82 aa, Artificial Sequence
  Description of Artificial Sequence Synthetic peptide
GFTISGSYIHXXXXXXXXXXXXVARISPSGGYTYYAXXXXXXXXXXXXXXXXXXXXXXXXXXXXXXXXXXXARGFTYHGVMD

SEQ ID NO: 96 - protein, 82 aa, Artificial Sequence
  Description of Artificial Sequence Synthetic peptide
GFTITDSWIHXXXXXXXXXXXXVAWISPSSGSTYYAXXXXXXXXXXXXXXXXXXXXXXXXXXXXXXXXXXXARLLALSGAMD

SEQ ID NO: 97 - protein, 82 aa, Artificial Sequence; identical to SEQ ID NO: 86
  Description of Artificial Sequence Synthetic peptide

SEQ ID NO: 98 - protein, 82 aa, Artificial Sequence; identical to SEQ ID NO: 93
  Description of Artificial Sequence Synthetic peptide

SEQ ID NO: 99 - protein, 82 aa, Artificial Sequence
  Description of Artificial Sequence Synthetic peptide
GFTINNSDIHXXXXXXXXXXXXVAWISPHGGYTDYAXXXXXXXXXXXXXXXXXXXXXXXXXXXXXXXXXXXARRVSRSGAMD

SEQ ID NO: 100 - protein, 82 aa, Artificial Sequence
  Description of Artificial Sequence Synthetic peptide
GFTITGTGIHXXXXXXXXXXXXVAAINPSDGSTDYAXXXXXXXXXXXXXXXXXXXXXXXXXXXXXXXXXXXARLLSLSGAMD

SEQ ID NO: 101 - protein, 82 aa, Artificial Sequence
  Description of Artificial Sequence Synthetic peptide
GFTITSTSIHXXXXXXXXXXXXVAFISPTSGYTYYAXXXXXXXXXXXXXXXXXXXXXXXXXXXXXXXXXXXARAATRSYAMD

SEQ ID NO: 102 - protein, 82 aa, Artificial Sequence
  Description of Artificial Sequence Synthetic peptide
GFTITNSGIHXXXXXXXXXXXXVADIYPHSGSTDYAXXXXXXXXXXXXXXXXXXXXXXXXXXXXXXXXXXXARGRVVEYVMD

SEQ ID NO: 103 - protein, 82 aa, Artificial Sequence
  Description of Artificial Sequence Synthetic peptide
GFTITETSIHXXXXXXXXXXXXVADISPNDGNTDYAXXXXXXXXXXXXXXXXXXXXXXXXXXXXXXXXXXXARKLSVSGAMD

SEQ ID NO: 104 - protein, 82 aa, Artificial Sequence
  Description of Artificial Sequence Synthetic peptide
GFTISSTSIHXXXXXXXXXXXXVARIYPSDGDTNYAXXXXXXXXXXXXXXXXXXXXXXXXXXXXXXXXXXXARALTVRGAMD

SEQ ID NO: 105 - protein, 82 aa, Artificial Sequence
  Description of Artificial Sequence Synthetic peptide
GFTITDTSIHXXXXXXXXXXXXVAFINPNGGNTYYAXXXXXXXXXXXXXXXXXXXXXXXXXXXXXXXXXXXARLLTRAGAMD

SEQ ID NO: 106 - protein, 82 aa, Artificial Sequence
  Description of Artificial Sequence Synthetic peptide
GFTINATYIHXXXXXXXXXXXXVARISPSNGNTNYAXXXXXXXXXXXXXXXXXXXXXXXXXXXXXXXXXXXARALSRSSGMD

SEQ ID NO: 107 - protein, 82 aa, Artificial Sequence
  Description of Artificial Sequence Synthetic peptide
GFTITNSGIHXXXXXXXXXXXXVAGIYPYNGDTYYAXXXXXXXXXXXXXXXXXXXXXXXXXXXXXXXXXXXARTRFVYYVMD

SEQ ID NO: 108 - protein, 82 aa, Artificial Sequence
  Description of Artificial Sequence Synthetic peptide
GFTITGTAIHXXXXXXXXXXXXVARISPNGGSTNYAXXXXXXXXXXXXXXXXXXXXXXXXXXXXXXXXXXXARSLARTSGMD

SEQ ID NO: 109 - protein, 82 aa, Artificial Sequence
  Description of Artificial Sequence Synthetic peptide
GFTINSTAIHXXXXXXXXXXXXVAFISPSNGSTYYAXXXXXXXXXXXXXXXXXXXXXXXXXXXXXXXXXXXARQITLRGAMD

SEQ ID NO: 110 - protein, 81 aa, Artificial Sequence
  Description of Artificial Sequence Synthetic peptide
GFTINTSWIHXXXXXXXXXXXXVAWISPNGGYTNYAXXXXXXXXXXXXXXXXXXXXXXXXXXXXXXXXXXXARRRALGAMD

SEQ ID NO: 111 - protein, 83 aa, Artificial Sequence
  Description of Artificial Sequence Synthetic peptide
GFSIGKSGIHXXXXXXXXXXXXVAVIYPHDGNTAYAXXXXXXXXXXXXXXXXXXXXXXXXXXXXXXXXXXXARRLALVRMWMD

SEQ ID NO: 112 - protein, 83 aa, Artificial Sequence
  Description of Artificial Sequence Synthetic peptide
GFSIRRTDIHXXXXXXXXXXXXVARIYPNSGYTSYAXXXXXXXXXXXXXXXXXXXXXXXXXXXXXXXXXXXARNVRRRKPTFD

SEQ ID NO: 113 - protein, 83 aa, Artificial Sequence
  Description of Artificial Sequence Synthetic peptide
GFSIRKTDIHXXXXXXXXXXXXVARIYPNSGYTSYAXXXXXXXXXXXXXXXXXXXXXXXXXXXXXXXXXXXARNVRMRKPTLD

SEQ ID NO: 114 - protein, 83 aa, Artificial Sequence
  Description of Artificial Sequence Synthetic peptide
GFSIGKSGIHXXXXXXXXXXXXVAVIYPHDGNTAYAXXXXXXXXXXXXXXXXXXXXXXXXXXXXXXXXXXXARRLTLVRMWMD

SEQ ID NO: 115 - protein, 83 aa, Artificial Sequence
  Description of Artificial Sequence Synthetic peptide
GFSIGKSGIHXXXXXXXXXXXXVAVIYPHDGNTAYAXXXXXXXXXXXXXXXXXXXXXXXXXXXXXXXXXXXARRLSLVRMWMD

SEQ ID NO: 116 - DNA, 54 bp, Artificial Sequence
  Description of Artificial Sequence Synthetic oligonucleotide
tgtgcagcttctggcttcwccattrvnrvnwmyrntatacactgggtgcgtcag

SEQ ID NO: 117 - DNA, 66 bp, Artificial Sequence
  Description of Artificial Sequence Synthetic oligonucleotide
ggcctggaatgggttgcadbgattdhtccanmydmtggtdmtactdmttatgccgatagcgtcaag

SEQ ID NO: 118 - DNA, 50 bp, Artificial Sequence
  Description of Artificial Sequence Synthetic oligonucleotide
gccgtctattattgtagccgcdvkdvknnktacgctatggactactgggg

SEQ ID NO: 119 - DNA, 53 bp, Artificial Sequence
  Description of Artificial Sequence Synthetic oligonucleotide
gccgtctattattgtagccgcdvkdvkdvknnktacgctatggactactgggg

SEQ ID NO: 120 - DNA, 56 bp, Artificial Sequence
  Description of Artificial Sequence Synthetic oligonucleotide
gccgtctattattgtagccgcdvkdvkdvkdvknnktacgctatggactactgggg

SEQ ID NO: 121 - DNA, 59 bp, Artificial Sequence
  Description of Artificial Sequence Synthetic oligonucleotide
gccgtctattattgtagccgcdvkdvkdvkdvkdvknnktacgctatggactactgggg

SEQ ID NO: 122 - DNA, 62 bp, Artificial Sequence
  Description of Artificial Sequence Synthetic oligonucleotide
gccgtctattattgtagccgcdvkdvkdvkdvkdvkdvknnktacgctatggactactgggg

SEQ ID NO: 123 - DNA, 65 bp, Artificial Sequence
  Description of Artificial Sequence Synthetic oligonucleotide
gccgtctattattgtagccgcdvkdvkdvkdvkdvkdvkdvknnktacgctatggactactgggg

SEQ ID NO: 124 - DNA, 68 bp, Artificial Sequence
  Description of Artificial Sequence Synthetic oligonucleotide
gccgtctattattgtagccgcdvkdvkdvkdvkdvkdvkdvkdvknnktacgctatggactactgggg

SEQ ID NO: 125 - DNA, 71 bp, Artificial Sequence
  Description of Artificial Sequence Synthetic oligonucleotide
gccgtctattattgtagccgcdvkdvkdvkdvkdvkdvkdvkdvkdvknnktacgctatggactactgggg

SEQ ID NO: 126 - DNA, 74 bp, Artificial Sequence
  Description of Artificial Sequence Synthetic oligonucleotide
gccgtctattattgtagccgcdvkdvkdvkdvkdvkdvkdvkdvkdvkdvknnktacgctatggactactgggg

SEQ ID NO: 127 - DNA, 77 bp, Artificial Sequence
  Description of Artificial Sequence Synthetic oligonucleotide
gccgtctattattgtagccgcdvkdvkdvkdvkdvkdvkdvkdvkdvkdvkdvknnktacgctatggactactgggg

SEQ ID NO: 128 - DNA, 80 bp, Artificial Sequence
  Description of Artificial Sequence Synthetic oligonucleotide
gccgtctattattgtagccgcdvkdvkdvkdvkdvkdvkdvkdvkdvkdvkdvkdvknnktacgctatggactactgggg

SEQ ID NO: 129 - DNA, 83 bp, Artificial Sequence
  Description of Artificial Sequence Synthetic oligonucleotide
gccgtctattattgtagccgcdvkdvkdvkdvkdvkdvkdvkdvkdvkdvkdvkdvkdvknnktacgctatggactactgggg

SEQ ID NO: 130 - DNA, 51 bp, Artificial Sequence
  Description of Artificial Sequence Synthetic oligonucleotide
gcagcttctggcttcaccattavtrrtwmykmtatacactgggtgcgtcag

SEQ ID NO: 131 - DNA, 51 bp, Artificial Sequence
  Description of Artificial Sequence Synthetic oligonucleotide
gcagcttctggcttcaccattavtrrtwmykggatacactgggtgcgtcag

SEQ ID NO: 132 - DNA, 51 bp, Artificial Sequence
  Description of Artificial Sequence Synthetic oligonucleotide
gcagcttctggcttcaccattavtrvmwmykmtatacactgggtgcgtcag

SEQ ID NO: 133 - DNA, 51 bp, Artificial Sequence
  Description of Artificial Sequence Synthetic oligonucleotide
gcagcttctggcttcaccattavtrvmwmykggatacactgggtgcgtcag

SEQ ID NO: 134 - DNA, 71 bp, Artificial Sequence
  Description of Artificial Sequence Synthetic oligonucleotide
aagggcctggaatgggttgstdhtattwmtcctdmtrrcggtdmtactdactatgccgatagcgtcaaggg

SEQ ID NO: 135 - DNA, 72 bp, Artificial Sequence
  Description of Artificial Sequence Synthetic oligonucleotide
aagggcctggaatgggttgstdggattwmtcctdmtrrcggtdmtactdactatgccgatagcgtcaagggc

SEQ ID NO: 136 - DNA, 72 bp, Artificial Sequence
  Description of Artificial Sequence Synthetic oligonucleotide
aagggcctggaatgggttgstdhtattdmtcctnmtrrcggcdmtactdactatgccgatagcgtcaagggc

SEQ ID NO: 137 - DNA, 72 bp, Artificial Sequence
  Description of Artificial Sequence Synthetic oligonucleotide
aagggcctggaatgggttgstdggattdmtcctnmtrrcggcdmtactdactatgccgatagcgtcaagggc

SEQ ID NO: 138 - DNA, 60 bp, Artificial Sequence
  Description of Artificial Sequence Synthetic oligonucleotide
actgccgtctattattgtgctcgtnnsnnsnnsnnstacgbtatggactactggggtcaa

SEQ ID NO: 139 - DNA, 60 bp, Artificial Sequence
  Description of Artificial Sequence Synthetic oligonucleotide
actgccgtctattattgtgctcgtnnsnnsnnsnnsksggbtatggactactggggtcaa

SEQ ID NO: 140 - DNA, 63 bp, Artificial Sequence
  Description of Artificial Sequence Synthetic oligonucleotide
actgccgtctattattgtgctcgtnnsnnsnnsnnsnnstacgbtatggactactggggtcaa

SEQ ID NO: 141 - DNA, 63 bp, Artificial Sequence
  Description of Artificial Sequence Synthetic oligonucleotide
actgccgtctattattgtgctcgtnnsnnsnnsnnsnnsksggbtatggactactggggtcaa

SEQ ID NO: 142 - DNA, 66 bp, Artificial Sequence
  Description of Artificial Sequence Synthetic oligonucleotide
actgccgtctattattgtgcaaradvkdvkdvkdvkdvknnktacgctatggactactggggtcaa

SEQ ID NO: 143 - DNA, 66 bp, Artificial Sequence
  Description of Artificial Sequence Synthetic oligonucleotide
actgccgtctattattgtgcaaratggnvtdvkdvkdvkdvkdsggctatggactactggggtcaa

SEQ ID NO: 144 - DNA, 66 bp, Artificial Sequence
  Description of Artificial Sequence Synthetic oligonucleotide
actgccgtctattattgtgcaaradvkdvkdvkdvkdvkdvkksggctatggactactggggtcaa

SEQ ID NO: 145 - DNA, 69 bp, Artificial Sequence
  Description of Artificial Sequence Synthetic oligonucleotide
actgccgtctattattgtgcacgtdvkdvkdvkdvkdvkdvkdvktacgctatggactactggggtcaa

SEQ ID NO: 146 - DNA, 69 bp, Artificial Sequence
  Description of Artificial Sequence Synthetic oligonucleotide
actgccgtctattattgtgcacgtdvkdvkdvkdvkdvkdvkdvkdsggctatggactactggggtcaa

SEQ ID NO: 147 - DNA, 72 bp, Artificial Sequence
  Description of Artificial Sequence Synthetic oligonucleotide
actgccgtctattattgtgcacgtdvkdvkdvkdvkdvkdvkdvkdvktacgctatggactactggggtcaa

SEQ ID NO: 148 - DNA, 72 bp, Artificial Sequence
  Description of Artificial Sequence Synthetic oligonucleotide
actgccgtctattattgtgcacgtdvkdvkdvkdvkdvkdvkdvkdvkdsggctatggactactggggtcaa

SEQ ID NO: 149 - DNA, 54 bp, Artificial Sequence
  Description of Artificial Sequence Synthetic oligonucleotide
gccgtctattattgtgctcgcnnknnknnknnknnkwtkgactactggggtcaa

SEQ ID NO: 150 - DNA, 57 bp, Artificial Sequence
  Description of Artificial Sequence Synthetic oligonucleotide
gccgtctattattgtgctcgcnnknnknnknnknnknnkwtkgactactggggtcaa

SEQ ID NO: 151 - DNA, 60 bp, Artificial Sequence
  Description of Artificial Sequence Synthetic oligonucleotide
gccgtctattattgtgctcgcnnknnknnknnknnknnknnkwtkgactactggggtcaa

SEQ ID NO: 152 - DNA, 63 bp, Artificial Sequence
  Description of Artificial Sequence Synthetic oligonucleotide
gccgtctattattgtgctcgcnnknnknnknnknnknnknnknnkwtkgactactggggtcaa

SEQ ID NO: 153 - DNA, 66 bp, Artificial Sequence
  Description of Artificial Sequence Synthetic oligonucleotide
gccgtctattattgtgctcgcnnknnknnknnknnknnknnknnknnkwtkgactactggggtcaa

SEQ ID NO: 154 - protein, 184 aa, Homo sapiens [sequence omitted]

SEQ ID NO: 155 - protein, 466 aa, Artificial Sequence
  Description of Artificial Sequence Synthetic protein sequence
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSERPGACNRCTEGVGYTNASNNLFACLPCTACKSDEEERSPCTTTRNTACQCKPGTFRNDNSAEMCRKCSTGCPRGMVKVKDCTPWSDIECVHKESGNGHNDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 415 aa, Artificial Sequence
  Description of Artificial Sequence Synthetic protein sequence
MEQRGQNAPAASGARKRHGPGPREARGARPGLRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGLAFQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 413 aa, Artificial Sequence
  Description of Artificial Sequence Synthetic protein sequence
MGQQGPSAQARAGRVVGPRSAQGASPGLRVHKTLKFVVVGVLLQVVPGSAATIKVHDQSVGTQQWEHSPLGELCPPGSHRSEHSGACNQCTEGVGYTSASNNLFSCLPCTACKSDEEERSACTRTRNTACQCKPGTFRNDDSAEMCRKCSTGCPRGKVKVKDCTPWSDIECVHNESGNGHNVWAILIVTVVILVVLLLLVAVLMFCRRIGSGCGGNPKCMHRVFLWCLGLLRGPGAEDNAHNMILNHGDSLSTFISEQQMESQEPADLTGVTVQSPGEAQCLLGPAEPEGSQRRRLLVPANGADPTETMMLFFDNFADIVPFNSWDQLMRQLGLTNNEIHMVRADTAGPGDALYAMLMKWVNKTGQDASIHTLLDALERIGERHAKERIQDLLVDSGKFIYVEDGTGSAVSLE

SEQ ID NO: 158 - protein, 416 aa, Artificial Sequence
  Description of Artificial Sequence Synthetic protein sequence
MGQLRQSAPAASVARKGRGPGPREARGARPGLRVLKTLVLVVAAARVLLSVSADCAPITRQSLDPQRRAAPQQKRSSPTEGLCPPGHHISEDSRECISCKYGQDYSTHWNDFLFCLRCTKCDSGEVEVNSCTTTRNTVCQCEEGTFREEDSPEICRKCRTGCPRGMVKVKDCTPWSDIECVHKESGIIIGVIVLVVIVVVTVIVWKTSLWKKVLPYLKGVCSGDGGDPEHVDSSSHSPQRPGAEDNALNEIVSIVQPSQVPEQEMEVQEPAEQTDVNTLSPGESEHLLEPAKAEGPQRRGQLVPVNENDPTETLRQCFDDFAAIVPFDAWEPLVRQLGLTNNEIKVAKAEAASSRDTLYVMLIKWVNKTGRAASVNTLLDALETLEERLAKQKIQDRLLSSGKFMYLEDNADSATS

SEQ ID NO: 159 - protein, 255 aa, Artificial Sequence
  Description of Artificial Sequence Synthetic protein sequence
MKKNIAFLLASMFVFSIATNAYADIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGASSGMADPNRFRGKDLA

SEQ ID NO: 160 - protein, 409 aa, Artificial Sequence
  Description of Artificial Sequence Synthetic protein sequence
MKKNIAFLLASMFVFSIATNAYAEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 161 - protein, 10 aa, Artificial Sequence
  Description of Artificial Sequence Synthetic peptide
GFTIGGSTIH

SEQ ID NO: 162 - protein, 10 aa, Artificial Sequence
  Description of Artificial Sequence Synthetic peptide
GFSIAKYAIH

SEQ ID NO: 163 - protein, 10 aa, Artificial Sequence
  Description of Artificial Sequence Synthetic peptide
GFSIGGSIIH

SEQ ID NO: 164 - protein, 10 aa, Artificial Sequence
  Description of Artificial Sequence Synthetic peptide
GFTIRRTVIH

SEQ ID NO: 165 - protein, 10 aa, Artificial Sequence
  Description of Artificial Sequence Synthetic peptide
GFSIEATSIH

SEQ ID NO: 166 - protein, 10 aa, Artificial Sequence
  Description of Artificial Sequence Synthetic peptide
GFSIKGSVIH

SEQ ID NO: 167 - protein, 10 aa, Artificial Sequence
  Description of Artificial Sequence Synthetic peptide
GFTISNSIIH

SEQ ID NO: 168 - protein, 10 aa, Artificial Sequence
  Description of Artificial Sequence Synthetic peptide
GFSISRTAIH

SEQ ID NO: 169 - protein, 10 aa, Artificial Sequence
  Description of Artificial Sequence Synthetic peptide
GFSITATVIH

SEQ ID NO: 170 - protein, 10 aa, Artificial Sequence
  Description of Artificial Sequence Synthetic peptide
GFSYXFCYNH

SEQ ID NO: 171 - protein, 10 aa, Artificial Sequence
  Description of Artificial Sequence Synthetic peptide
GFSIRTTAIH

SEQ ID NO: 172 - protein, 10 aa, Artificial Sequence
  Description of Artificial Sequence Synthetic peptide
GFTITSSVIH

SEQ ID NO: 173 - protein, 10 aa, Artificial Sequence
  Description of Artificial Sequence Synthetic peptide
GFSIGSSGIH

SEQ ID NO: 174 - protein, 10 aa, Artificial Sequence
  Description of Artificial Sequence Synthetic peptide
GFSITSSGIH

SEQ ID NO: 175 - protein, 10 aa, Artificial Sequence
  Description of Artificial Sequence Synthetic peptide
GFTIRRSGIH

SEQ ID NO: 176 - protein, 10 aa, Artificial Sequence
  Description of Artificial Sequence Synthetic peptide
GFSIASTVIH

SEQ ID NO: 177 - protein, 10 aa, Artificial Sequence
  Description of Artificial Sequence Synthetic peptide
GFSIRTTAIH

SEQ ID NO: 178 - protein, 10 aa, Artificial Sequence
  Description of Artificial Sequence Synthetic peptide
GFTIGKSSIH

SEQ ID NO: 179 - protein, 10 aa, Artificial Sequence
  Description of Artificial Sequence Synthetic peptide
GFTIDSSGIH

SEQ ID NO: 180 - protein, 10 aa, Artificial Sequence
  Description of Artificial Sequence Synthetic peptide
GFSITRSAIH

SEQ ID NO: 181 - protein, 10 aa, Artificial Sequence
  Description of Artificial Sequence Synthetic peptide
GFTISSNGIH

SEQ ID NO: 182 - protein, 10 aa, Artificial Sequence
  Description of Artificial Sequence Synthetic peptide
GFSIGRSVIH

SEQ ID NO: 183 - protein, 10 aa, Artificial Sequence
  Description of Artificial Sequence Synthetic peptide
GFSIRGNVIH

SEQ ID NO: 184 - protein, 10 aa, Artificial Sequence
  Description of Artificial Sequence Synthetic peptide
GFSIEEYAIH

SEQ ID NO: 185 - protein, 10 aa, Artificial Sequence
  Description of Artificial Sequence Synthetic peptide
GFTIARSGIH

SEQ ID NO: 186 - protein, 10 aa, Artificial Sequence
  Description of Artificial Sequence Synthetic peptide
GFSITSTGIH

SEQ ID NO: 187 - protein, 10 aa, Artificial Sequence
  Description of Artificial Sequence Synthetic peptide
GFTITGSGIH

SEQ ID NO: 188 - protein, 10 aa, Artificial Sequence
  Description of Artificial Sequence Synthetic peptide
GFSIGGYAIH

SEQ ID NO: 189 - protein, 10 aa, Artificial Sequence
  Description of Artificial Sequence Synthetic peptide
GFSIEGSVIH

SEQ ID NO: 190 - protein, 10 aa, Artificial Sequence
  Description of Artificial Sequence Synthetic peptide
GFTIGGTVIH

SEQ ID NO: 191 - protein, 10 aa, Artificial Sequence
  Description of Artificial Sequence Synthetic peptide
GFSIGRYGIH

SEQ ID NO: 192 - protein, 10 aa, Artificial Sequence
  Description of Artificial Sequence Synthetic peptide
GFSIDKYSIH

SEQ ID NO: 193 - protein, 10 aa, Artificial Sequence
  Description of Artificial Sequence Synthetic peptide
GFSIKTSAIH

SEQ ID NO: 194 - protein, 10 aa, Artificial Sequence
  Description of Artificial Sequence Synthetic peptide
GFSIKTSAIH

SEQ ID NO: 195 - protein, 10 aa, Artificial Sequence
  Description of Artificial Sequence Synthetic peptide
GFSIAGSGIH

SEQ ID NO: 196 - protein, 10 aa, Artificial Sequence
  Description of Artificial Sequence Synthetic peptide
GFSIATSVIH

SEQ ID NO: 197 - protein, 10 aa, Artificial Sequence
  Description of Artificial Sequence Synthetic peptide
GFSIGRSIIH

SEQ ID NO: 198 - protein, 10 aa, Artificial Sequence
  Description of Artificial Sequence Synthetic peptide
GFTIDSNDIH

SEQ ID NO: 199 - protein, 10 aa, Artificial Sequence
  Description of Artificial Sequence Synthetic peptide
GFSIAAYAIH

SEQ ID NO: 200 - protein, 10 aa, Artificial Sequence
  Description of Artificial Sequence Synthetic peptide
GFTIDRNDIH

SEQ ID NO: 201 - protein, 10 aa, Artificial Sequence
  Description of Artificial Sequence Synthetic peptide
GFSIGEYVIH

SEQ ID NO: 202 - protein, 10 aa, Artificial Sequence
  Description of Artificial Sequence Synthetic peptide
GFSIDKSVIP

SEQ ID NO: 203 - protein, 10 aa, Artificial Sequence
  Description of Artificial Sequence Synthetic peptide
GFSITDSVIH

SEQ ID NO: 204 - protein, 10 aa, Artificial Sequence
  Description of Artificial Sequence Synthetic peptide
GFSISNYIIH

SEQ ID NO: 205 - protein, 10 aa, Artificial Sequence
  Description of Artificial Sequence Synthetic peptide
GFSIEASVIH

SEQ ID NO: 206 - protein, 10 aa, Artificial Sequence
  Description of Artificial Sequence Synthetic peptide
GFTIETSTIH

SEQ ID NO: 207 - protein, 10 aa, Artificial Sequence
  Description of Artificial Sequence Synthetic peptide
GFSIAGNTIH

SEQ ID NO: 208 - protein, 10 aa, Artificial Sequence
  Description of Artificial Sequence Synthetic peptide
GFTIADSNIH

SEQ ID NO: 209 - protein, 10 aa, Artificial Sequence
  Description of Artificial Sequence Synthetic peptide
GFTIEEYNIH

SEQ ID NO: 210 - protein, 10 aa, Artificial Sequence
  Description of Artificial Sequence Synthetic peptide
GFSIADTVIH

SEQ ID NO: 211 - protein, 10 aa, Artificial Sequence
  Description of Artificial Sequence Synthetic peptide
GFTIGGTTIH

SEQ ID NO: 212 - protein, 10 aa, Artificial Sequence
  Description of Artificial Sequence Synthetic peptide
GFTIGNSTIH

SEQ ID NO: 213 - protein, 10 aa, Artificial Sequence
  Description of Artificial Sequence Synthetic peptide
GFSIDKSVIH

SEQ ID NO: 214 - protein, 10 aa, Artificial Sequence
  Description of Artificial Sequence Synthetic peptide
GFSIDEYVIH

SEQ ID NO: 215 - protein, 10 aa, Artificial Sequence
  Description of Artificial Sequence Synthetic peptide
GFSIDGSSIH

SEQ ID NO: 216 - protein, 10 aa, Artificial Sequence
  Description of Artificial Sequence Synthetic peptide
GFSIDRYVIH

SEQ ID NO: 217 - protein, 10 aa, Artificial Sequence
  Description of Artificial Sequence Synthetic peptide
GFTIRTNAIH

SEQ ID NO: 218 - protein, 10 aa, Artificial Sequence
  Description of Artificial Sequence Synthetic peptide
GFTIRTNAIH

SEQ ID NO: 219 - protein, 10 aa, Artificial Sequence
  Description of Artificial Sequence Synthetic peptide
GFSIGNSAIH

SEQ ID NO: 220 - protein, 10 aa, Artificial Sequence
  Description of Artificial Sequence Synthetic peptide
GFSITNYVIH

SEQ ID NO: 221 - protein, 10 aa, Artificial Sequence
  Description of Artificial Sequence Synthetic peptide
GFTIDETTIH

SEQ ID NO: 222 - protein, 10 aa, Artificial Sequence
  Description of Artificial Sequence Synthetic peptide
GFTIDETTIH

SEQ ID NO: 223 - protein, 10 aa, Artificial Sequence
  Description of Artificial Sequence Synthetic peptide
GFSINNYVIH

SEQ ID NO: 224 - protein, 10 aa, Artificial Sequence
  Description of Artificial Sequence Synthetic peptide
GFSIDGYAIH

SEQ ID NO: 225 - protein, 10 aa, Artificial Sequence
  Description of Artificial Sequence Synthetic peptide
GFSIGRYSIH

SEQ ID NO: 226 - protein, 10 aa, Artificial Sequence
  Description of Artificial Sequence Synthetic peptide
GFSIDKTVIH

SEQ ID NO: 227 - protein, 10 aa, Artificial Sequence
  Description of Artificial Sequence Synthetic peptide
GFSIDKTVIH

SEQ ID NO: 228 - protein, 10 aa, Artificial Sequence
  Description of Artificial Sequence Synthetic peptide
GFTIANTTIH

SEQ ID NO: 229 - protein, 10 aa, Artificial Sequence
  Description of Artificial Sequence Synthetic peptide
GFSIAGSIIH

SEQ ID NO: 230 - protein, 14 aa, Artificial Sequence
  Description of Artificial Sequence Synthetic peptide
VATIYPTYGYTYYA

SEQ ID NO: 231 - protein, 14 aa, Artificial Sequence
  Description of Artificial Sequence Synthetic peptide
VALIAPSAGATNYA

SEQ ID NO: 232 - protein, 14 aa, Artificial Sequence
  Description of Artificial Sequence Synthetic peptide
VATIFPTDGYTNYA

SEQ ID NO: 233 - protein, 14 aa, Artificial Sequence
  Description of Artificial Sequence Synthetic peptide
VASIAPYDGDTAYA

SEQ ID NO: 234 - protein, 14 aa, Artificial Sequence
  Description of Artificial Sequence Synthetic peptide
VAMISPSTGTTTAD

SEQ ID NO: 235 - protein, 14 aa, Artificial Sequence
  Description of Artificial Sequence Synthetic peptide
VARIYPXXRPXTRY

SEQ ID NO: 236 - protein, 14 aa, Artificial Sequence
  Description of Artificial Sequence Synthetic peptide
VAGIAPYNGDTTYA

SEQ ID NO: 237 - protein, 14 aa, Artificial Sequence
  Description of Artificial Sequence Synthetic peptide
VASIVPAYADTYYA

SEQ ID NO: 238 - protein, 14 aa, Artificial Sequence
  Description of Artificial Sequence Synthetic peptide
VARIAPHSGDTTYA

SEQ ID NO: 239 - protein, 14 aa, Artificial Sequence
  Description of Artificial Sequence Synthetic peptide
VAGIVPATGNTYYA

SEQ ID NO: 240 - protein, 14 aa, Artificial Sequence
  Description of Artificial Sequence Synthetic peptide
VAGIIPYTGSTSYA

SEQ ID NO: 241 - protein, 14 aa, Artificial Sequence
  Description of Artificial Sequence Synthetic peptide
VAGIAPYNGTTDYA

SEQ ID NO: 242 - protein, 14 aa, Artificial Sequence
  Description of Artificial Sequence Synthetic peptide
VARIFPHSGATTYA

SEQ ID NO: 243 - protein, 14 aa, Artificial Sequence
  Description of Artificial Sequence Synthetic peptide
VALIYPHSGATSYA

SEQ ID NO: 244 - protein, 14 aa, Artificial Sequence
  Description of Artificial Sequence Synthetic peptide
VAGIVPAAGNTDYA

SEQ ID NO: 245 - protein, 14 aa, Artificial Sequence
  Description of Artificial Sequence Synthetic peptide
VATIAPYNGNTTYA

SEQ ID NO: 246 - protein, 14 aa, Artificial Sequence
  Description of Artificial Sequence Synthetic peptide
VAWIIPYTGSTSYA

SEQ ID NO: 247 - protein, 14 aa, Artificial Sequence
  Description of Artificial Sequence Synthetic peptide
FARIYPTYGATDYA

SEQ ID NO: 248 - protein, 14 aa, Artificial Sequence
  Description of Artificial Sequence Synthetic peptide
VARIFPSAGYTNYA

SEQ ID NO: 249 - protein, 14 aa, Artificial Sequence
  Description of Artificial Sequence Synthetic peptide
VASIIPYYGTTAYA

SEQ ID NO: 250 - protein, 14 aa, Artificial Sequence
  Description of Artificial Sequence Synthetic peptide
VATIIPYTGNTYYA

SEQ ID NO: 251 - protein, 14 aa, Artificial Sequence
  Description of Artificial Sequence Synthetic peptide
VAGIVPSYGNTYYA

SEQ ID NO: 252 - protein, 14 aa, Artificial Sequence
  Description of Artificial Sequence Synthetic peptide
VAGIVPHAGATYYA

SEQ ID NO: 253 - protein, 14 aa, Artificial Sequence
  Description of Artificial Sequence Synthetic peptide
VAMIYPNYGATTYA

SEQ ID NO: 254 - protein, 14 aa, Artificial Sequence
  Description of Artificial Sequence Synthetic peptide
VAWIVPAYGSTSYA

SEQ ID NO: 255 - protein, 14 aa, Artificial Sequence
  Description of Artificial Sequence Synthetic peptide
VAGIYPHAGSTTYA

SEQ ID NO: 256 - protein, 14 aa, Artificial Sequence
  Description of Artificial Sequence Synthetic peptide
VAWIDPAAGATAYA

SEQ ID NO: 257 - protein, 14 aa, Artificial Sequence
  Description of Artificial Sequence Synthetic peptide
VARINPNSGSTYYA

SEQ ID NO: 258 - protein, 14 aa, Artificial Sequence
  Description of Artificial Sequence Synthetic peptide
VAMIIPYTGDTSYA

SEQ ID NO: 259 - protein, 14 aa, Artificial Sequence
  Description of Artificial Sequence Synthetic peptide
VAMIYPDNGYTNYA

SEQ ID NO: 260 - protein, 14 aa, Artificial Sequence
  Description of Artificial Sequence Synthetic peptide
VASIAPSDGATSYA

SEQ ID NO: 261 - protein, 14 aa, Artificial Sequence
  Description of Artificial Sequence Synthetic peptide
VARIIPYTGATTYA

SEQ ID NO: 262 - protein, 14 aa, Artificial Sequence
  Description of Artificial Sequence Synthetic peptide
VAGIVPTAGYTYYA

SEQ ID NO: 263 - protein, 14 aa, Artificial Sequence
  Description of Artificial Sequence Synthetic peptide
FAGIVPTAGYTYYA

SEQ ID NO: 264 - protein, 14 aa, Artificial Sequence
  Description of Artificial Sequence Synthetic peptide
VAGIAPASGSTNYA

SEQ ID NO: 265 - protein, 14 aa, Artificial Sequence
  Description of Artificial Sequence Synthetic peptide
VARIIPNNGSTAYA

SEQ ID NO: 266 - protein, 14 aa, Artificial Sequence
  Description of Artificial Sequence Synthetic peptide
FVVISPYSGYTNYA

SEQ ID NO: 267 - protein, 14 aa, Artificial Sequence
  Description of Artificial Sequence Synthetic peptide
VARITPYTGATYYA

SEQ ID NO: 268 - protein, 14 aa, Artificial Sequence
  Description of Artificial Sequence Synthetic peptide
VATIIPANGDTNYA

SEQ ID NO: 269 - protein, 14 aa, Artificial Sequence
  Description of Artificial Sequence Synthetic peptide
VARITPYTGATDYA

SEQ ID NO: 270 - protein, 14 aa, Artificial Sequence
  Description of Artificial Sequence Synthetic peptide
VARIIPYDGSTAYA

SEQ ID NO: 271 - protein, 14 aa, Artificial Sequence
  Description of Artificial Sequence Synthetic peptide
VARIIPAYGTTYYA

SEQ ID NO: 272 - protein, 14 aa, Artificial Sequence
  Description of Artificial Sequence Synthetic peptide
VARIDPPTGATYYA

SEQ ID NO: 273 - protein, 14 aa, Artificial Sequence
  Description of Artificial Sequence Synthetic peptide
VARIDPTNGNTYYA

SEQ ID NO: 274 - protein, 14 aa, Artificial Sequence
  Description of Artificial Sequence Synthetic peptide
VARIIPYTGNTNYA

SEQ ID NO: 275 - protein, 14 aa, Artificial Sequence
  Description of Artificial Sequence Synthetic peptide
VAGIIPYTGTTDYA

SEQ ID NO: 276 - protein, 14 aa, Artificial Sequence
  Description of Artificial Sequence Synthetic peptide
VAGITPATGYTNYA

SEQ ID NO: 277 - protein, 14 aa, Artificial Sequence
  Description of Artificial Sequence Synthetic peptide
VARIFPHTGDTYYA

SEQ ID NO: 278 - protein, 14 aa, Artificial Sequence
  Description of Artificial Sequence Synthetic peptide
FARIVPYTGYTYYA

SEQ ID NO: 279 - protein, 14 aa, Artificial Sequence
  Description of Artificial Sequence Synthetic peptide
VARIYPYAGSTNYA

SEQ ID NO: 280 - protein, 14 aa, Artificial Sequence
  Description of Artificial Sequence Synthetic peptide
VASINPHSGSTAYA

SEQ ID NO: 281 - protein, 14 aa, Artificial Sequence
  Description of Artificial Sequence Synthetic peptide
VAGIIPASGSTAYA

SEQ ID NO: 282 - protein, 14 aa, Artificial Sequence
  Description of Artificial Sequence Synthetic peptide
VARIIPAYGTTYYA

SEQ ID NO: 283 - protein, 14 aa, Artificial Sequence
  Description of Artificial Sequence Synthetic peptide
VARIDPYSGATYYA

SEQ ID NO: 284 - protein, 14 aa, Artificial Sequence
  Description of Artificial Sequence Synthetic peptide
VARITPYSGNTTYA

SEQ ID NO: 285 - protein, 14 aa, Artificial Sequence
  Description of Artificial Sequence Synthetic peptide
VARIAPYSGDTNYA

SEQ ID NO: 286 - protein, 14 aa, Artificial Sequence
  Description of Artificial Sequence Synthetic peptide
VARINPYSGYTTYA

SEQ ID NO: 287 - protein, 14 aa, Artificial Sequence
  Description of Artificial Sequence Synthetic peptide
FARINPYSGYTTYA

SEQ ID NO: 288 - protein, 14 aa, Artificial Sequence
  Description of Artificial Sequence Synthetic peptide
VARINPHTGATTYA

SEQ ID NO: 289 - protein, 14 aa, Artificial Sequence
  Description of Artificial Sequence Synthetic peptide
VAGITPHSGYTAYA

SEQ ID NO: 290 - protein, 14 aa, Artificial Sequence
  Description of Artificial Sequence Synthetic peptide
VAMIAPAYGATTYA

SEQ ID NO: 291 - protein, 14 aa, Artificial Sequence
  Description of Artificial Sequence Synthetic peptide
VAMIAPAYGATTYA

SEQ ID NO: 292 - protein, 14 aa, Artificial Sequence
  Description of Artificial Sequence Synthetic peptide
VAGIIPYTGNTYYA

SEQ ID NO: 293 - protein, 14 aa, Artificial Sequence
  Description of Artificial Sequence Synthetic peptide
VARIYPASGATNYA

SEQ ID NO: 294 - protein, 14 aa, Artificial Sequence
  Description of Artificial Sequence Synthetic peptide
VARIDPDAGATDYA

SEQ ID NO: 295 - protein, 14 aa, Artificial Sequence
  Description of Artificial Sequence Synthetic peptide
VALISPYTGTTTYA

SEQ ID NO: 296 - protein, 14 aa, Artificial Sequence
  Description of Artificial Sequence Synthetic peptide
FALISPYTGTTTYA

SEQ ID NO: 297 - protein, 14 aa, Artificial Sequence
  Description of Artificial Sequence Synthetic peptide
FAGINPASGDTTYA

SEQ ID NO: 298 - protein, 14 aa, Artificial Sequence
  Description of Artificial Sequence Synthetic peptide
VAMIAPTSGNTAYA

SEQ ID NO: 299 - protein, 9 aa, Artificial Sequence
  Description of Artificial Sequence Synthetic peptide
SREGKYAMD

SEQ ID NO: 300 - protein, 9 aa, Artificial Sequence
  Description of Artificial Sequence Synthetic peptide
SRSAWYAMD

SEQ ID NO: 301 - protein, 9 aa, Artificial Sequence
  Description of Artificial Sequence Synthetic peptide
SRKNRYAMD

SEQ ID NO: 302 - protein, 10 aa, Artificial Sequence
  Description of Artificial Sequence Synthetic peptide
SRGGWFYAMD

SEQ ID NO: 303 - protein, 10 aa, Artificial Sequence
  Description of Artificial Sequence Synthetic peptide
RAATRSYAMD

SEQ ID NO: 304 - protein, 10 aa, Artificial Sequence
  Description of Artificial Sequence Synthetic peptide
CSRAGIYAMD

SEQ ID NO: 305 - protein, 11 aa, Artificial Sequence
  Description of Artificial Sequence Synthetic peptide
SRAYSRQYAMD

SEQ ID NO: 306 - protein, 11 aa, Artificial Sequence
  Description of Artificial Sequence Synthetic peptide
SRSSRSMYTMD

SEQ ID NO: 307 - protein, 11 aa, Artificial Sequence
  Description of Artificial Sequence Synthetic peptide
SRAYYREYAMD

SEQ ID NO: 308 - protein, 11 aa, Artificial Sequence
  Description of Artificial Sequence Synthetic peptide
SRGRYAMYAMD

SEQ ID NO: 309 - protein, 11 aa, Artificial Sequence
  Description of Artificial Sequence Synthetic peptide
SRGSRSEYAMD

SEQ ID NO: 310 - protein, 11 aa, Artificial Sequence
  Description of Artificial Sequence Synthetic peptide
SRAWYAQYAMD

SEQ ID NO: 311 - protein, 11 aa, Artificial Sequence
  Description of Artificial Sequence Synthetic peptide
SRSWYAEYAMD

SEQ ID NO: 312 - protein, 11 aa, Artificial Sequence
  Description of Artificial Sequence Synthetic peptide
SRSWKAEYAMD

SEQ ID NO: 313 - protein, 11 aa, Artificial Sequence
  Description of Artificial Sequence Synthetic peptide
SRSWWEHYAMD

SEQ ID NO: 314 - protein, 11 aa, Artificial Sequence
  Description of Artificial Sequence Synthetic peptide
SRARYSMYAMD

SEQ ID NO: 315 - protein, 11 aa, Artificial Sequence
  Description of Artificial Sequence Synthetic peptide
SRGSRSEYAMD

SEQ ID NO: 316 - protein, 11 aa, Artificial Sequence
  Description of Artificial Sequence Synthetic peptide
SRDWWTLYAMD

SEQ ID NO: 317 - protein, 12 aa, Artificial Sequence
  Description of Artificial Sequence Synthetic peptide
SRWSGSRRYAMD

SEQ ID NO: 318 - protein, 12 aa, Artificial Sequence
  Description of Artificial Sequence Synthetic peptide
SRDGNSGHYAMD

SEQ ID NO: 319 - protein, 12 aa, Artificial Sequence
  Description of Artificial Sequence Synthetic peptide
SRTYGWSGYAMD

SEQ ID NO: 320 - protein, 12 aa, Artificial Sequence
  Description of Artificial Sequence Synthetic peptide
SRNYSGYFYAMD

SEQ ID NO: 321 - protein, 12 aa, Artificial Sequence
  Description of Artificial Sequence Synthetic peptide
SRGYSYTFYAMD

SEQ ID NO: 322 - protein, 12 aa, Artificial Sequence
  Description of Artificial Sequence Synthetic peptide
SRNYAGALYAMD

SEQ ID NO: 323 - protein, 12 aa, Artificial Sequence
  Description of Artificial Sequence Synthetic peptide
SRSATGEVYAMD

SEQ ID NO: 324 - protein, 13 aa, Artificial Sequence
  Description of Artificial Sequence Synthetic peptide
SRGSYKAWFYAMD

SEQ ID NO: 325 - protein, 13 aa, Artificial Sequence
  Description of Artificial Sequence Synthetic peptide
SREGSGWATYAMD

SEQ ID NO: 326 - protein, 14 aa, Artificial Sequence
  Description of Artificial Sequence Synthetic peptide
SRGAGYSKSAYAMD

SEQ ID NO: 327 - protein, 14 aa, Artificial Sequence
  Description of Artificial Sequence Synthetic peptide
SRGEATWRRAYAMD

SEQ ID NO: 328 - protein, 14 aa, Artificial Sequence
  Description of Artificial Sequence Synthetic peptide
SRNDYSGTALYAMD

SEQ ID NO: 329 - protein, 14 aa, Artificial Sequence
  Description of Artificial Sequence Synthetic peptide
SRGAGYSYTLYAMD

SEQ ID NO: 330 - protein, 14 aa, Artificial Sequence
  Description of Artificial Sequence Synthetic peptide
SRGGTSWSRLYAMD

SEQ ID NO: 331 - protein, 14 aa, Artificial Sequence
  Description of Artificial Sequence Synthetic peptide
SRGDGTWGKLYAMD

SEQ ID NO: 332 - protein, 14 aa, Artificial Sequence
  Description of Artificial Sequence Synthetic peptide
SRGDGTWGKLYAMD

SEQ ID NO: 333 - protein, 14 aa, Artificial Sequence
  Description of Artificial Sequence Synthetic peptide
SRRAGYSYTLYAMD

SEQ ID NO: 334 - protein, 14 aa, Artificial Sequence
  Description of Artificial Sequence Synthetic peptide
SRAAARRSYMYAMD

SEQ ID NO: 335 - protein, 14 aa, Artificial Sequence
  Description of Artificial Sequence Synthetic peptide
SRGGTSYRSMYAMD

SEQ ID NO: 336 - protein, 14 aa, Artificial Sequence
  Description of Artificial Sequence Synthetic peptide
SRNRNSWAWRYAMD

SEQ ID NO: 337 - protein, 14 aa, Artificial Sequence
  Description of Artificial Sequence Synthetic peptide
SRGAGRSYTRYAMD

SEQ ID NO: 338 - protein, 14 aa, Artificial Sequence
  Description of Artificial Sequence Synthetic peptide
SRNRNTWTRRYAMD

SEQ ID NO: 339 - protein, 14 aa, Artificial Sequence
  Description of Artificial Sequence Synthetic peptide
SRGWARWSRRYAMD

SEQ ID NO: 340 - protein, 14 aa, Artificial Sequence
  Description of Artificial Sequence Synthetic peptide
SRGGNSYTTRYAMD

SEQ ID NO: 341 - protein, 14 aa, Artificial Sequence
  Description of Artificial Sequence Synthetic peptide
SRSSNSWTRRYAMD

SEQ ID NO: 342 - protein, 14 aa, Artificial Sequence
  Description of Artificial Sequence Synthetic peptide
SRAWATWGRRYAMD

SEQ ID NO: 343 - protein, 14 aa, Artificial Sequence
  Description of Artificial Sequence Synthetic peptide
SRSSASWKSRYAMD

SEQ ID NO: 344 - protein, 14 aa, Artificial Sequence
  Description of Artificial Sequence Synthetic peptide
SRGGASWTRRYAMD

SEQ ID NO: 345 - protein, 14 aa, Artificial Sequence
  Description of Artificial Sequence Synthetic peptide
SRGGGRYAWRYAMD

SEQ ID NO: 346 - protein, 14 aa, Artificial Sequence
  Description of Artificial Sequence Synthetic peptide
SRSAGTWSRRYAMD

SEQ ID NO: 347 - protein, 14 aa, Artificial Sequence
  Description of Artificial Sequence Synthetic peptide
SRSTASRSSRYAMD

SEQ ID NO: 348 - protein, 14 aa, Artificial Sequence
  Description of Artificial Sequence Synthetic peptide
SRSANSWSTRYAMD

SEQ ID NO: 349 - protein, 14 aa, Artificial Sequence
  Description of Artificial Sequence Synthetic peptide
SRGAGYSYTRYAMD

SEQ ID NO: 350 - protein, 14 aa, Artificial Sequence
  Description of Artificial Sequence Synthetic peptide
SRGGGRWSRRYAMD

SEQ ID NO: 351 - protein, 14 aa, Artificial Sequence
  Description of Artificial Sequence Synthetic peptide
SRGGNSYTTRYAMD

SEQ ID NO: 352 - protein, 14 aa, Artificial Sequence
  Description of Artificial Sequence Synthetic peptide
SRATGTWSSRYAMD

SEQ ID NO: 353 - protein, 14 aa, Artificial Sequence
  Description of Artificial Sequence Synthetic peptide
SRGSAYYSSTYAMD

SEQ ID NO: 354 - protein, 14 aa, Artificial Sequence
  Description of Artificial Sequence Synthetic peptide
SRGANSRSRVYAMD

SEQ ID NO: 355 - protein, 14 aa, Artificial Sequence
  Description of Artificial Sequence Synthetic peptide
SRWGNTETAVYAMD

SEQ ID NO: 356 - protein, 14 aa, Artificial Sequence
  Description of Artificial Sequence Synthetic peptide
SRWGNTETAVYAMD

SEQ ID NO: 357 - protein, 14 aa, Artificial Sequence
  Description of Artificial Sequence Synthetic peptide
SRWGADSWAVYAMD

SEQ ID NO: 358 - protein, 15 aa, Artificial Sequence
  Description of Artificial Sequence Synthetic peptide
SRGRKASYRARYAMD

SEQ ID NO: 359 - protein, 15 aa, Artificial Sequence
  Description of Artificial Sequence Synthetic peptide
SRGKSWRACEYYAMD

SEQ ID NO: 360 - protein, 15 aa, Artificial Sequence
  Description of Artificial Sequence Synthetic peptide
SRGKSWRAWEYYAMD

SEQ ID NO: 361 - protein, 15 aa, Artificial Sequence
  Description of Artificial Sequence Synthetic peptide
SRGRAATYTGQYAMD

SEQ ID NO: 362 - protein, 15 aa, Artificial Sequence
  Description of Artificial Sequence Synthetic peptide
SRSGATYRGSRYAMD

SEQ ID NO: 363 - protein, 16 aa, Artificial Sequence
  Description of Artificial Sequence Synthetic peptide
SRGGTKARYSELYAMD

SEQ ID NO: 364 - protein, 16 aa, Artificial Sequence
  Description of Artificial Sequence Synthetic peptide
SRGGWSARGYSSYAMD

SEQ ID NO: 365 - protein, 16 aa, Artificial Sequence
  Description of Artificial Sequence Synthetic peptide
SRGGWSAMGYSSYAMD

SEQ ID NO: 366 - protein, 18 aa, Artificial Sequence
  Description of Artificial Sequence Synthetic peptide
SRERYWTSGTTYGSYAMD

SEQ ID NO: 367 - protein, 19 aa, Artificial Sequence
  Description of Artificial Sequence Synthetic peptide
SRSWSSWGWGSSTGRYAMD

SEQ ID NO: 368 - protein, 10 aa, Artificial Sequence
  Description of Artificial Sequence Synthetic peptide
GFTISATAIH

SEQ ID NO: 369 - protein, 10 aa, Artificial Sequence
  Description of Artificial Sequence Synthetic peptide
GFTISDTAIH

SEQ ID NO: 370 - protein, 10 aa, Artificial Sequence
  Description of Artificial Sequence Synthetic peptide
GFTISATAIH

SEQ ID NO: 371 - protein, 10 aa, Artificial Sequence
  Description of Artificial Sequence Synthetic peptide
GFTISDTAIH

SEQ ID NO: 372 - protein, 10 aa, Artificial Sequence
  Description of Artificial Sequence Synthetic peptide
GFTINATAIH

SEQ ID NO: 373 - protein, 10 aa, Artificial Sequence
  Description of Artificial Sequence Synthetic peptide
GFTINSTAIH

SEQ ID NO: 374 - protein, 10 aa, Artificial Sequence
  Description of Artificial Sequence Synthetic peptide
GFTISATAIH

SEQ ID NO: 375 - protein, 10 aa, Artificial Sequence
  Description of Artificial Sequence Synthetic peptide
GFTITSTAIH

SEQ ID NO: 376 - protein, 10 aa, Artificial Sequence
  Description of Artificial Sequence Synthetic peptide
GFTINATAIH

SEQ ID NO: 377 - protein, 10 aa, Artificial Sequence
  Description of Artificial Sequence Synthetic peptide
GFTISDTAIH

SEQ ID NO: 378 - protein, 10 aa, Artificial Sequence
  Description of Artificial Sequence Synthetic peptide
GFTINSTAIH

SEQ ID NO: 379 - protein, 10 aa, Artificial Sequence
  Description of Artificial Sequence Synthetic peptide
GFTITSTAIH

SEQ ID NO: 380 - protein, 10 aa, Artificial Sequence
  Description of Artificial Sequence Synthetic peptide
GFTITRTSIH

SEQ ID NO: 381 - protein, 10 aa, Artificial Sequence
  Description of Artificial Sequence Synthetic peptide
GFTINNTWIH

SEQ ID NO: 382 - protein, 10 aa, Artificial Sequence
  Description of Artificial Sequence Synthetic peptide
GFTISDTAIH

SEQ ID NO: 383 - protein, 10 aa, Artificial Sequence
  Description of Artificial Sequence Synthetic peptide
GFTITNSGIH

SEQ ID NO: 384 - protein, 10 aa, Artificial Sequence
  Description of Artificial Sequence Synthetic peptide
GFTINATAIH

SEQ ID NO: 385 - protein, 10 aa, Artificial Sequence
  Description of Artificial Sequence Synthetic peptide
GFTISGSYIH

SEQ ID NO: 386 - protein, 10 aa, Artificial Sequence
  Description of Artificial Sequence Synthetic peptide
GFTITDSWIH

SEQ ID NO: 387 - protein, 10 aa, Artificial Sequence
  Description of Artificial Sequence Synthetic peptide
GFTINATAIH

SEQ ID NO: 388 - protein, 10 aa, Artificial Sequence
  Description of Artificial Sequence Synthetic peptide
GFTITNSGIH

SEQ ID NO: 389 - protein, 10 aa, Artificial Sequence
  Description of Artificial Sequence Synthetic peptide
GFTINNSDIH

SEQ ID NO: 390 - protein, 10 aa, Artificial Sequence
  Description of Artificial Sequence Synthetic peptide
GFTITGTGIH

SEQ ID NO: 391 - protein, 10 aa, Artificial Sequence
  Description of Artificial Sequence Synthetic peptide
GFTITSTSIH

SEQ ID NO: 392 - protein, 10 aa, Artificial Sequence
  Description of Artificial Sequence Synthetic peptide
GFTITNSGIH

SEQ ID NO: 393 - protein, 10 aa, Artificial Sequence
  Description of Artificial Sequence Synthetic peptide
GFTITETSIH

SEQ ID NO: 394 - protein, 10 aa, Artificial Sequence
  Description of Artificial Sequence Synthetic peptide
GFTISSTSIH

SEQ ID NO: 395 - protein, 10 aa, Artificial Sequence
  Description of Artificial Sequence Synthetic peptide
GFTITDTSIH

SEQ ID NO: 396 - protein, 10 aa, Artificial Sequence
  Description of Artificial Sequence Synthetic peptide
GFTINATYIH

SEQ ID NO: 397 - protein, 10 aa, Artificial Sequence
  Description of Artificial Sequence Synthetic peptide
GFTITNSGIH

SEQ ID NO: 398 - protein, 10 aa, Artificial Sequence
  Description of Artificial Sequence Synthetic peptide
GFTITGTAIH

SEQ ID NO: 399 - protein, 10 aa, Artificial Sequence
  Description of Artificial Sequence Synthetic peptide
GFTINSTAIH

SEQ ID NO: 400 - protein, 10 aa, Artificial Sequence
  Description of Artificial Sequence Synthetic peptide
GFTINTSWIH

SEQ ID NO: 401 - protein, 14 aa, Artificial Sequence
  Description of Artificial Sequence Synthetic peptide
VARITPSDGTTDYA

SEQ ID NO: 402 - protein, 14 aa, Artificial Sequence
  Description of Artificial Sequence Synthetic peptide
VASISPSSGATYYA

SEQ ID NO: 403 - protein, 14 aa, Artificial Sequence
  Description of Artificial Sequence Synthetic peptide
VARITPSDGTTDYA

SEQ ID NO: 404 - protein, 14 aa, Artificial Sequence
  Description of Artificial Sequence Synthetic peptide
VASISPSSGATYYA

SEQ ID NO: 405 - protein, 14 aa, Artificial Sequence
  Description of Artificial Sequence Synthetic peptide
VARINPAGGNTYYA

SEQ ID NO: 406 - protein, 14 aa, Artificial Sequence
  Description of Artificial Sequence Synthetic peptide
VAFIYPSDGATDYA

SEQ ID NO: 407 - protein, 14 aa, Artificial Sequence
  Description of Artificial Sequence Synthetic peptide
VARITPSDGTTDYA

SEQ ID NO: 408 - protein, 14 aa, Artificial Sequence
  Description of Artificial Sequence Synthetic peptide
VAYITPYSGYTYYA

SEQ ID NO: 409 - protein, 14 aa, Artificial Sequence
  Description of Artificial Sequence Synthetic peptide
VAFISPTGGATNYA

SEQ ID NO: 410 - protein, 14 aa, Artificial Sequence
  Description of Artificial Sequence Synthetic peptide
VASISPSSGATYYA

SEQ ID NO: 411 - protein, 14 aa, Artificial Sequence
  Description of Artificial Sequence Synthetic peptide
VARISPASGATYYA

SEQ ID NO: 412 - protein, 14 aa, Artificial Sequence
  Description of Artificial Sequence Synthetic peptide
VAYISPYSGYTYYA

SEQ ID NO: 413 - protein, 14 aa, Artificial Sequence
  Description of Artificial Sequence Synthetic peptide
VAVINPTSGSTDYA

SEQ ID NO: 414 - protein, 14 aa, Artificial Sequence
  Description of Artificial Sequence Synthetic peptide
VAFISPASGATNYA

SEQ ID NO: 415 - protein, 14 aa, Artificial Sequence
  Description of Artificial Sequence Synthetic peptide
VARINPSSGSTYYA

SEQ ID NO: 416 - protein, 14 aa, Artificial Sequence
  Description of Artificial Sequence Synthetic peptide
VADIYPHSGSTDYA

SEQ ID NO: 417 - protein, 14 aa, Artificial Sequence
  Description of Artificial Sequence Synthetic peptide
VAFISPTGGATNYA

SEQ ID NO: 418 - protein, 14 aa, Artificial Sequence
  Description of Artificial Sequence Synthetic peptide
VARISPSGGYTYYA

SEQ ID NO: 419 - protein, 14 aa, Artificial Sequence
  Description of Artificial Sequence Synthetic peptide
VAWISPSSGSTYYA

SEQ ID NO: 420 - protein, 14 aa, Artificial Sequence
  Description of Artificial Sequence Synthetic peptide
VAFISPTGGATNYA

SEQ ID NO: 421 - protein, 14 aa, Artificial Sequence
  Description of Artificial Sequence Synthetic peptide
VADIYPHSGSTDYA

SEQ ID NO: 422 - protein, 14 aa, Artificial Sequence
  Description of Artificial Sequence Synthetic peptide
VAWISPHGGYTDYA

SEQ ID NO: 423 - protein, 14 aa, Artificial Sequence
  Description of Artificial Sequence Synthetic peptide
VAAINPSDGSTDYA

SEQ ID NO: 424 - protein, 14 aa, Artificial Sequence
  Description of Artificial Sequence Synthetic peptide
VAFISPTSGYTYYA

SEQ ID NO: 425 - protein, 14 aa, Artificial Sequence
  Description of Artificial Sequence Synthetic peptide
VADIYPHSGSTDYA

SEQ ID NO: 426 - protein, 14 aa, Artificial Sequence
  Description of Artificial Sequence Synthetic peptide
VADISPNDGNTDYA

SEQ ID NO: 427 - protein, 14 aa, Artificial Sequence
  Description of Artificial Sequence Synthetic peptide
VARIYPSDGDTNYA

SEQ ID NO: 428 - protein, 14 aa, Artificial Sequence
  Description of Artificial Sequence Synthetic peptide
VAFINPNGGNTYYA

SEQ ID NO: 429 - protein, 14 aa, Artificial Sequence
  Description of Artificial Sequence Synthetic peptide
VARISPSNGNTNYA

SEQ ID NO: 430 - protein, 14 aa, Artificial Sequence
  Description of Artificial Sequence Synthetic peptide
VAGIYPYNGDTYYA

SEQ ID NO: 431 - protein, 14 aa, Artificial Sequence
  Description of Artificial Sequence Synthetic peptide
VARISPNGGSTNYA

SEQ ID NO: 432 - protein, 14 aa, Artificial Sequence
  Description of Artificial Sequence Synthetic peptide
VAFISPSNGSTYYA

SEQ ID NO: 433 - protein, 14 aa, Artificial Sequence
  Description of Artificial Sequence Synthetic peptide
VAWISPNGGYTNYA

SEQ ID NO: 434 - protein, 11 aa, Artificial Sequence
  Description of Artificial Sequence Synthetic peptide
ARQLTLSGGMD

SEQ ID NO: 435 - protein, 11 aa, Artificial Sequence
  Description of Artificial Sequence Synthetic peptide
ARLLSRSGAMD

SEQ ID NO: 436 - protein, 11 aa, Artificial Sequence
  Description of Artificial Sequence Synthetic peptide
ARQLTLSGVMD

SEQ ID NO: 437 - protein, 11 aa, Artificial Sequence
  Description of Artificial Sequence Synthetic peptide
ARLLSRSGAMD

SEQ ID NO: 438 - protein, 11 aa, Artificial Sequence
  Description of Artificial Sequence Synthetic peptide
ARSLSLSGAMD

SEQ ID NO: 439 - protein, 11 aa, Artificial Sequence
  Description of Artificial Sequence Synthetic peptide
ARLLTRSGAMD

SEQ ID NO: 440 - protein, 11 aa, Artificial Sequence
  Description of Artificial Sequence Synthetic peptide
ARQLTLSGVMD

SEQ ID NO: 441 - protein, 11 aa, Artificial Sequence
  Description of Artificial Sequence Synthetic peptide
ARSFSWRGVMD

SEQ ID NO: 442 - protein, 11 aa, Artificial Sequence
  Description of Artificial Sequence Synthetic peptide
ARALTISGVMD

SEQ ID NO: 443 - protein, 11 aa, Artificial Sequence
  Description of Artificial Sequence Synthetic peptide
ARLLSRSGAMD

SEQ ID NO: 444 - protein, 11 aa, Artificial Sequence
  Description of Artificial Sequence Synthetic peptide
ARAATLRGVMD

SEQ ID NO: 445 - protein, 11 aa, Artificial Sequence
  Description of Artificial Sequence Synthetic peptide
ARSFSRGGVMD

SEQ ID NO: 446 - protein, 11 aa, Artificial Sequence
  Description of Artificial Sequence Synthetic peptide
ARLLGRWSGMD

SEQ ID NO: 447 - protein, 11 aa, Artificial Sequence
  Description of Artificial Sequence Synthetic peptide
ARLFSLSGAMD

SEQ ID NO: 448 - protein, 11 aa, Artificial Sequence
  Description of Artificial Sequence Synthetic peptide
ARSLSRWYVMD

SEQ ID NO: 449 - protein, 11 aa, Artificial Sequence
  Description of Artificial Sequence Synthetic peptide
ARGRVAEYVMD

SEQ ID NO: 450 - protein, 11 aa, Artificial Sequence
  Description of Artificial Sequence Synthetic peptide
ARALTISGVMD

SEQ ID NO: 451 - protein, 11 aa, Artificial Sequence
  Description of Artificial Sequence Synthetic peptide
ARGFTYHGVMD

SEQ ID NO: 452 - protein, 11 aa, Artificial Sequence
  Description of Artificial Sequence Synthetic peptide
ARLLALSGAMD

SEQ ID NO: 453 - protein, 11 aa, Artificial Sequence
  Description of Artificial Sequence Synthetic peptide
ARALTISGVMD

SEQ ID NO: 454 - protein, 11 aa, Artificial Sequence
  Description of Artificial Sequence Synthetic peptide
ARGRVAEYVMD

SEQ ID NO: 455 - protein, 11 aa, Artificial Sequence
  Description of Artificial Sequence Synthetic peptide
ARRVSRSGAMD

SEQ ID NO: 456 - protein, 11 aa, Artificial Sequence
  Description of Artificial Sequence Synthetic peptide
ARLLSLSGAMD

SEQ ID NO: 457 - protein, 11 aa, Artificial Sequence
  Description of Artificial Sequence Synthetic peptide
ARAATRSYAMD

SEQ ID NO: 458 - protein, 11 aa, Artificial Sequence
  Description of Artificial Sequence Synthetic peptide
ARGRVVEYVMD

SEQ ID NO: 459 - protein, 11 aa, Artificial Sequence
  Description of Artificial Sequence Synthetic peptide
ARKLSVSGAMD

SEQ ID NO: 460 - protein, 11 aa, Artificial Sequence
  Description of Artificial Sequence Synthetic peptide
ARALTVRGAMD

SEQ ID NO: 461 - protein, 11 aa, Artificial Sequence
  Description of Artificial Sequence Synthetic peptide
ARLLTRAGAMD

SEQ ID NO: 462 - protein, 11 aa, Artificial Sequence
  Description of Artificial Sequence Synthetic peptide
ARALSRSSGMD

SEQ ID NO: 463 - protein, 11 aa, Artificial Sequence
  Description of Artificial Sequence Synthetic peptide
ARTRFVYYVMD

SEQ ID NO: 464 - protein, 11 aa, Artificial Sequence
  Description of Artificial Sequence Synthetic peptide
ARSLARTSGMD

SEQ ID NO: 465 - protein, 11 aa, Artificial Sequence
  Description of Artificial Sequence Synthetic peptide
ARQITLRGAMD

SEQ ID NO: 466 - protein, 10 aa, Artificial Sequence
  Description of Artificial Sequence Synthetic peptide
ARRRALGAMD

SEQ ID NO: 467 - protein, 10 aa, Artificial Sequence
  Description of Artificial Sequence Synthetic peptide
GFSIGKSGIH

SEQ ID NO: 468 - protein, 10 aa, Artificial Sequence
  Description of Artificial Sequence Synthetic peptide
GFSIRRTDIH

SEQ ID NO: 469 - protein, 10 aa, Artificial Sequence
  Description of Artificial Sequence Synthetic peptide
GFSIRKTDIH

SEQ ID NO: 470 - protein, 10 aa, Artificial Sequence
  Description of Artificial Sequence Synthetic peptide
GFSIGKSGIH

SEQ ID NO: 471 - protein, 10 aa, Artificial Sequence
  Description of Artificial Sequence Synthetic peptide
GFSIGKSGIH

SEQ ID NO: 472 - protein, 14 aa, Artificial Sequence
  Description of Artificial Sequence Synthetic peptide
VAVIYPHDGNTAYA

SEQ ID NO: 473 - protein, 14 aa, Artificial Sequence
  Description of Artificial Sequence Synthetic peptide
VARIYPNSGYTSYA

SEQ ID NO: 474 - protein, 14 aa, Artificial Sequence
  Description of Artificial Sequence Synthetic peptide
VARIYPNSGYTSYA

SEQ ID NO: 475 - protein, 14 aa, Artificial Sequence
  Description of Artificial Sequence Synthetic peptide
VAVIYPHDGNTAYA

SEQ ID NO: 476 - protein, 14 aa, Artificial Sequence
  Description of Artificial Sequence Synthetic peptide
VAVIYPHDGNTAYA

SEQ ID NO: 477 - protein, 12 aa, Artificial Sequence
  Description of Artificial Sequence Synthetic peptide
ARRLALVRMWMD

SEQ ID NO: 478 - protein, 12 aa, Artificial Sequence
  Description of Artificial Sequence Synthetic peptide
ARNVRRRKPTFD

SEQ ID NO: 479 - protein, 12 aa, Artificial Sequence
  Description of Artificial Sequence Synthetic peptide
ARNVRMRKPTLD

SEQ ID NO: 480 - protein, 12 aa, Artificial Sequence
  Description of Artificial Sequence Synthetic peptide
ARRLTLVRMWMD

SEQ ID NO: 481 - protein, 12 aa, Artificial Sequence
  Description of Artificial Sequence Synthetic peptide
ARRLSLVRMWMD

SEQ ID NO: 482 - protein, 9 aa, Artificial Sequence
  Description of Artificial Sequence Synthetic peptide
RLALVRMWM

SEQ ID NO: 483 - protein, 9 aa, Artificial Sequence
  Description of Artificial Sequence Synthetic peptide
NVRRRKPTF

SEQ ID NO: 484 - protein, 9 aa, Artificial Sequence
  Description of Artificial Sequence Synthetic peptide
NVRMRKPTL